(19) United States
(12) Patent Application Publication
(10) Pub. No.: US 2025/0011795 A1
(43) Pub. Date: Jan. 9, 2025

(54) METHODS FOR THE PRODUCTION OF THERAPEUTIC, DIAGNOSTIC, OR RESEARCH ANTIBODIES

(71) Applicant: Wayne State University, Detroit, MI (US)

(72) Inventors: Kang Chen, Detroit, MI (US); Bo Pei, San Diego, CA (US)

(73) Assignee: Wayne State University, Detroit, MI (US)

(21) Appl. No.: 18/766,172

(22) Filed: Jul. 8, 2024

Related U.S. Application Data
(63) Continuation of application No. 17/224,047, filed on Apr. 6, 2021, now Pat. No. 12,065,672.
Application No. 17/224,047 is a continuation of application No. 16/118,090, filed on Aug. 30, 2018, now Pat. No. 11,001,804.
(60) Provisional application No. 62/552,292, filed on Aug. 30, 2017.

(51) Int. Cl. C12N 15/62; A61K 39/00; C07K 14/47; C07K 14/495; C07K 14/54; C07K 14/57; C07K 14/705; C07K 16/06; C12N 5/0781

(52) U.S. Cl. CPC C12N 15/62; A61K 39/4612; A61K 39/4622; A61K 39/464412; A61K 39/464417; A61K 2239/31; A61K 2239/38; C07K 14/4702; C07K 14/495; C07K 14/5406; C07K 14/57; C07K 14/70578; C07K 16/06; C07K 16/065; C12N 5/0635; C07K 2317/565; C07K 2319/21; C07K 2319/41; C07K 2319/43; C12N 2310/20

(57) ABSTRACT

Down-regulating autoimmune regulator (AIRE) function in B cells to produce antibodies is described. The antibodies can be class-switched, high affinity, and neutralizing, and have a high degree of somatic hypermutations, even in the framework region, as compared to antibodies produced in the absence of AIRE downregulation.

### CROSS REFERENCE TO RELATED APPLICATIONS

This is a continuation of co-pending U.S. application Ser. No. 17/224,047, filed Apr. 6, 2021; which claims priority to co-pending U.S. application Ser. No. 16/118,090, filed Aug. 30, 2018, issued as U.S. Pat. No. 11,001,804 on May 11, 2021; which claims priority to and the benefit of the earlier filing date of U.S. Provisional Application No. 62/552,292, filed on Aug. 30, 2017. All prior applications are incorporated by reference in their entirety as if fully set forth herein.

### INCORPORATION BY REFERENCE OF SEQUENCE LISTING

A computer readable file, entitled “MPTDRA Sequence Listing.xml” created on or about Jul. 6, 2024, with a file size of 68,713 bytes, contains the sequence listing for this application and is hereby incorporated by reference in its entirety. The Sequence Listing is being submitted electronically via Patent Center.

### FIELD OF THE DISCLOSURE

The current disclosure provides down-regulating autoimmune regulator (AIRE) function in B cells to produce antibodies that can be high affinity and/or neutralizing. The antibodies produced by the methods disclosed herein can be class-switched and have a high degree of somatic hypermutations as compared to antibodies produced in the absence of AIRE downregulation.

### BACKGROUND OF THE DISCLOSURE

A healthy immune system harbors a properly diversified and selected repertoire of antibodies that is critical for effective immune defense and prevention of autoimmunity. B cells play a major role in this process by producing antigen-specific antibodies against pathogens and imparting immunological memory. For the successful generation of B cell immunity, naive B cell populations with membrane immunoglobulin receptors (B cell receptors, BCRs) recognizing specific antigens are selectively activated in specialized microenvironments called germinal centers (GCs) in secondary lymphatic organs such as the lymph nodes, tonsils, and spleen.

In the weeks following antigenic stimulation in the GC, the specific antibodies that are produced by B cells increase their affinity for the antigen in a gradual and stepwise manner, termed affinity maturation. Affinity maturation involves two interrelated processes: (1) somatic hypermutation (SHM) and (2) clonal selection.

During SHM, mutations are generated in the variable, antigen-binding coding sequences (known as complementarity-determining regions (CDR)) of immunoglobulin genes. The mutation rate is up to 1,000,000 times higher than in cell lines outside of the lymphoid system. The increased mutation rate results in 1-2 mutations per CDR. These mutations alter the binding specificity and binding affinities of the resultant antibodies that are produced.

During clonal selection, B cells that have undergone SHM must compete for growth limiting resources, including the availability of antigen. Follicular dendritic cells (FDCs) of the GCs present antigen to the B cells, and only the B cells expressing BCRs with the higher affinities for the antigen are selected to survive. Over several rounds of selection, the resultant secreted antibodies produced will have effectively increased affinities for the antigen.

Beyond SHM and clonal selection, GC B cells additionally go through class switch recombination (CSR) which is a process that irreversibly rearranges the immunoglobulin (Ig) heavy chain constant region genes. Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgA is similarly subdivided into subclasses including IgA1 and IgA2. Class switch recombination rearranges the immunoglobulin (Ig) heavy chain constant region genes from IgM or IgD to IgG, IgA or IgE. Class switch recombination allows effector function to change while maintaining antigenic specificity. Examples of different effector functions include antibody-dependent cell-mediated cytotoxicity (ADCC); phagocytosis; down regulation of cell surface receptors (e.g. B cell receptors); and B cell activation.

Generation of such long-lived, high affinity class-switched antibodies with neutralization functions protects individuals from re-infection following a first exposure to an antigenic pathogen. This process underlies the success of most vaccines. Antibodies also play large roles in a variety of therapeutic, diagnostic, and research uses.

### SUMMARY OF THE DISCLOSURE

The current disclosure provides systems and methods that improve the ability to generate antibodies against specific antigens. The systems and methods improve the ability to generate antibodies by down-regulating the function of the molecule autoimmune regulator (AIRE) in B cells. Down-regulating AIRE function results in the production of antibodies with increased somatic hypermutation (SHM) and class switch recombination (CSR), thereby facilitating the development of antibodies for clinical and biomedical research applications.

In particular embodiments, the produced antibodies are neutralizing antibodies. In particular embodiments, neutralizing antibodies significantly reduce or block the binding of pathogens and/or their virulence molecules to a host's cellular receptors, such that the pathogens are no longer able to cause cellular damage or enter the host's cells. In particular embodiments, neutralizing antibodies are produced following increased SHM in the FR regions of antibodies. In particular embodiments, the produced antibodies are high affinity antibodies.

### REFERENCE TO SEQUENCE LISTING

The nucleic acid and amino acid sequences listed below are shown using standard letter abbreviations for nucleotide bases, as defined in 37 C.F.R. § 1.822. Only one strand of each nucleic acid sequence is shown, but the complementary strand is understood as included in embodiments where it would be appropriate. The Sequence Listing associated with this application is provided in .XML format in lieu of a paper copy.

In the Sequence Listing:
SEQ ID NO: 1 is the amino acid sequence of AIRE [Homo sapiens]:Accession:CAA08759.1.
SEQ ID NO: 2 is the nucleotide sequence of AIRE [Homo sapiens]: NCBI Reference Sequence:NC_000021.9.
SEQ ID NO: 3 is the amino acid sequence of AIRE [Mus musculus]:Accession:ADZ48462.1.
SEQ ID NO: 4 is the nucleotide sequence of AIRE [Mus musculus]: NCBI Reference Sequence:NC_000076.6.
SEQ ID NO: 5 is the amino acid sequence of CD40 type II isoform [Homo sapiens]:Accession: CAC29424.1.
SEQ ID NO: 6 is the nucleotide sequence of CD40 molecule [Homo sapiens]: NCBI Reference Sequence:NC_000020.11.
SEQ ID NO: 7 is the amino acid sequence of CD40 [Mus musculus]: Accession: AAB08705.1.
SEQ ID NO: 8 is the nucleotide sequence of CD40 antigen [Mus musculus]: NCBI Reference Sequence:NC_000068.7.
SEQ ID NO: 9 is the nucleotide sequence of a representative single guide RNA (sgRNA) for Exon 1 of AIRE.
SEQ ID NO: 10 is the nucleotide sequence of a representative single guide RNA (sgRNA) for Exon 3 of AIRE.
SEQ ID NOs: 11 and 12 are the amino acid sequences of representative protein tags.
SEQ ID NO: 13 AID [Homo sapiens]: Accession: AAM95438.1.

### BRIEF DESCRIPTION OF THE DRAWINGS

The patent or application file contains at least one drawing executed in color. Copies of this patent or patent application publication with color drawings will be provided by the Office upon request and payment of the necessary fee.

Many of the drawings submitted herewith are better understood in color. Applicant considers the color versions of the drawings as part of the original submission and reserve the right to present color images of the drawings in later proceedings.

FIGS. 1A-1H. GC B cells express AIRE in a CD40-dependent manner. FIG. 1A, Immunofluorescence analysis of the tonsillar tissue of a healthy donor for IgD, CD19, AIRE and DAPI-stained DNA, and the thymic tissue of a healthy donor for EpCAM, AIRE and DNA. The dotted line marks the boundary between tonsil follicular mantle zone and the follicle. Bars: 20 μm. The results represent 5 healthy tonsil donors and 2 healthy thymus donors. FIG. 1B, Flow cytometric analysis of AIRE expression in tonsillar CD19⁺IgD⁺CD38⁻ naive B cells, CD19³⁰ IgD⁺CD38⁺ FGC B cells, CD19⁺IgD⁻CD38⁺ GC B cells and CD19⁺IgD⁻CD38⁻ memory B cells. The data represent those from 5 donors. FIG. 1C, Immunofluorescence analysis of the splenic tissue of a B6 mouse immunised with 3 doses of sheep red blood cells (SRBCs) for IgD, AIRE, CD19 and DNA, and the thymic tissue of an immunised B6 mouse for UEA-1, AIRE and DNA. Bar: 20 μm. The results represent the splenic tissues of 5 mice and the thymic tissue of 3 mice. FIG. 1D, FIG. 1E, Flow cytometric and statistical analyses of AIRE (GFP) expression in splenic and ILN viable CD19⁺B220⁺FAS⁺GL7⁺ GC B cells, CD19⁺B220+FAS⁻GL7⁻ non-GC B cells and CD19^{Io}B220^{Io}CD138⁺ plasma cells (PCs) of B6 mice (shaded histograms, n=4) or B6.Aire^{Adig} mice (histograms, n=4) after immunization with 5 doses of NP₃₂-KLH. The results represent or compare 4 B6 and 4 B6.Aire^{Adig} mice. FIG. 1F, Flow cytometric analysis of CD83 and CXCR4 expression on total and GFP⁺ splenic GC B cells of an immunised B6.Aire^{Adig} mouse, showing the distribution of GFP⁺ GC B cells in CXCR4^{hi}CD83⁻ DZ and CXCR4^{Io}CD83⁺ LZ B cells. The result represents 4 B6.Aire^{Adig} mice. FIG. 1G, Immunofluorescence analysis of the tonsillar tissue of a HIGM3 patient for IgD, AIRE, CD19 and DNA. The dotted line marks the boundary between follicular mantle zone and the follicle. Bars: 20 μm. FIG. 1H, Flow cytometric analysis of AIRE (GFP) expression in splenic B cells of a B6 or B6.Aire^{Adig} mouse treated for 3 d with medium or CD40L with or without IL-4 in the absence (vehicle) or presence of CAPE. The data represent the results from 3 B6 and 3 B6.Aire^{Adig} mice. ***P<0.01, ***P<0.001, by 2-tailed t-test.

FIGS. 2A-2H. GC B cells in secondary lymphoid tissues express AIRE. FIGS. 2A-2D, Immunofluorescence analysis of tonsillar (2A, 2B) and splenic (2C, 2D) tissues of healthy donors for IgD, AIRE, CD19 and DNA, showing the presence of AIRE in the nuclei of follicular GC B cells. Follicular IgD⁺ plasmablasts (arrow heads) did not contain AIRE. The areas 1 and 2 outlined in FIG. 2A and FIG. 2C are shown in with a higher magnification of FIG. 2B and FIG. 2D, respectively. Dotted lines mark the boundary between follicular mantle zone and the follicle. Bars: 40 μm (FIG. 2A) and 15 μm (FIG. 2B). FIG. 2E, Flow cytometric gating strategy for identifying human peripheral blood naive (IgD⁺CD27⁻), MZ (IgD⁺CD27⁺), switched memory (IgD⁻CD27+), double-negative (IgD⁻CD27⁻) B cells, and transitional (CD24^{hi}CD38^{hi}), mature (CD24^{int}CD38^{int}), memory (CD24^{hi}CD38⁻) B cells and plasma cells (CD24⁻CD38^{hi}). 2F, AIRE expression in human peripheral blood B cell subsets, as determined by flow cytometry. The result is representative of 8 healthy donors. FIG. 2G, Flow cytometric gating strategy for identifying mouse splenic non-GC (CD19⁺B220⁺GL7⁻FAS⁻), GC (CD19⁺B220⁺GL7⁺FAS⁺) B cells and plasma cells (CD19^{Io}B220^{Io}CD138⁺). FIG. 2H, AIRE expression in mouse peripheral blood, splenic, MLN, PP and thymic B cells of B6.Aire^{Adig} mice. The data are representative of 12 B6.Aire^{Adig} and 6 B6 mice that were age- and sex-matched and housed in the same SPF room.

FIGS. 3A-3D. AIRE expression in GC B cells requires CD40 signalling. FIGS. 3A-3D, Immunofluorescence analysis of tonsillar tissues of a healthy donor and a HIGM3 patient for IgD, AIRE, CD19 and DNA, showing the lack of AIRE expression in GC B cells in the HIGM3 patient. The HIGM3 tonsil harbors giant follicles with defective follicular Ig class switch recombination (CSR) and hence showing follicular IgD staining without follicular IgD⁺ plasmablasts, although extrafollicular and subepithelial IgD⁺ plasmablasts are present and generated via T cell-independent mechanisms. Chen et al., Nature Immunology 10, 889-898 (2009). The areas outlined in FIG. 3A and FIG. 3C are shown with a higher magnification in FIG. 3B and FIG. 3D, respectively. The dotted lines outline the follicles. Bars: 100 μm (FIG. 3A, 3C) or 25 μm (FIG. 3B, 3D).

FIGS. 4A-4F. AIRE expression in B cells is induced by CD40 ligation in vitro. FIG. 4A, FIG. 4B, qRT-PCR and Western Blot analyses of AIRE transcript and protein levels, the protein levels of total and Ser536-phosphorylated NF-_{K}B p65, and total and Thr202/Tyr204-phosphorylated Erk½ in human peripheral blood IgD⁺ B cells treated with medium or CD40L, or CD40L and IL-4, in the presence of vehicle or CAPE for 3 d. FIG. 4C, FIG. 4D, qRT-PCR and Western Blot analyses of AIRE transcript and AIRE protein levels in human 2E2 B cells treated with medium (Control) or CD40L and IL-21 for 3 d. FIG. 4E, FIG. 4F, qRT-PCR and Western Blot analyses of Aire transcript and AIRE protein levels in mouse CH12 cells treated with anti-CD40, TGF-β1 and ng/ml IL-4 for 3 d. *P<0.05, **P<0.01, ***P<0.001, by 2-tailed t-test. The data represent 3-4 experiments.

FIGS. 5A-5I. Aire^{+/+} and Aire^{-/-} B cells had a similar phenotype before transfer and entered GC reaction equally in immunised μMT recipients in vivo, and exhibited similar proliferation and apoptosis during ex vivo stimulation. FIG. 5A, Purity of Aire^{+/+} and Aire^{-/-} littermate donor B cells before adoptive transfer. FIG. 5B, Cell surface expression of the differentiation and activation markers CD21, CD23, CD38, CD40, CD62L, CD80, CD86, CD93, I-A^{b}, BAFF-R and immunoglobulin IgM and IgD on purified Aire^{+/+} and Aire^{-/-} littermate donor B cells before adoptive transfer, as determined by flow cytometry. FIG. 5C, Percentage of GL7⁺FAS⁺ GC B cells in the spleens of μMT recipients of either Aire^{+/+} or Aire^{-/-} B cells that were immunised i.p. with 5 doses of NP₃₂-KLH. Flow cytometry was performed 4 d after the last immunization. FIG. 5D, Cell surface expression of the co-stimulatory or co-inhibitory molecules CD80, CD86, PD-L1, PD-L2 and ICOSL on GL7⁺FAS⁺ GC B cells in the spleens of μMT recipients after immunizations. Shaded histograms indicate the staining using isotype-matched control antibodies. FIG. 5E, 5F, Percentage of splenic PD-1⁺CXCR5⁺ T_{FH} cells and PD-1⁺CXCR5⁺Foxp3⁺CD25⁺ T_{FR} cells in the spleens of immunised μMT recipients. The results shown represent 4 experiments, each including B cells from 3-5 age- and sex-matched littermate donor mice and 6-8 age- and sex-matched littermate μMT recipient mice. FIG. 5G, CFSE dilution in purified B cells from age- and sex-matched littermate donor Aire^{+/+} and Aire^{-/-} mice treated with medium (Control) or CD40L and IL-4 for 5 or 7 d. Non-viable cells were excluded from the analysis. FIG. 5H, Six-hour EdU incorporation by Aire^{+/+} or Aire^{-/-} cells stimulated for 5 d with CD40L and IL-4. FIG. 5I, Apoptosis of Aire^{+/+} or Aire^{-/-} B cells treated with medium (Control) or CD40L and IL-4 for 3 or 7 d, as determined by Annexin V and 7-AAD staining by flow cytometry. The results shown are representative of 3 experiments, each including cells from 2-3 age- and sex-matched littermate Aire^{+/+} and Aire^{-/-} mice.

FIGS. 6A-6H. AIRE in B cells inhibits Ig diversification. FIG. 6A, Flow cytometric analysis of surface IgD and IgM on NP₃₆-binding B cells in μMT recipients of Aire^{+/+} or Aire^{-/-} B cells immunised with 5 doses of NP₃₂-KLH. The result represents 3 age- and sex-matched μMT recipients each of B cells from 3-5 age- and sex-matched littermate donor Aire^{+/+} or Aire^{-/-} mice. FIG. 6B, The ratios of the titers of circulating NP₄-binding to NP₂₉-binding IgM, IgG1, IgG2b and IgG3 in immunized μMT recipient mice of Aire^{+/+} or Aire^{-/-} B cells. The results represent 4 experiments, each including B cells from 3-5 age- and sex-matched littermate donor mice and 6-8 age- and sex-matched littermate μMT recipient mice. FIG. 6C, Flow cytometric analysis of surface IgM, IgG1 and IgA by Aire^{+/+} or Aire^{-/-} mouse splenic B cells stimulated ex vivo for 4 d. The results represent 3 experiments. FIG. 6D, ELISA of IgG1 and IgA in supernatants of Aire^{+/+} or Aire^{-/-} mouse splenic B cells stimulated ex vivo for 5 d with anti-CD40 and IL-4 (for IgG1) or anti-CD40, TGF-β and IL-4 (for IgA). The results represent 3 experiments. FIG. 6E, 6F, Flow cytometric and statistical analyses of IgA CSR in WT and Aire^{-/-} CH12 cells treated with medium (Control) or stimulated with anti-CD40, TGF-β and IL-4 for 3 d. Relative CSR (FIG. 6E) was determined as the ratio of the percentages of IgA⁺IgM⁻ cells in stimulated samples to control samples followed by normalization of such ratios by setting the values of WT CH12 cells to 1. The results represent or compare 3 experiments involving WT, clones 43, 53 and 69, and 11 additional experiments involving WT and clone 69. FIG. 6G, qRT-PCR analysis of the Iα-Cμ circle transcript levels in WT and Aire^{-/-} CH12 cells treated with medium (Control) or stimulated with anti-CD40, TGF-β and IL-4 for 3 d. The results compare 3 experiments. FIG. 6H, Flow cytometric analysis of IgA CSR in Aire^{-/-} CH12 cells (clone 69) transfected with a construct expressing either NLS-deficient AIRE-GFP (AIRE^{ΔNLS}-GFP) or WT AIRE-GFP (AIRE^{WT}-GFP) and treated with medium (Control) or stimulated with anti-CD40, TGF-β and IL-4 for 3 d. The results represent 2 experiments. *P<0.05, **P<0.01, ***P<0.001, by 2-tailed t-test (FIGS. 6B, 6D, 6F) or 1-tailed t-test (FIG. 6G).

FIGS. 7A-7F. Validation of Aire^{-/-} CH12 cell clones. FIGS. 7A-7C, DNA sequencing (right panels) of the Aire gene (SEQ ID NO: 4) showing CRISPR-introduced mutations causing frame shift in both alleles. The deleted nucleotides are shaded in dark gray (top panels) with the deletion site indicated by dark gray arrows (bottom panels) in the sequencing results. The axons of Aire are shaded in light gray, with the amino acid translation shown above the nucleotide sequence and the stop codon introduced shaded in gray and marked with an asterisk. FIG. 7D, Verification of Aire mutations in CH12 clones by PCR using primers that only anneal to the WT sequence, giving no amplification in clones 43, 47 and 53. Clone 47 has a 3-bp deletion in both Aire alleles causing a single amino acid deletion, and hence was not used in experiments. FIG. 7E, Verification of Aire mutations in both alleles of CH12 clone 69 by PCR showing no amplification using primer pair #2 which anneals to the WT but not the mutated sequence. Primer pair #1 amplifies a sequence immediately downstream of the mutation site, and primer pair #3 is specific for the single-stranded repair template used in CRISPR. FIG. 7F, Western Blot analysis of AIRE protein expression in WT and Aire^{-/-} CH12 cells.

FIGS. 8A-8F. AIRE deficiency does not affect the expression of AID or germline transcripts in CH12 cells. FIG. 8A, qRT-PCR analysis of the ly1-Cμcircle transcript level in Aire^{+/+} CH12 cells and Aire^{-/-} CH12 cell clones 43, 53 and 69 that were either unstimulated or stimulated with anti-CD40, TGF-β1 and IL-4 for 3 days. The result was normalised using the respective Actb transcript level, and expressed as fold of induction relative to unstimulated Aire^{+/+} CH12 cells. The data are representative of three experiments. FIGS. 8B, 8C, Western Blot analysis of AID in WT and Aire^{-/-} CH12 cells that were either unstimulated or stimulated with anti-CD40, TGF-β1 and IL-4 for 3 d. Lamin B1 and GAPDH were used as the control for nuclear and cytoplasmic proteins, respectively. The data are representative of 2 experiments. FIG. 8D, qRT-PCR analysis of Aicda and the Iμ-Cμand Iα-Cα germline transcript levels in Aire^{+/+} CH12 cells and Aire^{-/-} CH12 cell clones 43, 53 and 69 that were either unstimulated or stimulated with anti-CD40, TGF-β1 and IL-4 for 3 d. FIG. 8E, Flow cytometric analysis of apoptosis by Annexin V and 7-AAD staining of WT and Aire^{-/-} CH12 cells treated with medium (Control) or stimulated with anti-CD40, TGF-β1 and IL-4 for 3 d. FIG. 8F, Percentages of late apoptotic (Annexin V⁺7-AAD⁺) and early apoptotic (Annexin V⁺7-AAD⁻) in WT and Aire^{-/-} CH12 cells treated with medium (Control) or stimulated with anti-CD40, TGF-β1 and IL-4 for 3 d. *P<0.05, by 2-tailed t-test. The data represent 4 experiments.

FIGS. 9A-9J. AIRE interacts with AID in GC B cells and inhibits AID activity by reducing AID targeting to Ig S region and stalled Pol II. FIG. 9A, Imaging flow cytometric analysis of AIRE and AID in tonsillar IgD⁻CD38⁺ GC B cells of a healthy donor. Bars: 7 μm. The results represent 3 donors. FIG. 9B, FIG. 9C, Co-IP of AIRE and AID in tonsillar CD19⁺ total, IgD⁺ naive and FGC and CD19⁺IgD⁻GC and memory B cells of a healthy donor, and in splenic CD19⁺ B cells of a B6 mouse after 3 doses of immunization with SRBCs. The results are representative of tonsils of 4 donors and spleens of 3 mice. FIG. 9D, The domain structures of recombinant WT and mutant human AIRE and AID molecules. Dotted lines indicated the deleted regions in the mutant proteins. FIG. 9E, Co-IP of WT AID and WT or mutant AIRE in HKB-11 cells 24 h after transfection of plasmid(s) encoding WT AID and WT or mutant AIRE proteins. FIG. 9F, The domain structures of recombinant WT and mutant human AID molecules. FIG. 9G, Co-IP of WT AIRE and WT or mutant AID in HKB-11 cells 24 h after transfection of plasmid(s) encoding WT AIRE and WT or mutant AID proteins. The results in FIG. 9E and FIG. 9G are representative of 3 experiments. FIG. 9H, A dot blot assay for the genomic uracil content in WT and Aire^{-/-} CH12 cells after 48 or 72 h of treatment without or with anti-CD40, TGF-β and IL-4. The results represent 3 experiments. FIG. 9I, ChIP-qPCR analysis for the interaction of AID with Sμ, Iμ and Sy1 regions in WT and Aire^{-/-} CH12 cells after 72 h of treatment without or with anti-CD40, TGF-β and IL-4. The results represent 3 experiments. FIG. 9J, Co-IP of AID with pSer5-Pol II, total Pol II, Spt5 and AIRE in WT and Aire^{-/-} CH12 cells after 72 h of treatment without or with anti-CD40, TGF-β and IL-4. The results represent 3 experiments. *P<0.05, **P<0.01, ***P<0.001, by 2-tailed t-test.

FIGS. 10A-10F. AIRE and AID co-localize in the nuclei of GC B cells. FIG. 10A, The gating strategy to identify tonsillar naive (IgD⁺CD38⁻), founder GC (FGC) (IgD⁺CD38⁺), GC (IgD⁻CD38⁺) and switched memory (IgD⁻CD38⁻) B cells and switched plasma cells (PCs) (IgD⁻CD38^{hi}) on the imaging flow cytometer. The plot displays MACS-purified CD19⁺ tonsillar B cells. FIGS. 10B-10F, Imaging flow cytometry of AIRE and AID in tonsillar GC, naive, FGC, switched memory B cells and switched PCs of a healthy donor. DNA was counterstained with DAPI. Samples stained with isotype-matched control antibodies were used to define the fluorescence baseline for AIRE and AID. Four representative cells in each population stained with AIRE and AID or with isotype control antibodies were shown. Bars: 7 μg.

FIGS. 11A-11E. AIRE interacts with AID but not Bcl10 in B cells, and inhibits AID activity. FIG. 11A, Co-IP of AIRE and AID in splenic B cells of immunised WT or Aicda^{-/-} mice. The data represent 2 experiments. FIG. 11B, Western Blot analysis of Bcl10 in cytoplasmic and nuclear extracts of Ramos B cells unstimulated or stimulated with TNF for 24 h. FIG. 11C, Co-IP of AID and AIRE or Bcl10 in Ramos B cells unstimulated or stimulated with TNF for 24 h. The data in FIG. 11B and FIG. 11C represent 3 experiments. FIG. 11D, The principle of the uracil dot blot assay for the quantitation of genomic uracil. In stimulated Aire^{+/+} or Aire^{-/-} B cells, AID deaminates C to U in Ig V and S regions. U is excised by either endogenous Uracil N-glycosylase (UNG) or exogenously added Escherichia coli Uracil DNA glycosylase (UDG) during the assay to generate abasic sites (asterisk), which exist in an equilibrium between the closed and open ring forms. The active aldehyde in the open ring form reacts with the biotinylated aldehyde-reactive probe (Biotin-ARP), allowing biotinylation of the abasic site. Quantitation of biotinylated abasic sites with fluorochrome- or HRP-conjugated streptavidin after DNA dot blot by imaging or ELISA gives genomic U content. An increase in genomic U content in Aire^{-/-} B cells indicates increased activity of AID in the absence of AIRE. FIG. 11E, A representative standard calibration curve of the fluorescence intensity vs. uracil number of the assay.

FIGS. 12A-12C. Primers for cloning human AIRE and AID constructs.

FIGS. 13A, 13B. Aire^{-/-}/ donor B cells class-switched to IgG or IgE have increased IgH CDR2 SHMs than their Aire^{+/+} counterparts after repeated immunizations. FIG. 13A, The sorting and sequencing strategies for Aire^{+/+} and Aire^{-/-} donor B cells in μMT recipients after immunizations with 5 doses of NP₃₂-KLH. NP-specific B cells were sorted based on NP₃₆ binding. FIG. 13B, The SHM landscape across IgHV, including FR2, CDR2, FR3, CDR3 and FR4, of NP₃₆-binding IgM⁻IgD⁻ or IgM⁺IgD⁺ Aire^{+/+} and Aire^{-/-} donor B cells in μMT recipients after immunizations with NP₃₂-KLH. The result represents 3 μMT recipients of Aire^{+/+} donor B cells and 3 μMT recipients of Aire^{-/-} donor B cells.

FIGS. 14A-14H. AIRE deficiency in B cells promotes humoral autoimmunity and compromises cutaneous anti-Candida defense. FIG. 14A, Frequencies of C-to-T transitions in SHMs in IgHV of NP-specific IgG⁺, IgA⁺ or IgE⁺ splenic B cells from μMT recipient mice of Aire^{+/+} or Aire^{-/-} B cells after 5 doses of immunizations with NP₃₂-KLH. FIG. 14B, qRT-PCR analysis of the fold induction of Iμ-Cy1 and Iμ-Cy3 post-switch transcript levels in peripheral blood IgD⁺CD27⁻ naïve B cells from healthy subjects (n=5) or APS-1 patients (n=5) stimulated for 3 d with CD40L and IL-4 or IFN-y over the respective unstimulated control B cells. FIGS. 14C, 14D, GMS stain of cutaneous C. albicans and skin fungal burden (CFU per mg of tissue) in μMT recipient mice of Aire^{+/+} or Aire^{-/-} donor B cells 4 d after infection. Bars: 1 mm (FIG. 14C, upper panels) or 100 μm (FIG. 14C, lower panels). FIG. 14E, Levels of autoantibodies binding to IL-17A, IL-17F and IL-22 in the sera of μMT recipient mice of Aire^{+/+} or Aire^{-/-} donor B cells 4 d after infection. FIG. 14F, Flow cytometric analysis of IL-17A and IL-22 expression in cutaneous CD4⁺ T cells of μMT recipient mice of Aire^{+/+} or Aire^{-/-} donor B cells 4 d after infection and after ex vivo re-stimulation. FIG. 14G, Immunofluorescence analysis of Ly-6G (red) and DNA (blue) in cutaneous tissues surrounding the C. albicans infection site in μMT recipient mice of Aire^{+/+} or Aire^{-/-} donor B cells 4 d after infection. The results in FIGS. 14C-14G represent 1 of 2 experiments, with 4 mice per group in each experiment. FIG. 14H, A simplified schematic of AIRE-mediated GC checkpoint of antibody diversification in B cells. At the T-B cell border of secondary lymphoid organs, B cells present antigens to and receive co-stimulation from DC-activated T cells, which also induce AIRE expression in B cells via CD40. The activated B cells enter the GC DZ and undergo SHM, proliferation and subsequent affinity selection by interacting with antigens on the surface of follicular dendritic cells (FDCs) in LZ. Low-affinity B cells will undergo apoptosis, whereas high-affinity B cells receive help from T follicular helper (TFH) cells to undergo CSR, and subsequently either re-enter the SHM-proliferation cycle in the DZ or exit the GC as plasma cells or memory B cells. AIRE in B cells limits autoantibody generation by restraining excessive AID activity in the GC. *P<0.05, **P<0.01, by 1-tailed t-test (FIGS. 14A, 14B left panel, FIGS. 14D, 14E) or 1-tailed Mann-Whitney U test (b right panel).

FIGS. 15A-15C. AIRE deficiency in B cells impairs T_{H}17 immunity against cutaneous C. albicans infection. FIG. 15A, Flow cytometric gating strategy for identifying mouse skin viable CD45⁺TCRyδ⁺TCRβ⁻yδ T cells, CD45⁺TCRyδ⁻TCRβ⁺CD3⁺CD4⁺CD4⁺ T cells and CD45⁺TCRyδ⁻TCRβ⁺CD3⁺CD4⁻CD8⁺ T cells. FIG. 15B, IHC of CD4⁺ T cells in cutaneous tissues surrounding the infection site in μMT recipient mice of Aire⁺⁺ or Aire^{-/-} donor B cells 4 d after infection. Bars: 100 μm. FIG. 15C, Flow cytometric analysis of IL-17A and IL-22 expression in cutaneous CD8⁺ and yδ T cells of μMT recipient mice of Aire^{+/+} or Aire^{-/-} donor B cells 4 d after infection. Data represent 2 experiments.

FIGS. 16A, 16B. qRT-PCR primers used in Example 1.

FIGS. 17A, 17B, 17D, 17E. Antibodies used in Example 1.

### DETAILED DESCRIPTION

A healthy immune system harbors a properly diversified and selected repertoire of antibodies that is critical for effective immune defense and prevention of autoimmunity. Mature B cells undergo antigen-driven antibody diversification via somatic hypermutation (SHM) and class-switch recombination (CSR) mediated by the enzyme activation-induced cytidine deaminase (AID) in germinal centers (GCs) of secondary lymphoid organs. Muramatsu et al., Cell 102, 553-563, (2000); Revy et al., Cell 102, 565-575, (2000).

Uncontrolled AID function can precipitate autoimmunity and cancer. Vinuesa et al., Nature reviews. Immunology 9, 845-857, (2009); Casellas et al. Nature reviews. Immunology 16, 164-176, (2016). Mutations in the autoimmune regulator (AIRE) gene, which normally promotes central and peripheral T cell tolerance (Anderson et al., Science 298, 1395-1401, (2002); Gardner et al., Science 321, 843-847, (2008); Malchow et al., Science 339, 1219-1224, (2013)), cause autoimmune polyglandular syndrome type 1 (APS-1) (Nagamine et al.,Nature genetics 17, 393-398, (1997); Finnish-German, Nature genetics 17, 399-403, (1997)) associated with aberrant production of autoantibodies by B cells, organ-specific autoimmunity and increased susceptibility to mucocutaneous Candida albicans infection. Anderson et al., Science 298, 1395-1401, (2002).

The current disclosure provides that AIRE in GC B cells inhibits immunoglobulin affinity maturation (e.g., SHM) and CSR. When AIRE function is down-regulated, antigen-specific B cells develop into antibody-secreting plasma cells that produce antibodies with increased affinity and/or neutralization function. The down-regulation of AIRE improves antibody production not only in cultured B cells, but also in mouse models with AIRE deficiency. Moreover, in AIRE-deficient mice, there is an increased population of helper T cells (Tfh). Tfh cells are a type of T cell that specializes in promoting GC B cells to produce high affinity class-switched antibodies and evoking a more rapid memory B cell response to previously encountered antigens.

The systems and methods of the disclosure can be used for the generation of antibodies in both the primary immune response and the recall immune response in vivo as well as in B cell cultures in vitro. Uses of generated antibodies include therapeutic uses (e.g., antibody-based therapeutics for cancer, autoimmune, and inflammatory diseases) and diagnostic and/or research uses (e.g., flow cytometry, imaging, immunohistochemistry, western blot).

Particular embodiments include increasing SHM and CSR during antibody production by selecting a first population of B cells with down-regulated AIRE function; and contacting the selected B cell population with an antigen; thereby increasing SHM and CSR during antibody production. In particular embodiments, the increase can be in relation to antibodies produced by selecting a second population of B cells with normal AIRE function; and contacting the second selected B cell population with the same antigen under comparable conditions.

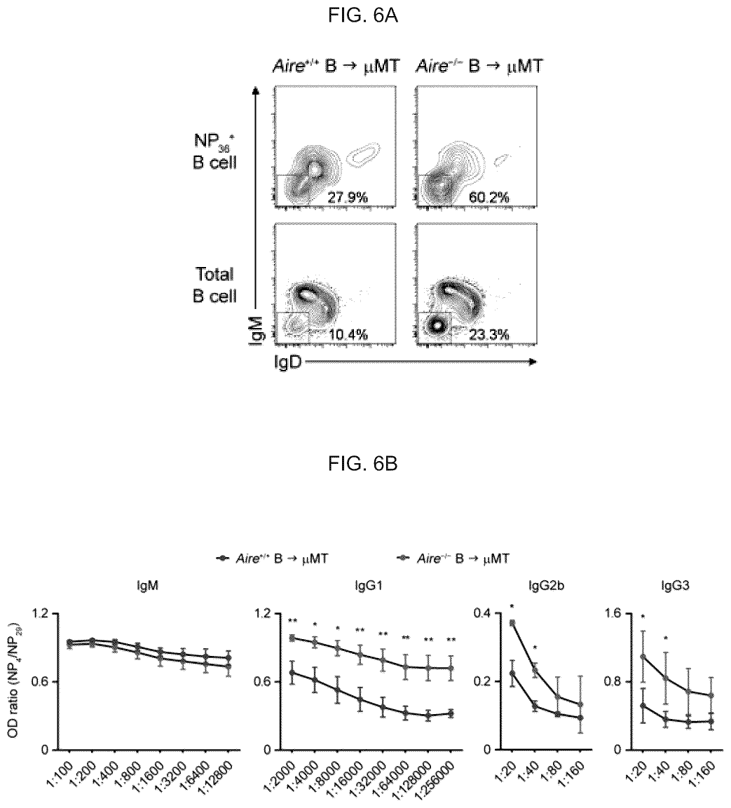

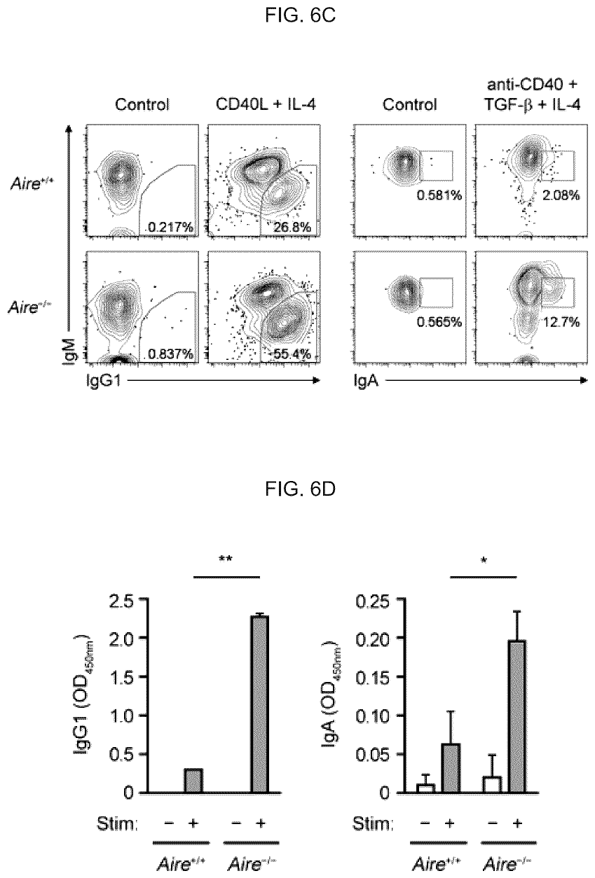

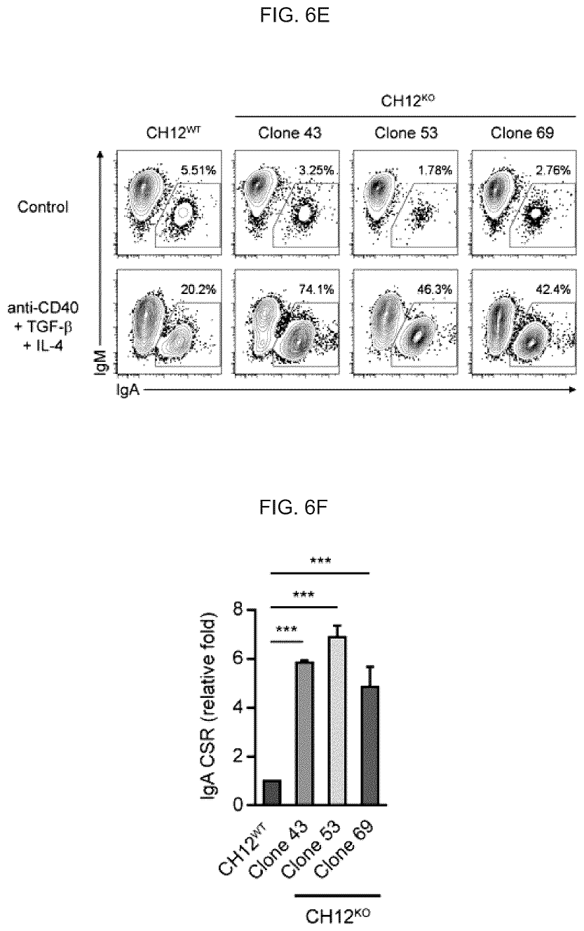

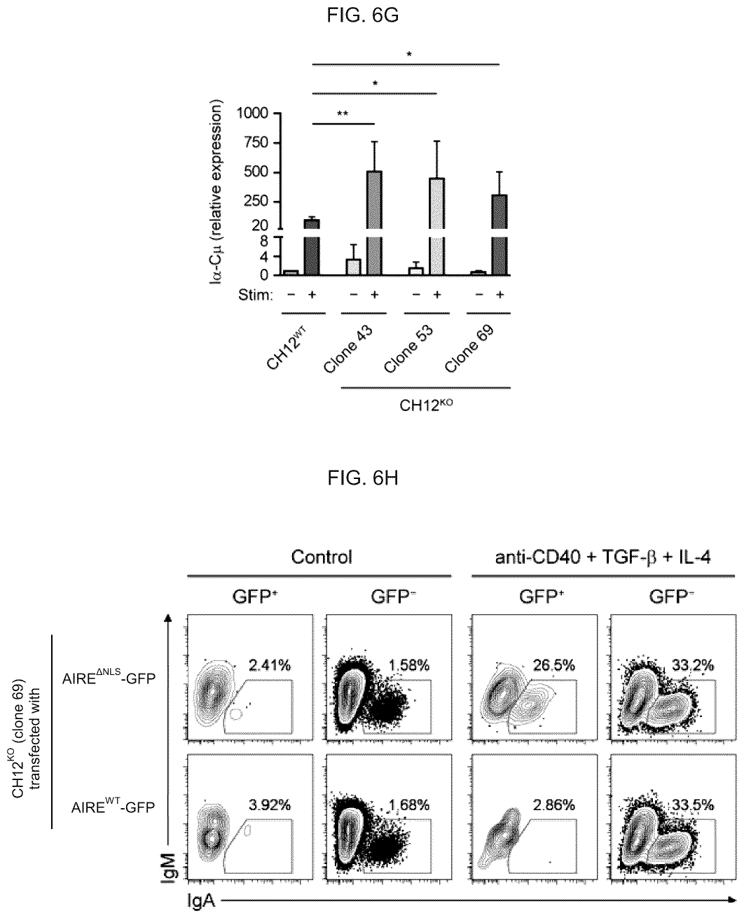

In particular embodiments, increased SHM can be confirmed by the methods used to generate the data presented in FIG. 6B. Briefly, antigens of a lower oligomeric number (in this case NP₄) and those of a higher oligomeric number (in this case NP₂₉) are coated onto the wells of microtiter plates. Samples containing the antibodies whose affinity is to be determined are applied to the coated microtiter plates. Antibodies of high affinity would be able to bind to the antigens with a lower oligomeric number, and those of both high and low affinity would be able to bind to the antigens with a higher oligomeric number. The ratios of binding to the lower oligomeric antigens to the higher oligomeric antigens would be an indication of the proportion of high-affinity antibodies in a given sample. In particular embodiments, the increase can be a statistically-significant increase.

In particular embodiments, increased CSR can be confirmed by the methods used to generate the data presented in FIGS. 6C, 6D, 6E, 6F and 6G. Briefly, the expression of cell surface immunoglobulin is measured by flow cytometry. A reduction in IgM, the antibody expressed before CSR, and an increase in various subclasses of IgG, IgA or IgE expression on the cell surface indicates CSR. In addition, the secretion of the various classes of IgG, IgA or IgE into the supernatant would also increase after the B cells are class switched. In particular embodiments, the changes can be statistically-significant changes.

In particular embodiments, “under comparable conditions” refers to experimental conditions under which one of ordinary skill the art would expect similar results (i.e., lack of a statistically significant difference between groups), but for an experimental variable.

Particular embodiments include methods of producing neutralizing antibodies against a pathogen by selecting a population of B cells with down-regulated AIRE function; and contacting the selected B cell population with an antigen of the pathogen; thereby producing neutralizing antibodies against the pathogen.

Function as a neutralizing antibody can be shown through the ability to block or reduce cellular function as measured by flow cytometry. In particular embodiments percent neutralization can refer to a percent decrease in infectivity in the presence of an antibody, as compared to pathogen infectivity in the absence of the antibody. For example, if half as many cells in a sample become infected in the presence of an antibody, as compared to in the absence of the antibody, this can be calculated as 50% neutralization. In particular embodiments neutralizing antibodies result in at least 40% neutralization, at least 50% neutralization, at least 60% neutralization, at least 70% neutralization, at least 80% neutralization, or at least 90% neutralization. In particular embodiments, antibodies produced according to the disclosure can block infection by a pathogen (i.e., 100% neutralization).

Particular embodiments include methods of producing antibodies with high affinity for an antigen by selecting a population of B cells with down-regulated AIRE function; and contacting the selected B cell population with the antigen; thereby producing antibodies with high affinity for the antigen.

In particular embodiments, high affinity can be confirmed by an increased ratio of binding to NP₄ to NP₂₉ of the antibodies made by Aire^{-/-} B cells than those made by Aire^{+/+} B cells under comparable conditions. In particular embodiments, the increased ratio can be statistically-significant.

Particular embodiments include kits for producing antibodies with increased SHM and CSR including: a B cell population with down-regulated AIRE function; and an antigen.

Particular embodiments include kits for producing antibodies with increased SHM and CSR including: a B cell population; gene editing agents to down-regulate AIRE function and/or CD40 function in the B cell population; and an antigen.

Aspects of the current disclosure are now described in more detail.

Embodiments disclosed herein include utilizing B cells with down-regulated AIRE activity. In particular embodiments, B cells may naturally have down-regulated AIRE function, as compared to a reference level. Reference levels can include “normal” or “control” levels or values, defined according to, e.g., discrimination limits or defining thresholds, in order to define down-regulated AIRE function. The reference level can include AIRE function levels typically found in natural B cells not associated with a condition that includes down-regulated AIRE function. Other terms for “reference levels” include “index,” “baseline,” “standard,” “healthy,” etc. In particular embodiments, “reference level” can refer to a standardized control value for normal AIRE function which represents levels not associated with any pathological disease or condition.

In particular embodiments, B cells may have experimentally down-regulated AIRE function. In these embodiments, normal AIRE function refers to AIRE function observed in the absence of an experimental procedure to down-regulate AIRE function. Down-regulated AIRE function refers to AIRE function following an experimental procedure to down-regulate AIRE function. The different levels can be compared to confirm down-regulated AIRE function, as is understood by one of ordinary skill in the art.

Any method to down-regulate AIRE function can be used. Particular embodiments can utilize gene-editing agents. As used herein, gene editing agents modify or affect a B cell's endogenous genome. In particular embodiments, the modification includes removal or disruption of an endogenous gene such that the endogenous gene's encoded protein is no longer expressed, expressed to a reduced degree, expressed as an incomplete protein, an unstable protein, an incorrectly folded protein and/or a nonfunctional protein. For example, as disclosed herein, AIRE mutants missing one or more of the N-terminal caspase activation and recruitment domain (CARD) and/or nuclear localization signal (NLS) lose the ability to interact with AID. Thus, these forms of AIRE are down-regulated. The current disclosure also provides that AIRE expression in B cells is dependent on CD40 signaling. Accordingly, AIRE function can be down-regulated by interfering with CD40 expression and/or signaling.

Particular embodiments utilize CRISPR-Cas to down-regulate AIRE. CRISPR-Cas systems include CRISPR repeats and a set of CRISPR-associated genes (Cas).

The CRISPR repeats (clustered regularly interspaced short palindromic repeats) include a cluster of short direct repeats separated by spacers of short variable sequences of similar size as the repeats. The repeats range in size from 24 to 48 base pairs and have some dyad symmetry which implies the formation of a secondary structure, such as a hairpin, although the repeats are not truly palindromic. The spacers, separating the repeats, match exactly the sequences from prokaryotic viruses, plasmids, and transposons. The Cas genes encode nucleases, helicases, RNA-binding proteins, and a polymerase that unwind and cut DNA. Cas1, Cas2, and Cas9 are examples of Cas genes.

The source of CRISPR spacers indicate that CRISPR-Cas systems play a role in adaptive immunity in bacteria. There are at least three types of CRISPR-Cas immune system reactions, and Cas1 and Cas2 genes are involved in spacer acquisition in all three. Spacer acquisition, involving the capture and insertion of invading viral DNA into a CRISPR locus occurs in the first stage of adaptive immunity. More particularly, spacer acquisition begins with Cas1 and Cas2 recognizing invading DNA and cleaving a protospacer, which is ligated to the direct repeat adjacent to a leader sequence. Subsequently, single strand extension repairs take place and the direct repeat is duplicated.

The next stage of CRISPR-related adaptive immunity involves CRISPR RNA (crRNA) biogenesis, which occurs differently in each type of CRISPR-Cas system. In general, during this stage, the CRISPR transcript is cleaved by Cas genes to produce crRNAs. In the type I system, Cas6e/Cas6f cleaves the transcript. The type II system employs a transactivating (tracr) RNA to form a dsRNA, which is cleaved by Cas9 and RNase III. The type Ill system uses a Cas6 homolog for cleavage.

In the final stage of CRISPR-related adaptive immunity, processed crRNAs associate with Cas proteins to form interference complexes. In type I and type II systems, the Cas proteins interact with protospacer adjacent motifs (PAMs), which are short 3-5 bp DNA sequences, for degradation of invading DNA, while the type III systems do not require interaction with a PAM for degradation. In the type III-B system, the crRNA basepairs with the mRNA, instead of the targeted DNA, for degradation.

CRISPR-Cas systems thus function as an RNAi-like immune system in prokaryotes. The CRISPR-Cas technology has been exploited to inactivate genes in human cell lines and cells. As an example, the CRISPR-Cas9 system, which is based on the type II system, has been used as an agent for genome editing.

The type II system requires three components: Cas9, crRNA, and tracrRNA. The system can be simplified by combining tracrRNA and crRNA into a single synthetic single guide RNA (sgRNA).

At least three different Cas9 nucleases have been developed for genome editing. The first is the wild type Cas9 which introduces double strand breaks (DSBs) at a specific DNA site, resulting in the activation of DSB repair machinery. DSBs can be repaired by the non-homologous end-joining (NHEJ) pathway or by homology-directed repair (HDR) pathway. The second is a mutant Cas9, known as the Cas9D10A, with only nickase activity, which means that it only cleaves one DNA strand and does not activate NHEJ. Thus, the DNA repairs proceed via the HDR pathway only. The third is a nuclease-deficient Cas9 (dCas9) which does not have cleavage activity but is able to bind DNA. Therefore, dCas9 is able to target specific sequences of a genome without cleavage. By fusing dCas9 with various effector domains, dCas9 can be used either as a gene silencing or activation tool.

Other gene-editing agents may also be used. For example, particular embodiments can utilize transcription activator-like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or by homologous recombination (HR) with an exogenous double-stranded donor DNA fragment.

As indicated, TALENs have been engineered to bind a target sequence and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12th and 13th positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

Examples of DNA cleavage domains that can be used in TALEN fusions are wild-type and variant Fokl endonucleases. The Fokl domain functions as a dimer requiring two constructs with unique DNA binding domains for sites on the target sequence. The Fokl cleavage domain cleaves within a five or six base pair spacer sequence separating the two inverted half-sites.

Particular embodiments can utilize MegaTALs as gene editing agents. MegaTALs have a single chain rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the MegaTAL only requires the delivery of a single peptide chain for functional activity.

Particular embodiments can utilize zinc finger nucleases (ZFNs) as gene editing agents. ZFNs are a class of site-specific nucleases engineered to bind and cleave DNA at specific positions. ZFNs are used to introduce DSBs at a specific site in a DNA sequence which enables the ZFNs to target unique sequences within a genome in a variety of different cells. Moreover, subsequent to DSB, homologous recombination or non-homologous end joining takes place to repair the DSB, thus enabling genome editing.

ZFNs are synthesized by fusing a zinc finger DNA-binding domain to a DNA cleavage domain. The DNA-binding domain includes three to six zinc finger proteins which are transcription factors. The DNA cleavage domain includes the catalytic domain of, for example, Fokl endonuclease.

Particular embodiments may also utilize interfering RNA-type mechanisms to down-regulate AIRE.

SEQ ID NOs: 1-4 (see FIG. 18) provide exemplary human and mouse AIRE protein and gene sequences. SEQ ID NO: 4 provides an exemplary AIRE gene sequence reflecting GenBank NC_000076.6. SEQ ID NOs: 5-8 provide exemplary human and mouse CD40 protein and gene sequences. Additional nucleic acid sequences encoding AIRE and CD40 proteins can be identified by those of ordinary skill in the art, and can include one or more of various sequence polymorphisms, mutations, and/or sequence variants (e.g., splice variants or codon optimized variants). Sequence information provided by public databases can be used to identify additional gene and protein sequences that can be used with the systems and methods disclosed.

Available prediction software can be used to generate guide RNA sequences to use in the aforementioned gene-editing methods. The utilized guide RNA sequences will be rare or unique in the genome to minimize or eliminate interaction with potential off-target sites. Particular embodiments can utilize the following two single guide RNA (sgRNA) sequences: AIREsg2 (Exon 1) 5′GCACCGCACCGAGATCGCGG(TGG)3′ (SEQ ID NO: 9) and AIRE sg3 (Exon 3) 5′ACCTAAACCAGTCCCGGAAA(GGG)3′ (SEQ ID NO: 10).

Embodiments disclosed herein include producing antibodies by exposing B cells with down-regulated AIRE function to antigens. Any antigen can be used. Particular examples include bacterial antigens, viral antigens, fungal antigens, and cancer antigens.

Exemplary bacterial antigens include anthrax protective antigen, lipopolysaccharides, toxin A (tcdA), toxin B (tcdB), capsular polysaccharides, diphtheria toxin, α-crystallin, mycolic acid, heat shock protein 65 (HSP65), hemagglutinin, pertactin, FIM2, FIM3, adenylate cyclase, pneumolysin, pneumococcal capsular polysaccharides, type 3 secretion system (T3SS), PcrV protein, Psl exopolysaccharide, rompA, α toxin, and tetanus toxin.

Exemplary viral antigens include envelope glycoprotein B, CMV pp65, EBV EBNAI, EBV P18, EBV P23, the S, M, and L proteins of hepatitis B virus, the pre-S antigen of hepatitis B virus, HBCAG DELTA, HBV HBE, hepatitis C viral RNA, HCV NS3, HCV NS4, HIV gp32, HIV gp41, HIV gp120, HIV gp160, HIV P17/24, HIV P24, HIV P55 GAG, HIV P66 POL, HIV TAT, HIV GP36, the Nef protein, hemagglutinin, neuraminidase, the measles virus fusion protein, rabies glycoprotein, rabies nucleoprotein, the respiratory syncytial viral fusion protein VP7sc, protein E1, and protein E2.

Exemplary fungal antigens include spherule antigens, capsular polysaccharides, heat shock protein 60 (HSP60), gp63, lipophosphoglycan, merozoite surface antigens, sporozoite surface antigens, circumsporozoite antigens, gametocyte/gamete surface antigens, the blood-stage antigen pf 155/RESA, glutathione-S-transferase, paramyosin, trichophytin, SAG-1, and p30.

Exemplary cancer antigens include CD19, CD20, CD33, CD133, ERBB2, GD2, HER2, mesothelin, PSCA, PSMA, ROR1, and WT1.

Particular embodiments disclosed herein include producing antibodies by exposing B cells with down-regulated AIRE function to antigens and adjuvants. An adjuvant refers to a material that enhances the immune response to an antigen. The precise mode of action is not understood for all adjuvants, but such lack of understanding does not prevent their use.

Any adjuvant can be used within the teachings of the current disclosure. Exemplary adjuvants include Toll-like receptor ligands, squalene-based adjuvants, alum, STING agonists, and cytokines.

Exemplary Toll-like receptor ligands include CpG, Cpg-28, Polyriboinosinic polyribocytidylic acid (Poly(I:C)), α-galactoceramide, MPLA, Motolimod, imiquimod, MGN1703, and Hiltonol.

Squalene is a triterpene that can be derived from certain plant sources, such as rice bran, wheat germ, amaranth seeds, and olives, as well as from animal sources, such as shark liver oil. Examples of squalene-based adjuvants include MF59® (Novartis, Basel, Switzerland) and Addavax™ (InvivoGen, San Diego, CA).

Alum refers to a family of salts that contain two sulfate groups, a monovalent cation, and a trivalent metal, such as aluminum or chromium. Alum is an FDA approved adjuvant.

“STING” is an abbreviation of “stimulator of interferon genes”. Exemplary STING agonists include c-AIMP; (3′,2′)c-AIMP; (2′,2′)c-AIMP; (2′,3′)c-AIMP; c-AIMP(S); c-(dAMP-dIMP); c-(dAMP-2′FdIMP); c-(2′FdAMP-2′FdIMP); (2′,3′)c-(AMP-2′FdIMP); c-[2′FdAMP(S)-2′FdIMP(S)]; c-[2′FdAMP(S)-2′FdIMP(S)](POM)2; and DMXAA.

Naturally occurring antibody structural units include a tetramer. Each tetramer includes two pairs of polypeptide chains, each pair having one light chain and one heavy chain.

The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific antigen epitope. From N-terminal to C-terminal, both light and heavy chain variable regions include the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989).

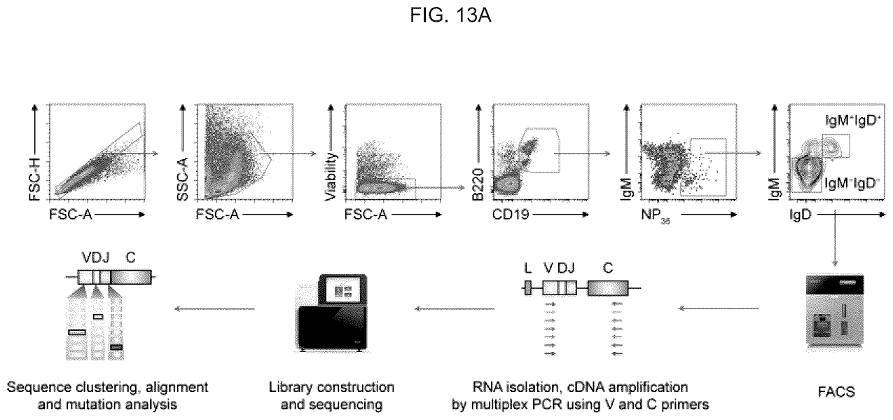

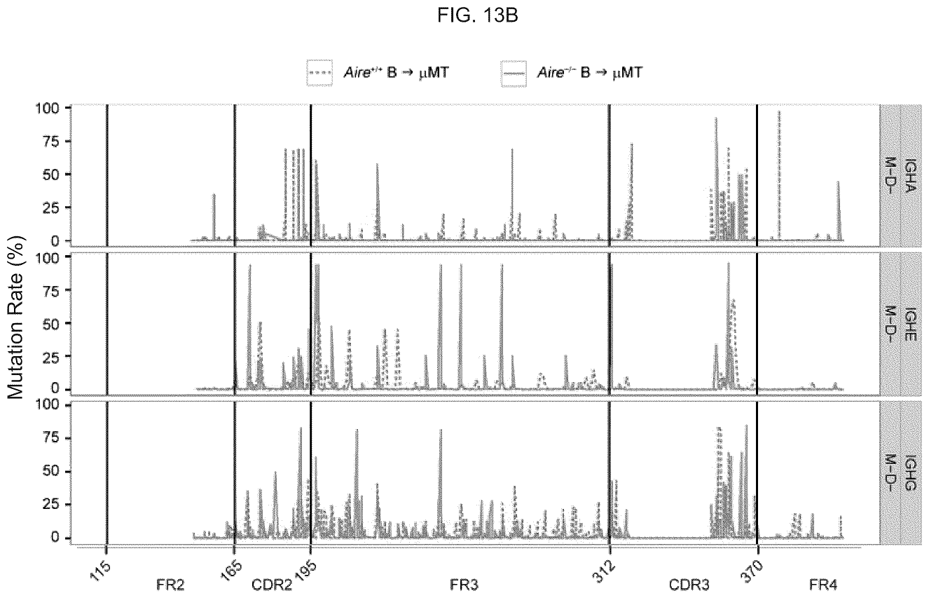

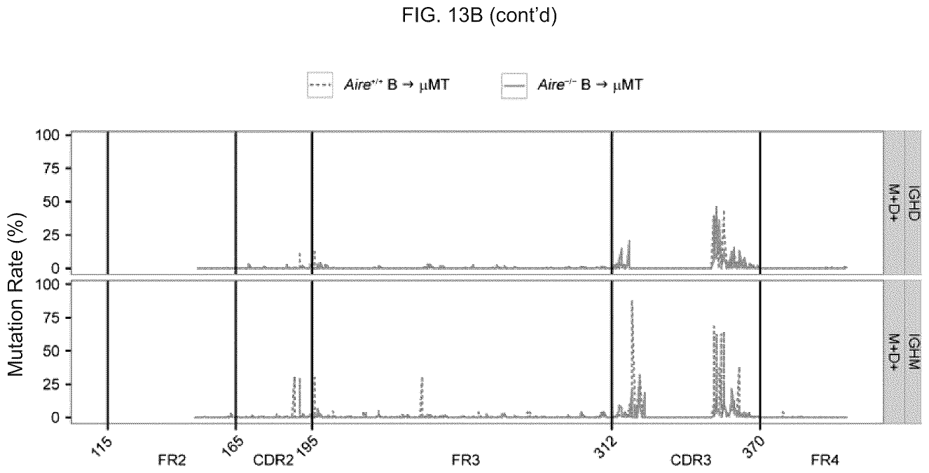

As indicated previously, SMH occurs in the CDR regions including the 3 heavy chain CDRs and the 3 light chain CDRs. Systems and methods disclosed herein can result in an increased mutation rate in CDR regions and/or FR regions (see, e.g., FIG. 13B). Thus, as used herein, increased SHM can include increased SHM in CDR regions and/or FR regions. An important feature of many neutralizing antibodies (e.g., HIV-1 neutralizing antibodies) is the increased number of mutations in FR regions (which is usually not mutated or poorly mutated during “regular” SHM (i.e., SHM in the absence of AIRE downregulation). Particular embodiments disclosed herein include producing antibodies with increased FR mutations.

The carboxy-terminal portion of each chain defines a constant region that can be responsible for effector function. Examples of effector functions include: C1q binding and complement dependent cytotoxicity (CDC); antibody-dependent cell-mediated cytotoxicity (ADCC); phagocytosis; down regulation of cell surface receptors (e.g. B cell receptors); and B cell activation.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgA is similarly subdivided into subclasses including IgA1 and IgA2. As indicated previously, CSR rearranges the immunoglobulin (Ig) heavy chain constant region genes from IgM or IgD to an IgG, IgA or IgE.

A human antibody is one which includes an amino acid sequence which corresponds to that of an antibody produced by a human or a human cell or derived from a non-human source that utilizes human antibody repertoires or other human antibody-encoding sequences.

Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008). Human antibodies may be prepared by administering an antigen to a transgenic animal that has been modified to produce intact human antibodies or intact antibodies with human variable regions in response to antigenic challenge. Such animals contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Traditional strategies for hybridoma development using mice, llamas, chicken, rats, hamsters, rabbits, etc. can also be used.

If produced antibodies are not human, such antibodies can be humanized. A “humanized” antibody refers to a chimeric antibody including amino acid residues from non-human CDRs and amino acid residues from human FRs. In particular embodiments, a humanized antibody will include substantially all of at least one, and typically two, variable domains, in which all or substantially all of the CDRs correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Nati. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

A human consensus framework is a framework which represents the most commonly occurring amino acid residues in a selection of human immunoglobulin variable light (V_{L}) or variable heavy (V_{H}) framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al., supra. In particular embodiments, for the V_{H}, the subgroup is subgroup III as in Kabat et al., supra.

Once antibodies have been generated, their CDRs can be identified. Definitive delineation of a CDR and identification of residues including the binding site of an antibody can be accomplished by solving the structure of the antibody and/or solving the structure of the antibody-antigen complex. In particular embodiments, this can be accomplished by methods such as X-ray crystallography.

CDRs from antibodies produced according to the methods disclosed herein can be utilized in a variety of binding domain formats. For example, particular embodiments can include binding fragments of an antibody, e.g., Fv, Fab, Fab′, F(ab′)₂, Fc, and single chain Fv fragments (scFvs) or any biologically effective fragments of an immunoglobulin that bind specifically to targeted antigen.

A single chain variable fragment (scFv) is a fusion protein of the variable regions of the heavy and light chains of immunoglobulins connected with a short linker peptide. Fv fragments include the V_{L} and V_{H} domains of a single arm of an antibody, but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird et al., Science 242 (1988) 423-426; Huston et al., Proc. Natl. Acad. Sci. USA 85 (1988) 5879-5883; Plueckthun, in The Pharmacology of Monoclonal Antibodies, vol. 113, Rosenburg and Moore (eds.), Springer-Verlag, New York), (1994) 269-315; WO1993/16185; U.S. Pat. No. 5,571,894; and U.S. Pat. No. 5,587,458.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117, 4542-51, 2011)) can also be formed. Antibody fragments can also include isolated CDRs. For a review of antibody fragments, see Hudson et al., Nat. Med. 9:129-134, 2003.

Unless otherwise indicated, the term “antibody” includes antibodies including two full-length heavy chains and two full-length light chains, the fragments as described above, and variants. Furthermore, unless explicitly excluded, antibodies can include monoclonal antibodies, human or humanized antibodies, bispecific antibodies, polyclonal antibodies, linear antibodies, minibodies, domain antibodies, synthetic antibodies, chimeric antibodies, antibody fusions, and fragments thereof, respectively.

A monoclonal antibody refers to an antibody obtained from a population of substantially homogeneous antibodies, i.e., the individual antibodies including the population are identical and/or bind the same antigen epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes of an antigen, each monoclonal antibody of a monoclonal antibody preparation is directed against a single epitope on an antigen. Thus, the modifier “monoclonal” indicates the character of the antibody as being obtained from a substantially homogeneous population of antibodies, and is not to be construed as requiring production of the antibody by any particular method. For example, monoclonal antibodies can be made by a variety of techniques, including the hybridoma method, recombinant DNA methods, and methods utilizing transgenic animals containing all or part of the human immunoglobulin loci.

Variants of antibodies can include those having one or more conservative amino acid substitutions or one or more non-conservative substitutions that do not adversely affect the binding of the antibody.

In particular embodiments, a conservative amino acid substitution may not substantially change the structural characteristics of the reference antibody (e.g., a replacement amino acid should not tend to break a helix that occurs in the reference sequence, or disrupt other types of secondary structure that characterizes the reference sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N.Y. (1991)); and Thornton et al., Nature, 354:105 (1991).

In particular embodiments, a “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gln); Group 4: Arginine (Arg), Lysine (Lys), Histidine (His); Group 5: Isoleucine (Ile), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

In particular embodiments, a V_{L} region can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared to an antibody produced and characterized according to methods disclosed herein. An insertion, deletion or substitution may be anywhere in the V_{L} region, including at the amino- or carboxy-terminus or both ends of this region, provided that each CDR includes zero changes or at most one, two, or three changes and provided an antibody including the modified V_{L} region can still specifically bind its target antigen with an affinity similar to the reference antibody.

In particular embodiments, a V_{H} region can be derived from or based on a disclosed V_{H} and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with when compared to an antibody produced and characterized according to methods disclosed herein. An insertion, deletion or substitution may be anywhere in the V_{H} region, including at the amino-or carboxy-terminus or both ends of this region, provided that each CDR includes zero changes or at most one, two, or three changes and provided an antibody including the modified V_{H} region can still specifically bind its target epitope with an affinity similar to the reference antibody.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions.

In particular embodiments, variants have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-live.

Antibodies produced according to the methods disclosed herein have high affinity for their target antigens. In particular embodiments “affinity” refers to the strength of the sum total of noncovalent interactions between a single binding site of an antibody and its target antigen. Unless indicated otherwise, “binding affinity” refers to intrinsic binding affinity which reflects a 1:1 interaction between members of a binding pair (i.e., antibody and target antigen). The affinity of an antibody for its target antigen can generally be represented by the dissociation constant (Kd) or the association constant (K_{A}). Affinity can be measured by common methods known in the art.

In particular embodiments, binding affinities can be assessed in relevant in vitro conditions, such as a buffered salt solution approximating physiological pH (7.4) at room temperature or 37° C.

In particular embodiments, “high affinity” means that the antibody associates with its target antigen with a dissociation constant (1(D) of 10⁻⁸ M or less, in particular embodiments of from 10⁻⁵ M to 10⁻¹³ M, in particular embodiments of from 10⁻⁵ M to 10⁻¹⁰ M, in particular embodiments of from 10⁻⁵ M to 10⁻⁷ M, in particular embodiments of from 10⁻⁸ M to 10⁻¹³ M, or in particular embodiments of from 10⁻⁹ M to 10⁻¹³ M. The term can be further used to indicate that the antibody does not bind to other biomolecules present, (e.g., it binds to other biomolecules with a dissociation constant (KD) of 10⁻⁴ M or more, in particular embodiments of from 10⁻⁴ M to 1 M).

In particular embodiments, “high affinity” means that the antibody associates with its target antigen with an affinity constant (i.e., association constant, K_{A}) of 10⁷ M⁻¹ or more, in particular embodiments of from 10⁵ M⁻¹ to 10¹³ M⁻¹, in particular embodiments of from 10⁵ M⁻¹ to 10¹⁰ M⁻¹, in particular embodiments of from 10⁵ M⁻¹ to 10⁸ M⁻¹, in particular embodiments of from 10⁷ M⁻¹ to 10¹³ M⁻¹, or in particular embodiments of from 10⁷ M⁻¹ to 10⁸ M⁻¹. The term can be further used to indicate that the antibody does not bind to other biomolecules present, (e.g., it binds to other biomolecules with an association constant (K_{A}) of 10⁴ M⁻¹ or less, in particular embodiments of from 10⁴ M⁻¹ to 1 M⁻¹).

In particular embodiments, “high affinity” is relative to an antibody produced in the absence of AIRE down-regulation.

In particular embodiments, developed antibodies can be produced from a gene using a protein expression system. Protein expression systems can utilize DNA constructs (e.g., chimeric genes, expression cassettes, expression vectors, recombination vectors) including a nucleic acid sequence encoding the protein or proteins of interest operatively linked to appropriate regulatory sequences. In particular embodiments, such DNA constructs are not naturally occurring DNA molecules and are useful for introducing DNA into host-cells to express selected proteins of interest. In particular embodiments, a DNA construct that encodes an antibody can be inserted into cells (e.g., bacterial, mammalian, insect, etc.), which can produce the antibody encoded by the DNA construct.

Operatively linked refers to the linking of DNA sequences (including the order of the sequences, the orientation of the sequences, and the relative spacing of the various sequences) in such a manner that the encoded protein is expressed. Methods of operatively linking expression control sequences to coding sequences are well known in the art. See, e.g., Maniatis et al., Molecular Cloning: A Laboratory Manual, Cold Spring Harbor, N. Y., 1982; and Sambrook et al., Molecular Cloning: A Laboratory Manual, Cold Spring Harbor, N. Y., 1989.

Expression control sequences are DNA sequences involved in any way in the control of transcription or translation. Suitable expression control sequences and methods of making and using them are well known in the art. Expression control sequences generally include a promoter. The promoter may be inducible or constitutive. It may be naturally occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-2361, 1987. Also, the location of the promoter relative to the transcription start may be optimized. See, e.g., Roberts et al., Proc. Natl. Acad. Sci. USA, 76:760-764, 1979.

The promoter may include, or be modified to include, one or more enhancer elements. In particular embodiments, the promoter will include a plurality of enhancer elements. Promoters including enhancer elements can provide for higher levels of transcription as compared to promoters that do not include them.

For efficient expression, the coding sequences can be operatively linked to a 3′ untranslated sequence. In particular embodiments, the 3′ untranslated sequence can include a transcription termination sequence and a polyadenylation sequence. The 3′ untranslated region can be obtained, for example, from the flanking regions of genes.

In particular embodiments, a 5′ untranslated leader sequence can also be employed. The 5′ untranslated leader sequence is the portion of an mRNA that extends from the 5′ CAP site to the translation initiation codon.

In particular embodiments, a “hisavi” tag can be added to the N-terminus or C-terminus of a gene by the addition of nucleotides coding for the Avitag amino acid sequence, “GLNDIFEAQKIEWHE” (SEQ ID NO: 11), as well as the 6x histidine tag coding sequence “HHHHHH (SEQ ID NO: 12)”. The Avitag avidity tag can be biotinylated by a biotin ligase to allow for biotin-avidin or biotin-streptavidin based interactions for protein purification, as well as for immunobiology (such as immunoblotting or immunofluorescence) using anti-biotin antibodies. The 6x histidine tag allows for protein purification using Ni-²⁺ affinity chromatography.

In particular embodiments, the DNA constructs can be introduced by transfection, a technique that involves introduction of foreign DNA into the nucleus of eukaryotic cells. In particular embodiments, the proteins can be synthesized by transient transfection (DNA does not integrate with the genome of the eukaryotic cells, but the genes are expressed for 24-96 hours). Various methods can be used to introduce the foreign DNA into the host-cells, and transfection can be achieved by chemical-based means including by the calcium phosphate, by dendrimers, by liposomes, and by the use of cationic polymers. Non-chemical methods of transfection include electroporation, sono-poration, optical transfection, protoplast fusion, impalefection, and hydrodynamic delivery. In particular embodiments, transfection can be achieved by particle-based methods including gene gun where the DNA construct is coupled to a nanoparticle of an inert solid which is then “shot” directly into the target-cell's nucleus. Other particle-based transfection methods include magnet assisted transfection and impalefection.

### EXEMPLARY EMBODIMENTS

### Exemplary Embodiments—Set 1

1. A method of increasing somatic hypermutation (SHM) and class switch recombination (CSR) during antibody production including selecting a first population of B cells with down-regulated autoimmune regulator (AIRE) function; and contacting the first selected B cell population with an antigen; thereby increasing SHM and CSR during antibody production wherein the increase is in relation to antibodies produced by selecting a second population of B cells with normal AIRE function; and contacting the second selected B cell population with the same antigen under comparable conditions.

2. A method of embodiment 1 further including contacting the first selected B cell population and the second selected B cell population with an adjuvant.

3. A method of embodiment 1 or 2 further including isolating the produced antibodies with increased SHM and CSR.

4. A method of any of embodiments 1-3 further including determining the CDR sequences of the produced antibodies with increased SHM and CSR.

5. A method of any of embodiments 1-4 further including modifying B cells to down-regulate AIRE function.

6. A method of embodiment 5 wherein the modifying produces the selected population of B cells of embodiment 1.

7. A method of embodiment 5 or 6 wherein the modifying includes AIRE gene editing and/or CD40 gene editing.

8. A method of embodiment 7 wherein the AIRE gene editing and/or CD40 gene editing includes CRISPR-Cas gene editing, transcription activator like effector nuclease (TALEN) gene editing, MegaTal gene editing, or zinc finger nuclease (ZFN) gene editing.

9. A method of any of embodiments 5-8 wherein the AIRE gene editing includes contacting the modified B cells with SEQ ID NO: 9 and SEQ ID NO: 10.

10. A method of any of embodiments 5-8 wherein the modifying results in AIRE protein that does not interact with AID.

11. A method of embodiment 10 wherein the AIRE protein lacks its caspase activation and recruitment domain (CARD) and/or its nuclear localization signal (NLS).

12. A method of embodiment 10 wherein the AIRE protein lacks amino acids 110-114 and 131-133 or lacks amino acids 101-180.

13. A method of any of embodiments 1-12 wherein the selected first and second B cell populations are within different but comparable in vitro culture conditions.

14. A method of embodiment 13 further including stimulating the first and second B cell populations within the in vitro culture conditions.

15. A method of embodiment 14 wherein the stimulating includes adding CD40L, IL-4, IFN-y or TGF-β

16. A method of any of embodiments 1-12 wherein the selected first and second B cell populations are in vivo in a subject.

17. A method of embodiment 16 wherein the in vivo selected B cell populations are within a mouse, llama, chicken, rat, hamster, or rabbit.

18. A method of embodiment 16 or 17 further including administering the selected first population of B cells to the subject.

19. A method of embodiment 18 wherein the selected first population of B cells are AIRE^{-/-}.

20. A method of embodiment 17 wherein the mouse produces human antibodies.

21. A method of any of embodiments 1-20 wherein the produced antibodies are human antibodies.

22. A method of any of embodiments 1-20 wherein the produced antibodies are non-human antibodies.

23. A method of embodiment 22 further including humanizing the produced antibodies.

24. A method of any of embodiments 1-23 wherein the antigen is a viral antigen, a bacterial antigen, a fungal antigen, or a cancer antigen.

25. A method of any of embodiments 2-24 wherein the adjuvant is a Toll-like receptor ligand, a squalene-based adjuvant, alum, a STING agonist, and/or a cytokine.

26. A method of any of embodiments 1-25 wherein the increased SHM is within CDR regions, FR regions, or CDR regions and FR regions.

### Exemplary Embodiments—Set 2

1. A method of producing a neutralizing antibody including selecting a population of B cells with down-regulated autoimmune regulator (AIRE) function; and contacting the selected B cell population with an antigen; thereby producing a neutralizing antibody.

2. A method of embodiment 1 further including contacting the selected B cell population with an adjuvant.

3. A method of embodiment 1 or 2 further including isolating the neutralizing antibody.

4. A method of any of embodiments 1-3 further including determining the CDR sequences of the neutralizing antibody.

5. A method of any of embodiments 1-4 further including modifying B cells to down-regulate AIRE function.

6. A method of embodiment 5 wherein the modifying produces the selected population of B cells of embodiment 1.

7. A method of embodiment 5 or 6 wherein the modifying includes AIRE gene editing and/or CD40 gene editing.

8. A method of embodiment 7 wherein the AIRE gene editing and/or CD40 gene editing includes CRISPR-Cas gene editing, transcription activator like effector nuclease (TALEN) gene editing, MegaTal gene editing, or zinc finger nuclease (ZFN) gene editing.

9. A method of any of embodiments 5-8 wherein the AIRE gene editing includes contacting the modified B cells with SEQ ID NO: 9 and SEQ ID NO: 10.

10. A method of any of embodiments 5-8 wherein the modifying results in AIRE protein that does not interact with AID.

11. A method of embodiment 10 wherein the AIRE protein lacks its caspase activation and recruitment domain (CARD) and/or its nuclear localization signal (NLS).

12. A method of embodiment 10 wherein the AIRE protein lacks amino acids 110-114 and 131-133 or lacks amino acids 101-180.

13. A method of any of embodiments 1-12 wherein the selected B cell population is within in vitro culture conditions.

14. A method of embodiment 13 further including stimulating the B cell population within the in vitro culture conditions.

15. A method of embodiment 14 wherein the stimulating includes adding CD40L, IL-4, IFN-y or TGF-β

16. A method of any of embodiments 1-12 wherein the selected B cell population is in vivo in a subject.

17. A method of embodiment 16 wherein the in vivo selected B cell population is within a mouse, llama, chicken, rat, hamster, or rabbit.

18. A method of embodiment 16 or 17 further including administering the selected B cell population to the subject.

19. A method of embodiment 18 wherein the selected B cell population is AIRE^{-/-}.

20. A method of embodiment 17 wherein the mouse produces a human neutralizing antibody.

21. A method of any of embodiments 1-20 wherein the produced neutralizing antibody is a human neutralizing antibody.

22. A method of any of embodiments 1-20 wherein the produced neutralizing antibody is a non-human neutralizing antibody.

23. A method of embodiment 22 further including humanizing the produced neutralizing antibody.

24. A method of any of embodiments 1-23 wherein the antigen is a viral antigen, a bacterial antigen, a fungal antigen, or a cancer antigen.

25. A method of any of embodiments 2-24 wherein the adjuvant is a Toll-like receptor ligand, a squalene-based adjuvant, alum, a STING agonist, and/or a cytokine.

### Exemplary Embodiments—Set 3

1. A method of producing antibodies with high affinity for an antigen including selecting a population of B cells with down-regulated autoimmune regulator (AIRE) function; and contacting the selected B cell population with the antigen; thereby producing antibodies with high affinity for the antigen.

2. A method of embodiment 1 further including contacting the selected B cell population with an adjuvant.

3. A method of embodiment 1 or 2 further including isolating the produced antibodies.

4. A method of any of embodiments 1-3 further including determining the CDR sequences of the produced antibodies.

5. A method of any of embodiments 1-4 further including modifying B cells to down-regulate AIRE function.

6. A method of embodiment 5 wherein the modifying produces the selected population of B cells of embodiment 1.

7. A method of embodiment 5 or 6 wherein the modifying includes AIRE gene editing and/or CD40 gene editing.

8. A method of embodiment 7 wherein the AIRE gene editing and/or CD40 gene editing includes CRISPR-Cas gene editing, transcription activator like effector nuclease (TALEN) gene editing, MegaTal gene editing, or zinc finger nuclease (ZFN) gene editing.

9. A method of any of embodiments 5-8 wherein the AIRE gene editing includes contacting the modified B cells with SEQ ID NO: 9 and SEQ ID NO: 10.

10. A method of any of embodiments 5-9 wherein the modifying results in AIRE protein that does not interact with AID.

11. A method of embodiment 10 wherein the AIRE protein lacks its caspase activation and recruitment domain (CARD) and/or its nuclear localization signal (NLS).

12. A method of embodiment 11 wherein the AIRE protein lacks amino acids 110-114 and 131-133 or lacks amino acids 101-180.

13. A method of any of embodiments 1-13 wherein the selected B cell population is within an in vitro culture.

14. A method of embodiment 13 further including stimulating the B cell population within thein vitro culture conditions.

15. A method of embodiment 14 wherein the stimulating includes adding CD40L, IL-4, IFN-y or TGF-β.

16. A method of any of embodiments 1-15 wherein the selected B cell population is in vivo.

17. A method of embodiment 16 wherein the in vivo selected B cell populations are within a mouse, llama, chicken, rat, hamster, or rabbit.

18. A method of embodiment 16 or 17 further including administering the selected first population of B cells to the subject.

19. A method of embodiment 18 wherein the selected first population of B cells are AIRE^{-/-}.

20. A method of embodiment 17 wherein the mouse produces human antibodies.

21. A method of any of embodiments 1-20 wherein the produced antibodies are human antibodies.

22. A method of any of embodiments 1-21 wherein the produced antibodies are non-human antibodies.

23. A method of embodiment 22 further including humanizing the produced antibodies.

24. A method of any of embodiments 1-23 wherein the antigen is a viral antigen, a bacterial antigen, a fungal antigen, or a cancer antigen.

25. A method of any of embodiments 2-24 wherein the adjuvant is a Toll-like receptor ligand, a squalene-based adjuvant, alum, a STING agonist, and/or a cytokine.

### Exemplary Embodiments—Set 4

1. A kit for antibodies including: a B cell population with down-regulated AIRE function; and an antigen.

2. A kit of embodiment 1 further including an adjuvant.

3. A kit of embodiment 1 or 2 further including CD40L, IL-4, IFN-y and/or TGF-β.

4. A kit of any of embodiments 1-3 wherein the B cell population is in vitro.

5. A kit of any of embodiments 1-3 wherein the B cell population is in vivo.

6. A kit of any of embodiments 1-3 including an in vitro B cell population with down-regulated AIRE function and an in vivo B cell population with down-regulated AIRE function.

7. A kit of any of embodiments 1-6 wherein the B cell population is AIRE^{-/-}.

8. A kit of any of embodiments 1-6 wherein the B cell population expresses an AIRE protein that does not interact with AID.

9. A kit of embodiment 8 wherein the AIRE protein lacks its caspase activation and recruitment domain (CARD) and/or its nuclear localization signal (NLS).

10. A kit of embodiment 9 wherein the AIRE protein lacks amino acids 110-114 and 131-133 or lacks amino acids 101-180.

11. A kit of embodiment 5 or 6 wherein the in vivo B cell population is within a mouse, llama, chicken, rat, hamster, or rabbit.

12. A kit of embodiment 11 wherein the mouse produces human antibodies.

13. A kit of any of embodiments 1-12 wherein the antigen is a viral antigen, a bacterial antigen, a fungal antigen, or a cancer antigen.

14. A kit of any of embodiments 2-13 wherein the adjuvant is a Toll-like receptor ligand, a squalene-based adjuvant, alum, a STING agonist, and/or a cytokine.

### Exemplary Embodiments—Set 5

1. A kit for producing antibodies including: a B cell population; gene editing agents to down-regulate AIRE function and/or CD40 function in the B cell population; and an antigen.

2. A kit of embodiment 1 further including an adjuvant.

3. A kit of embodiment 1 or 2 further including CD40L, IL-4, IFN-y and/or TGF-β.

4. A kit of any of embodiments 1-3 wherein the B cell population is in vitro.

5. A kit of any of embodiments 1-3 wherein the B cell population is in vivo.

6. A kit of any of embodiments 1-3 including an in vitro B cell population with down-regulated AIRE function and an in vivo B cell population with down-regulated AIRE function.

7. A kit of any of embodiments 1-6 wherein the B cell population is AIRE^{-/-}.

8. A kit of any of embodiments 1-6 wherein the B cell population expresses an AIRE protein that does not interact with AID.

9. A kit of embodiment 8 wherein the AIRE protein lacks its caspase activation and recruitment domain (CARD) and/or its nuclear localization signal (NLS).

10. A kit of embodiment 9 wherein the AIRE protein lacks amino acids 110-114 and 131-133 or lacks amino acids 101-180.

11. A kit of embodiment 5 or 6 wherein the in vivo B cell population is within a mouse, llama, chicken, rat, hamster, or rabbit.

12. A kit of embodiment 11 wherein the mouse produces human antibodies.

13. A kit of any of embodiments 1-12 wherein the antigen is a viral antigen, a bacterial antigen, a fungal antigen, or a cancer antigen.

14. A kit of any of embodiments 2-13 wherein the adjuvant is a Toll-like receptor ligand, a squalene-based adjuvant, alum, a STING agonist, and/or a cytokine.

15. A kit of any of embodiments 1-14 wherein the AIRE gene editing and/or CD40 gene editing includes CRISPR-Cas gene editing, transcription activator like effector nuclease (TALEN) gene editing, MegaTal gene editing, or zinc finger nuclease (ZFN) gene editing.

16. A kit of any of embodiments 1-15 wherein the gene editing agents include SEQ ID NO: 9 and/or SEQ ID NO: 10.

Also provided is use of a method or kit of any of the preceding embodiments to increase SHM mutations in the FR regions of antibodies.

### Example 1. AIRE Inhibits AID-Mediated Antibody Diversification in Germinal Centre B Cells and Limits Autoimmunity.

Example 1 shows that AIRE is expressed in GC B cells in a CD40-dependent manner, interacts via its caspase activation and recruitment domain (CARD) and nuclear localization signal (NLS) with AID, and negatively regulates AID-mediated antibody diversification. AIRE-deficient mouse B cells undergo elevated CSR and affinity maturation after antigenic stimulation, which correlates with enhanced generation of genomic uracil, elevated Ig SHM, augmented AID targeting to Ig switch regions and increased interaction of AID with transcriptionally stalled RNA polymerase II (Pol II). Consistently, naive B cells of APS-1 patients undergo increased CSR upon stimulation ex vivo. Mice with AIRE deficiency in B cells have elevated levels of autoantibodies against T helper 17 (T_{H}17) effector cytokines and heightened skin C. albicans burden after infection, which recapitulates APS-1 patients. The disclosed results define a previously unknown but crucial B cell-intrinsic AIRE-dependent GC checkpoint of antibody diversification that limits autoimmunity, and illuminate new approaches of generating high-affinity neutralizing antibodies for therapeutic, diagnostic and research applications.

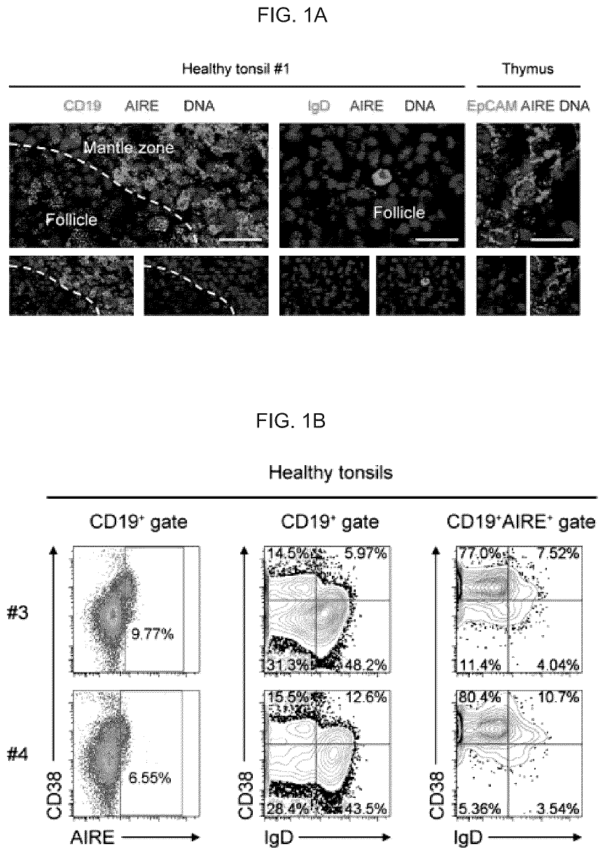

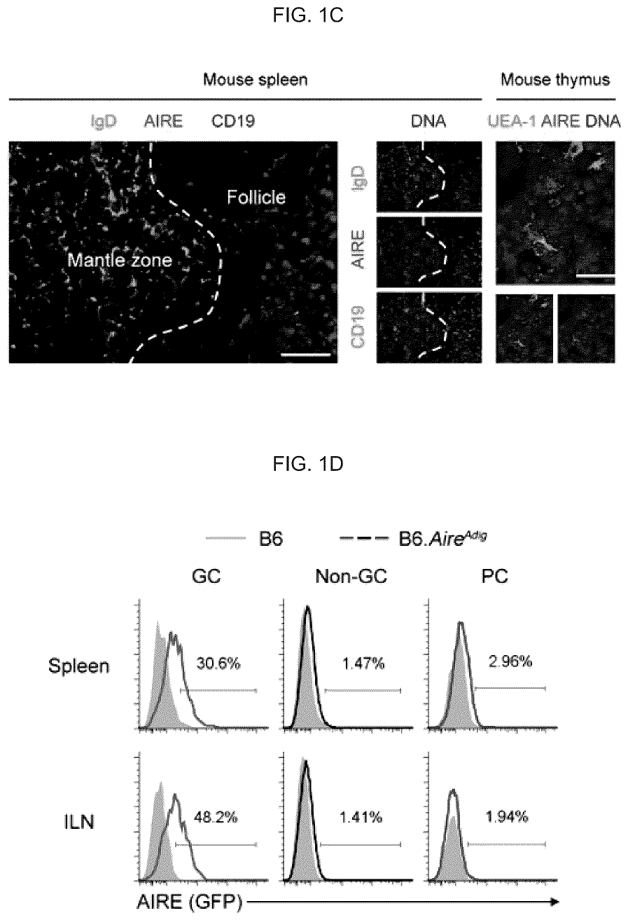

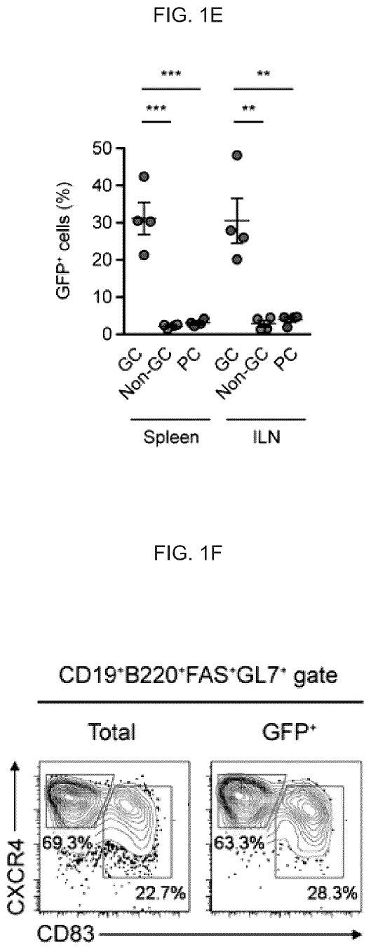

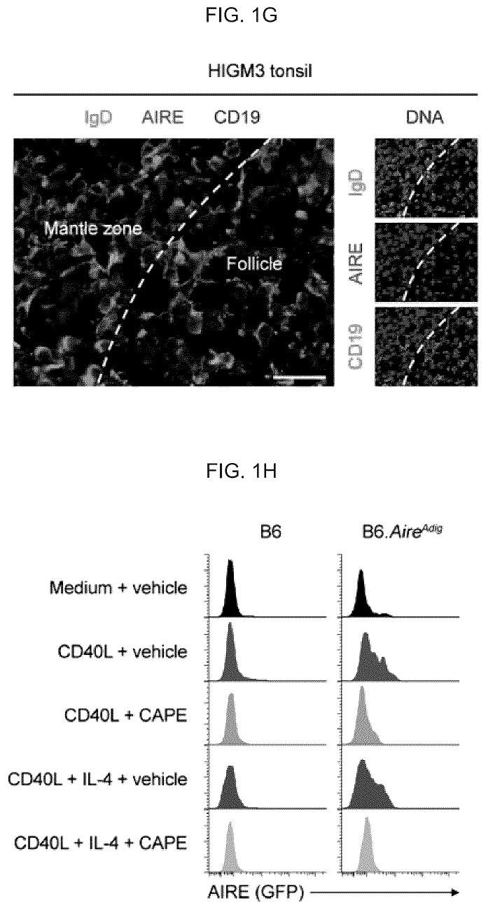

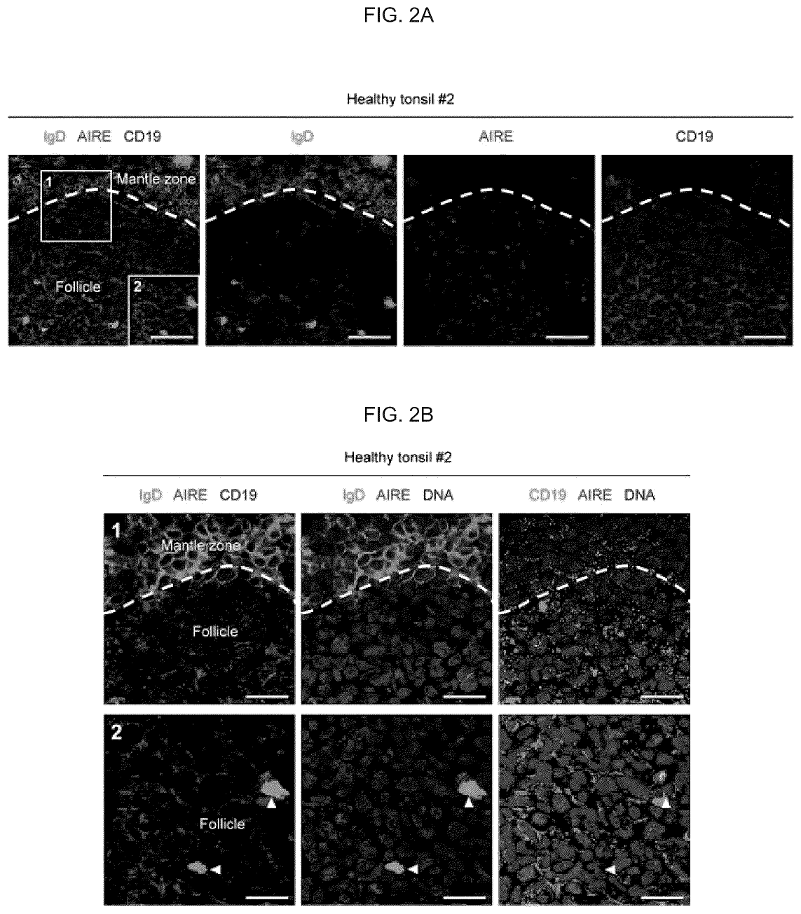

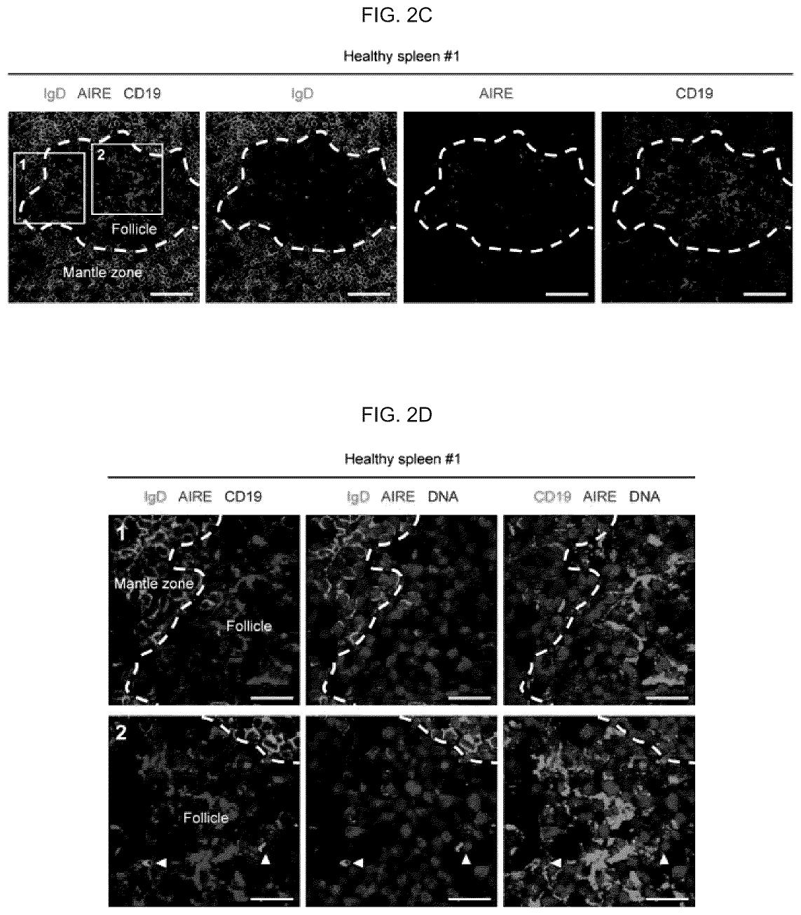

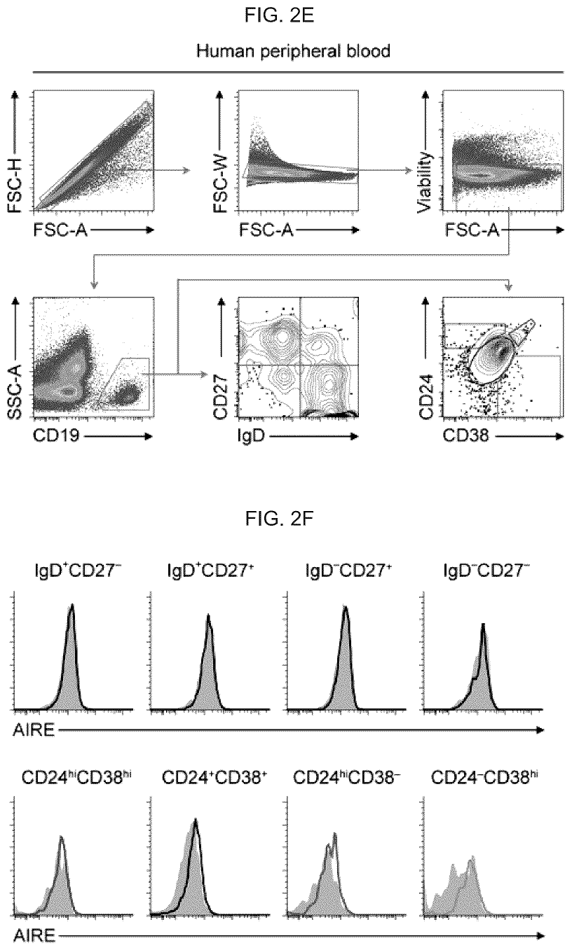

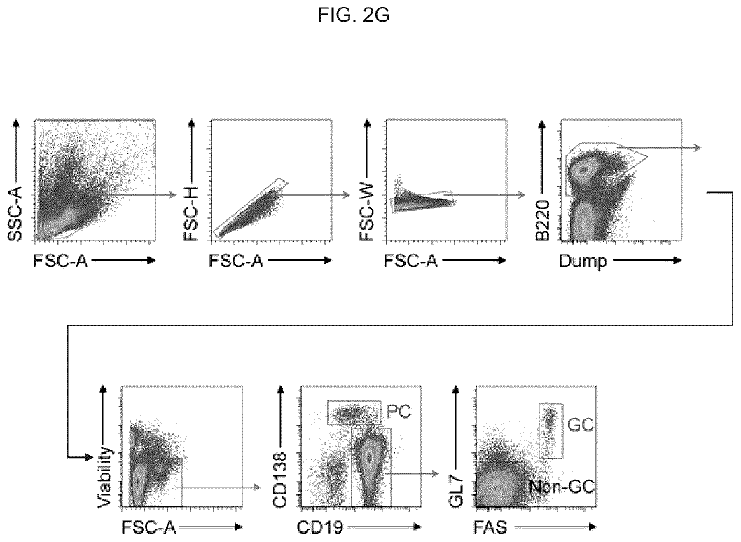

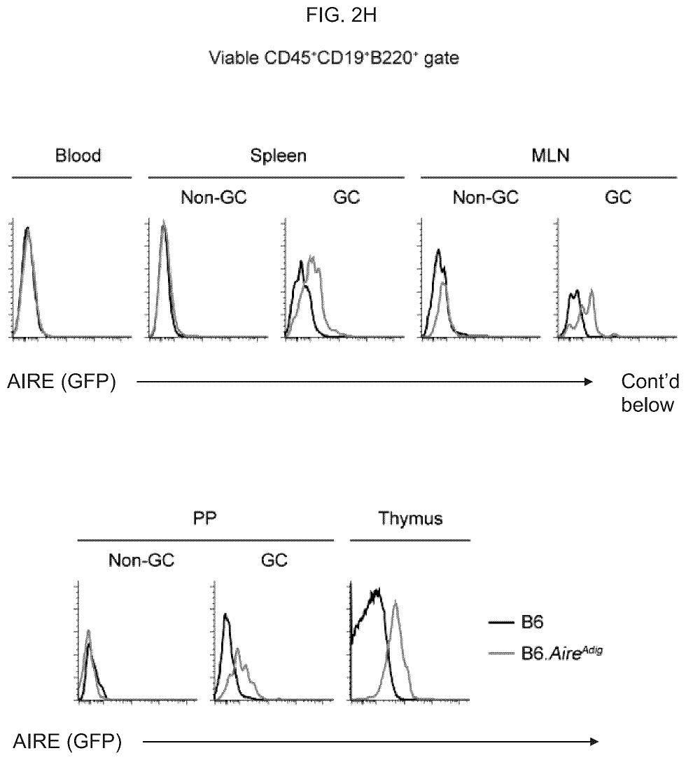

AIRE is essential to central and peripheral T cell tolerance, which consequently regulates humoral immunity. Anderson et al., Science 298, 1395-1401, (2002); Gardner et al., Science 321, 843-847, (2008); Malchow et al., Science 339, 1219-1224, (2013). To determine whether AIRE has a B cell-intrinsic function in humoral immunity, AIRE expression in B cells of human secondary lymphoid tissues was examined by immunofluorescence using an antibody that detects AIRE in the nuclei of thymic medullary epithelial cells (mTECs) (FIG. 1A). IgD⁻ B cells were identified inside tonsillar and splenic follicles that harbored nuclear AIRE (FIG. 1A, FIGS. 2A-2D). In contrast, tonsillar IgD⁺ B cells in the mantle zone and IgD⁺ plasmablasts in GCs and extrafollicular areas (Chen et al., Nature immunology 10, 889-898, (2009)) expressed little or no AIRE (FIG. 1A, FIGS. 2A-2D). Peripheral blood IgD⁺CD27⁻ or CD24⁺CD38^{Io} naive, IgD⁺CD27⁺ circulating marginal zone, IgD⁻CD27⁺ or CD24^{hi}CD38⁻ memory, IgD⁻CD27⁻ atypical memory and CD24^{hi}CD38^{hi} transitional B cells as well as CD24⁻CD38^{hi} plasma cells (PCs) did not express AIRE either (FIGS. 2E, 2F). Consistent with their follicular localization, tonsillar AIRE⁺ B cells were mostly IgD⁻CD38⁺ GC B cells (FIG. 1B). AIRE expression was similarly found in B cells in the splenic follicles of immunized mice (FIG. 1C). In the Aire^{Adig} reporter mice (Gardner et al., Science 321, 843-847, (2008)), B cell AIRE expression was detected in FAS⁺GL7⁺ GC B cells in the spleen, inguinal lymph nodes (ILNs), mesenteric lymph nodes (MLNs) and Peyer's patches (PPs) and in thymic B cells, but not in FAS⁻GL7⁻ non-GC B cells or CD138⁺ PCs in these tissues or in peripheral blood B cells (FIGS. 1D, 1E, FIGS. 2G, 2H), and there was no preferential distribution of AIRE in CXCR4⁺CD83⁻ dark zone (DZ) vs. CXCR4^{Io}CD83⁺ light zone (LZ) B cells (FIG. 1F). These data indicate that AIRE expression in GC B cells is a conserved characteristic of human and mouse secondary lymphoid tissues.

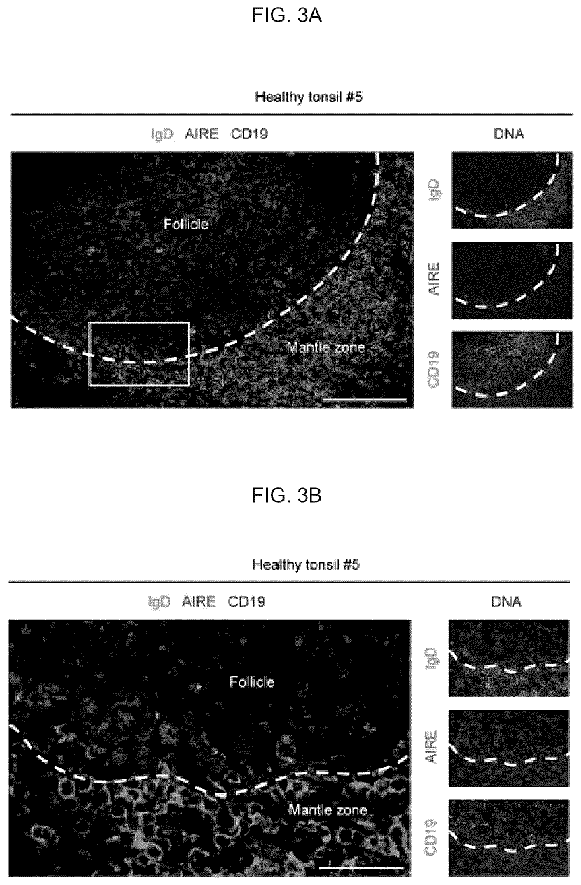

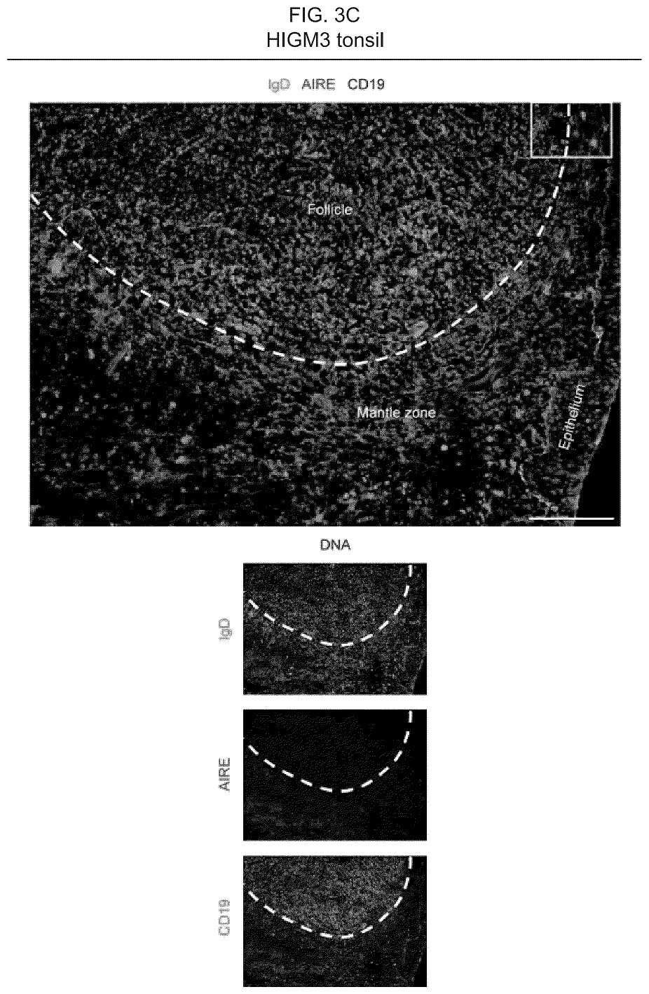

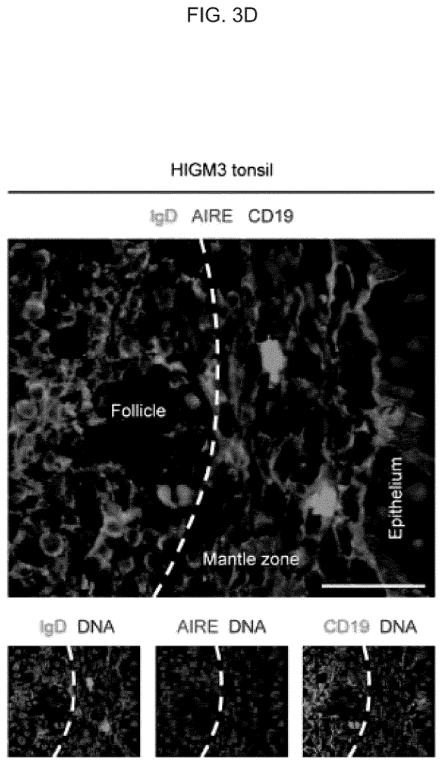

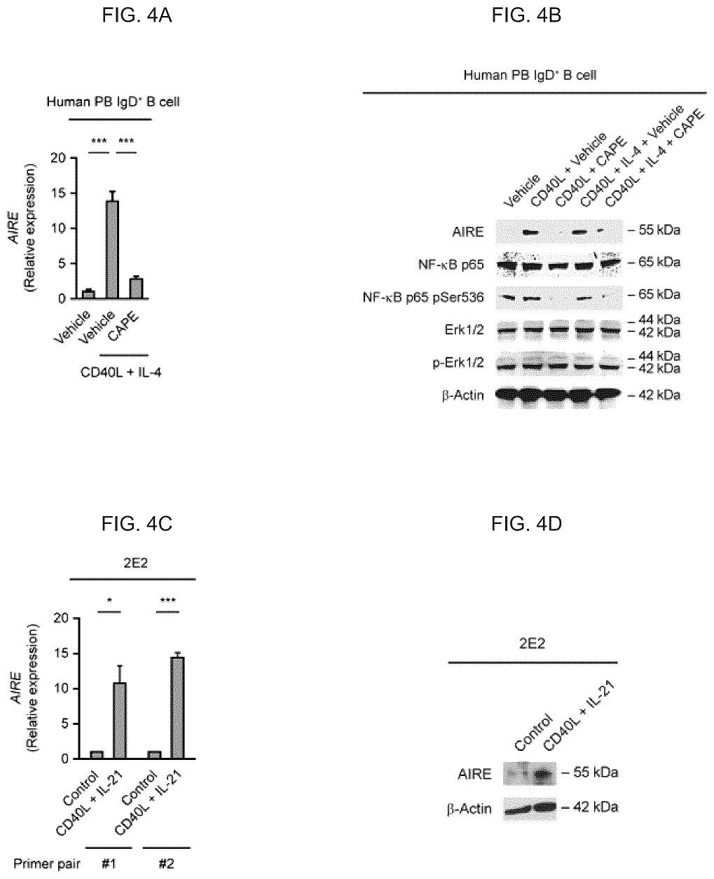

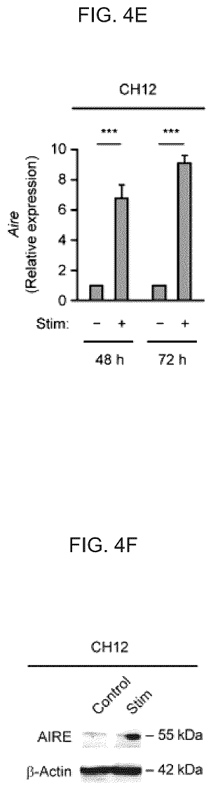

It was further sought to identify the regulation of GC B cell AIRE expression and examined the role of CD40 signalling, which is critical for T cell-dependent GC B cell responses (Liu et al., Nature 342, 929-931, (1989)) and was previously reported to promote AIRE expression by mTECs and thymic B cells. Akiyama et al., Immunity 29, 423-437, (2008); Yamano et al., Immunity 42, 1048-1061, (2015). In contrast to the prominent AIRE expression in tonsillar follicular B cells of healthy subjects (FIG. 1A, FIGS. 2A, 2B, FIGS. 3A, 3B), tonsillar follicular B cells of a patient with the rare primary immunodeficiency hyper-IgM syndrome type 3 (HIGM3), which is caused by loss-of-function mutations in the CD40 gene (Durandy et al., Immunological reviews 203, 67-79, (2005)), did not express AIRE (FIG. 1G, FIGS. 3C, 3D). AIRE mRNA and protein levels were induced in human peripheral blood IgD⁺ B cells upon stimulation with CD40 ligand (CD40L) alone or with IL-4, which was inhibited by caffeic acid phenethyl ester (CAPE), a selective inhibitor of nuclear factor-kappa B (NF-_{K}B) (Natarajan et al.,Proceedings of the National Academy of Sciences of the United States of America 93, 9090-9095, (1996)), the transcription factor activated by CD40 (FIGS. 4A, 4B). Similarly, mouse splenic B cells expressed AIRE upon CD40L stimulation ex vivo, which was abrogated by CAPE (FIG. 1H). In addition, human 2E2 and mouse CH12 cells, two B cell lines that undergo CD40L-induced CSR in vitro, had increased AIRE mRNA and protein expression upon CD40 ligation (FIGS. 4C-4F). Therefore, CD40 signalling is required for AIRE expression in GC B cells in vivo and promotes AIRE expression by B cells in vitro.

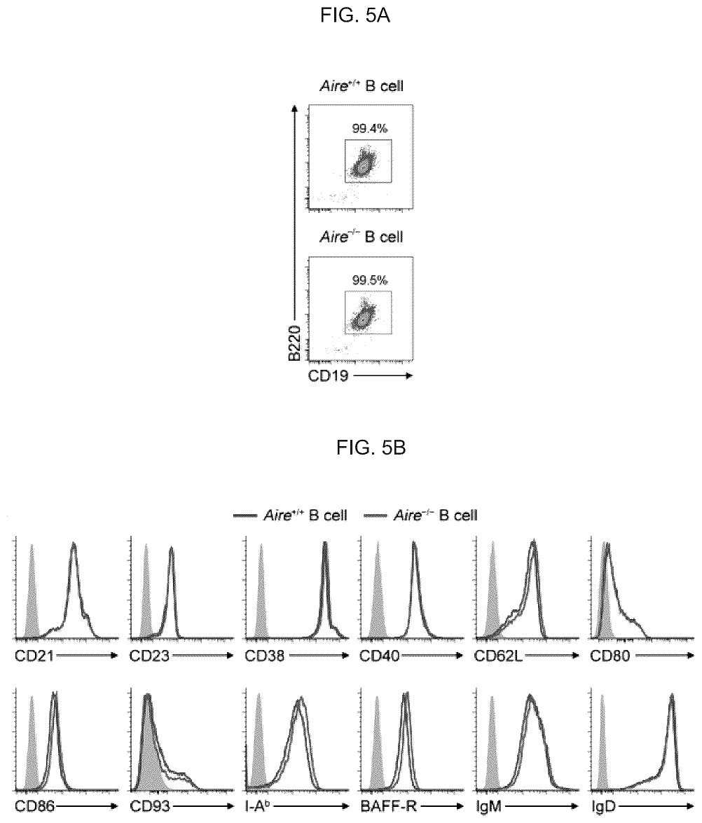

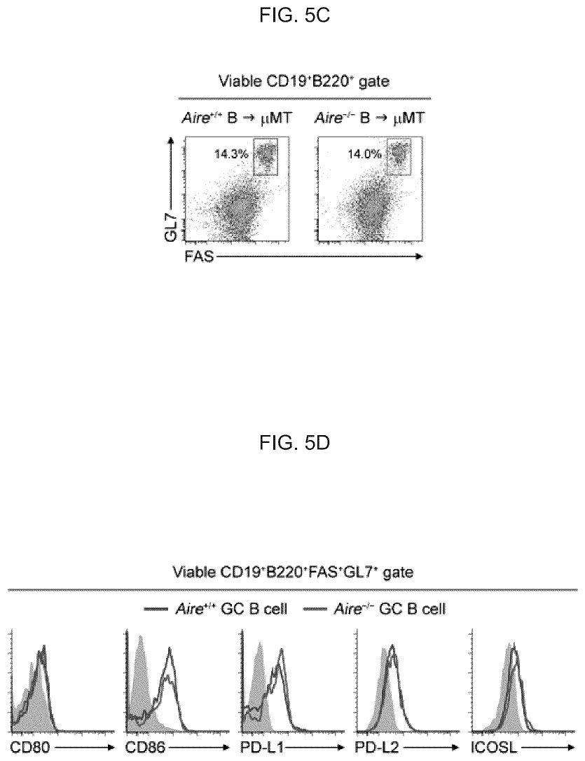

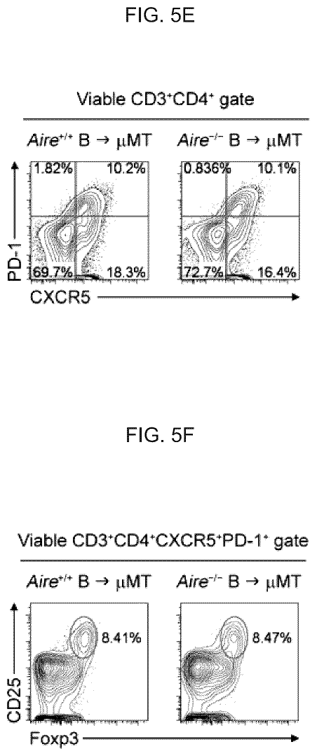

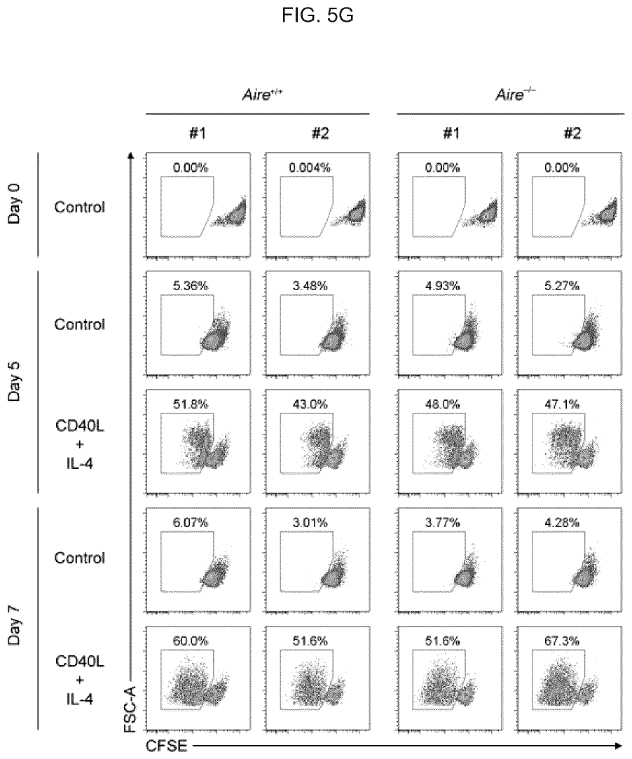

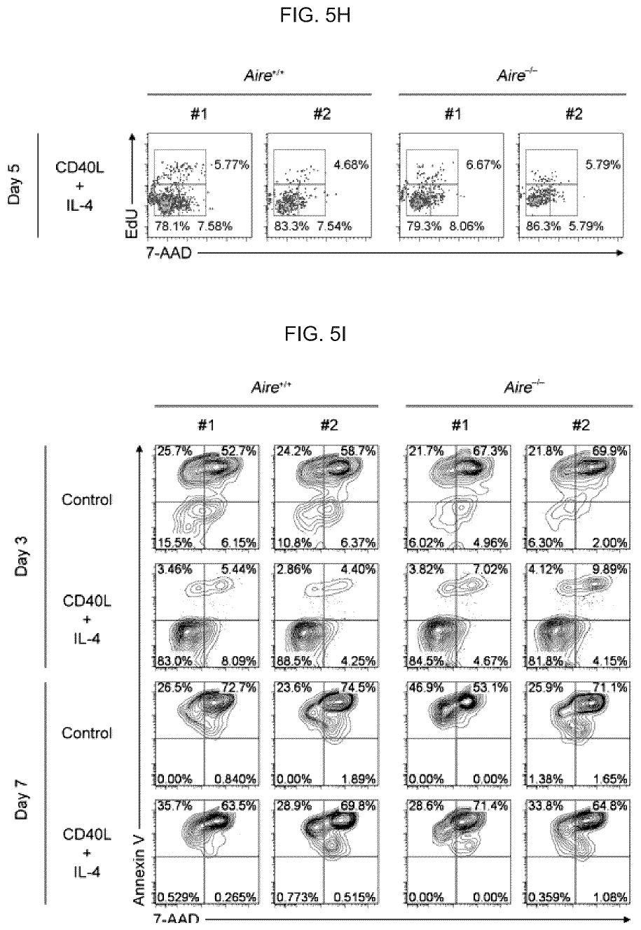

The B cell-deficient μMT recipient mice reconstituted with resting B cells from either Aire^{+/+} or Aire^{-/-} naive donor mice (FIG. 5A) was employed to determine the B cell-intrinsic function of AIRE in antibody response. Before adoptive transfer, Aire^{+/+} and Aire^{-/-} donor B cells exhibited a similar phenotype (FIG. 5B). Following repeated systemic immunization with the T cell-dependent antigen NP₃₂-KLH, Aire^{+/+} and Aire^{-/-} donor B cells equally entered the splenic GC compartment (FIG. 5C) and showed similar expression of major co-stimulatory and co-inhibitory molecules (FIG. 5D), but NP-specific Aire^{-/-} donor B cells exhibited elevated CSR by harboring a much higher fraction of IgM⁻IgD⁻ cells than NP-specific Aire^{+/+} donor B cells (FIG. 6A), and underwent increased affinity maturation by producing IgG1, IgG2b and IgG3, but not IgM, of higher NP₄ to NP₂₉ binding ratios (FIG. 6B). Of note, μMT recipients of Aire^{+/+} and Aire^{-/-} B cells had a similar proportion of CXCR5⁺PD-1⁺follicular helper T (T^{FH}) cells (FIG. 5E) and Foxp3⁺CD25⁺ follicular regulatory T (T_{FR}) cells in the spleen (FIG. 5F). These results suggest that AIRE inhibits antibody CSR and SHM in a GC B cell-intrinsic manner.

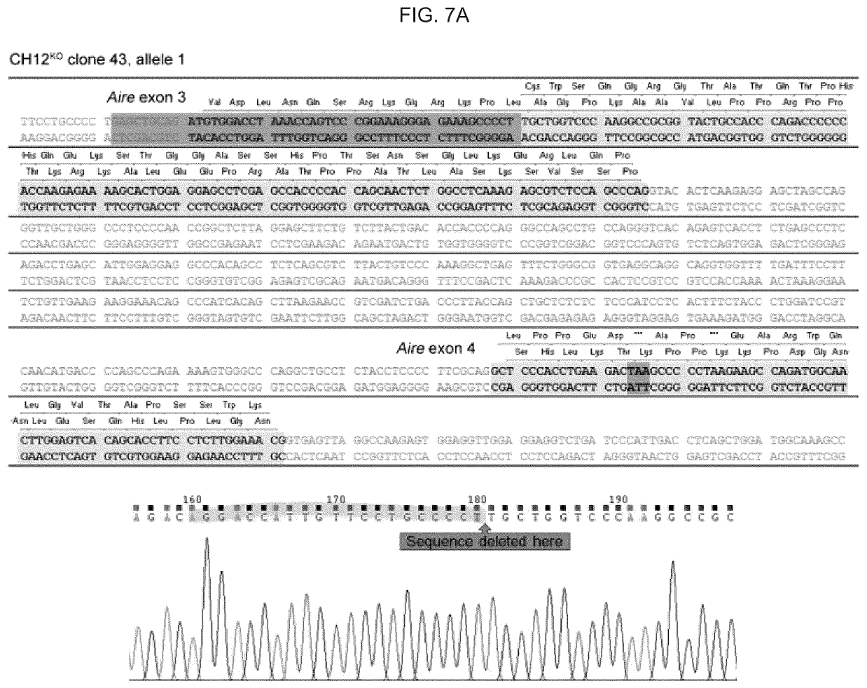

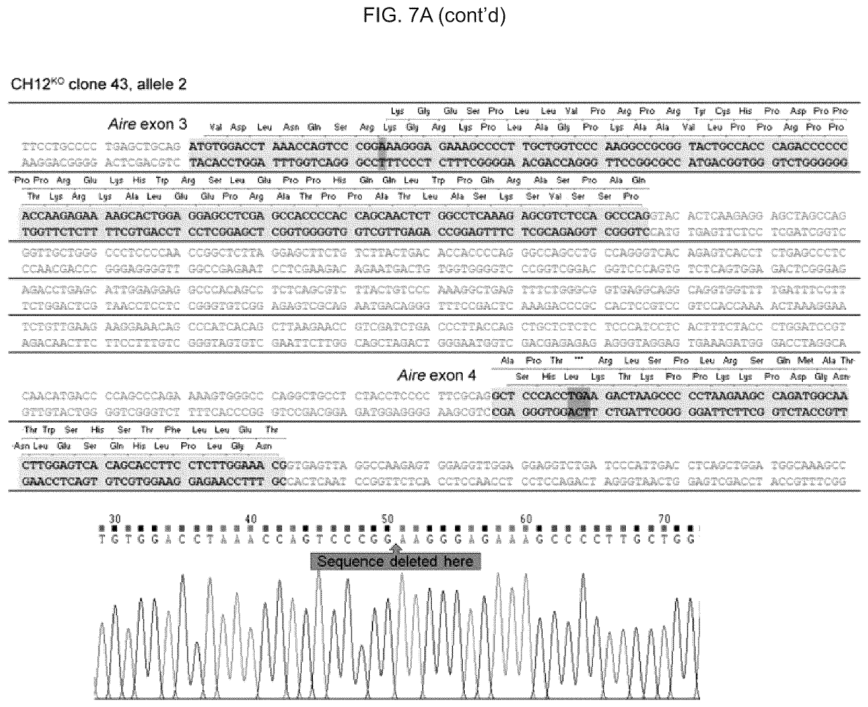

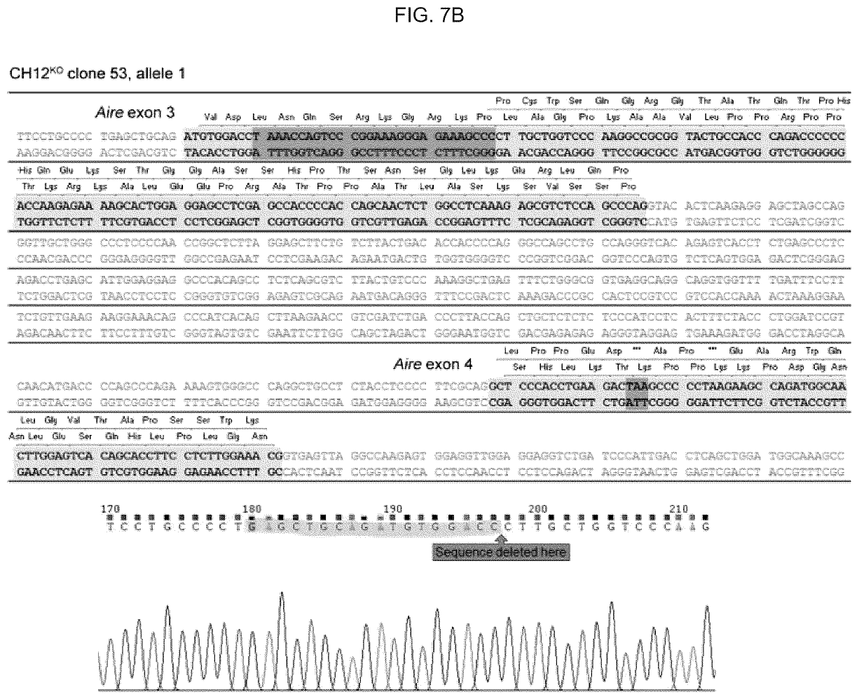

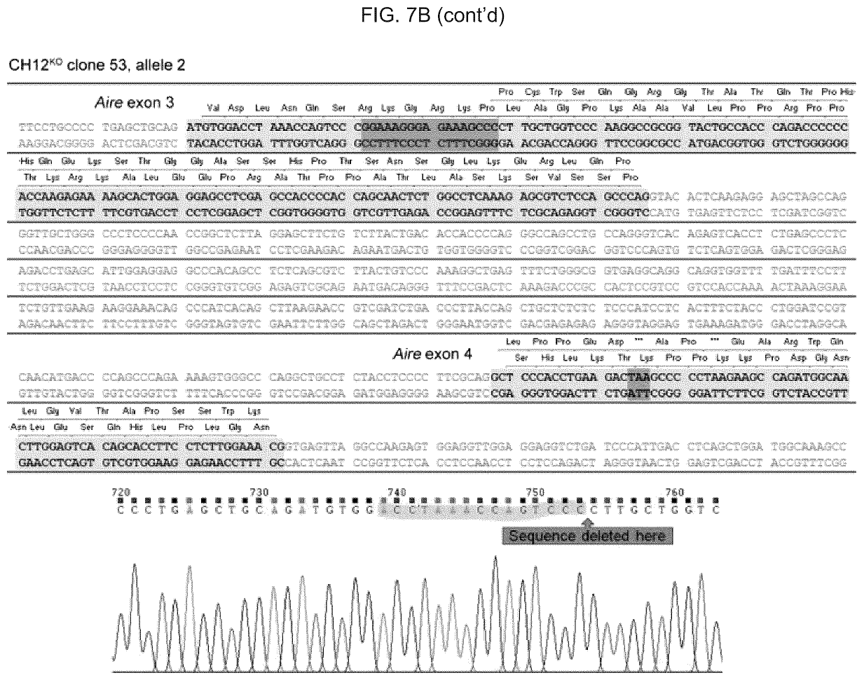

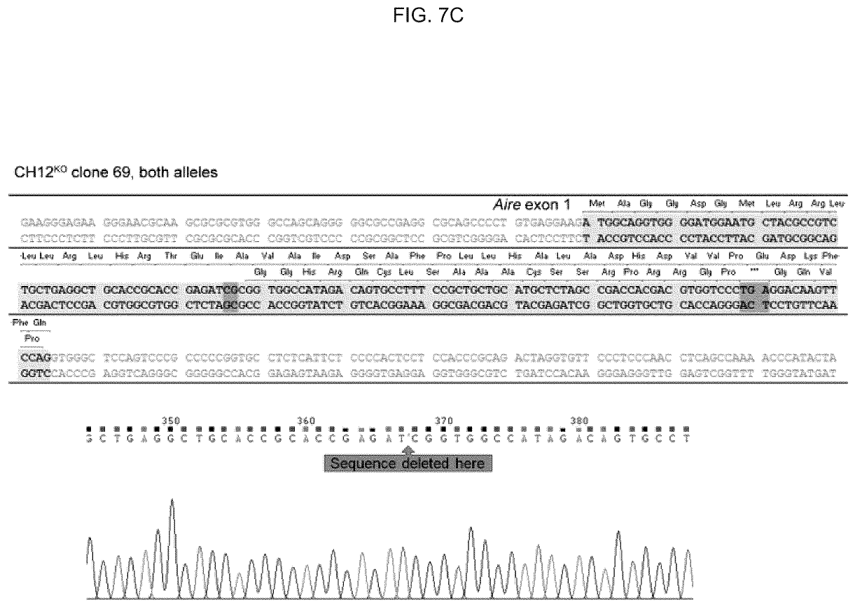

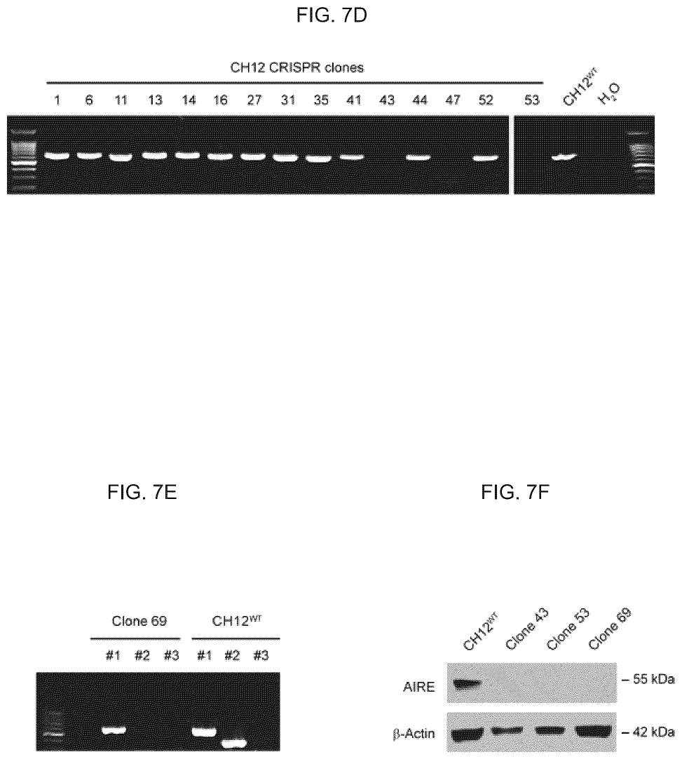

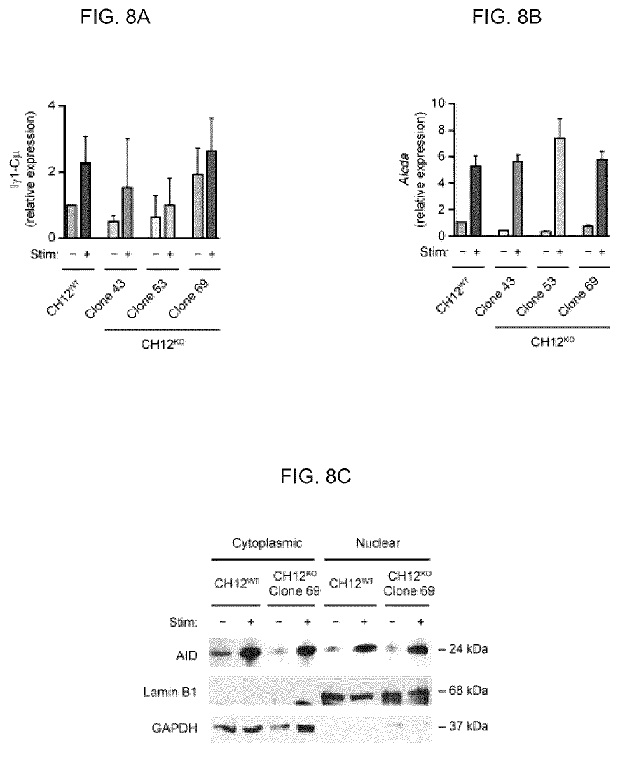

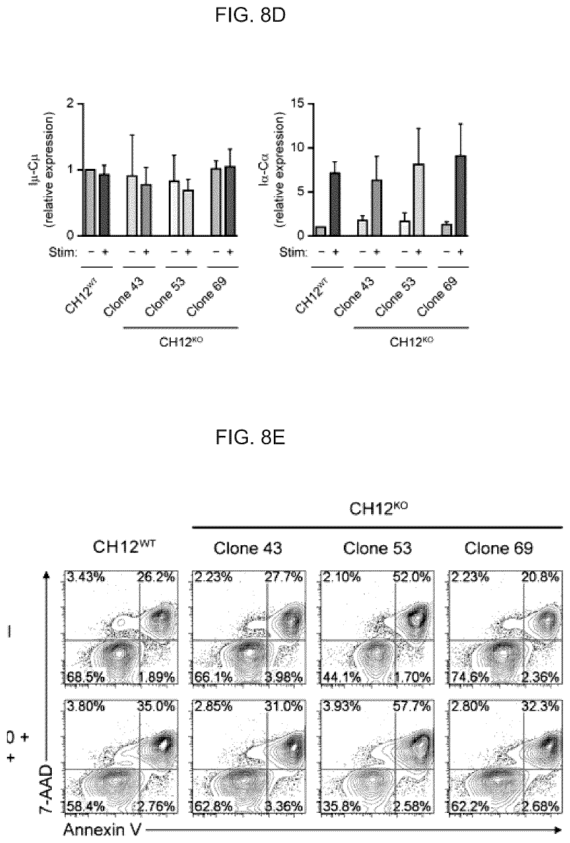

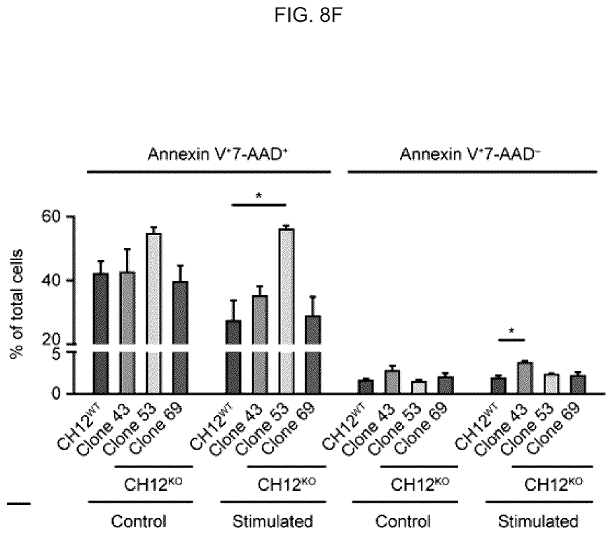

To verify the B cell-intrinsic inhibitory function of AIRE in CSR, splenic B cells of naïve Aire^{+/+} and Aire^{-/-} mice were compared for their ability to undergo CSR ex vivo. Aire^{-/-} B cells underwent increased CSR (FIG. 6C) and secreted more class-switched antibodies upon stimulation in culture (FIG. 6D). The enhanced CSR of and antibody production by Aire^{-/-} B cells were not caused by altered proliferation or apoptosis ex vivo (FIGS. 5G-51). Using CRISPR-mediated gene editing, the Aire gene was disrupted in CH12 cells, a mouse B cell line that class-switches from IgM to IgA upon stimulation with anti-CD40, TGF-β and IL-4 (Nakamura et al., International immunology 8, 193-201, (1996)), and identified 3 Aire^{-/-} CH12 clones which were frame-shifted in both Aire alleles (FIGS. 7A-7E), devoid of AIRE protein expression (FIG. 7F) and intact at CRISPR off-target sites (not shown). Upon stimulation, these Aire^{-/-} CH12 clones underwent elevated IgA CSR (FIGS. 6E, 6F) with concomitantly increased levels of the Iα-Cμ (FIG. 6G) but not ly1-Cμ circle transcript (FIG. 8A) compared to their parental Aire^{+/+} CH12 cells. Exaggerated IgA CSR in Aire^{-/-} CH12 cells was not a result of increased induction of the CSR-mediating enzyme activation-induced cytidine deaminase (AID) (FIGS. 8B, 8C) or germline transcription (FIG. 8D), nor a result of increased survival (FIGS. 8E, 8F). Remarkably, WT AIRE, but not a nuclear localization signal (NLS) deletion mutant AIRE (AIRE^{ΔNLS}), suppressed cytokine-induced CSR when re-introduced into Aire^{-/-} CH12 cells (FIG. 6H). These results reaffirm the B-cell intrinsic function of AIRE in inhibiting CSR.

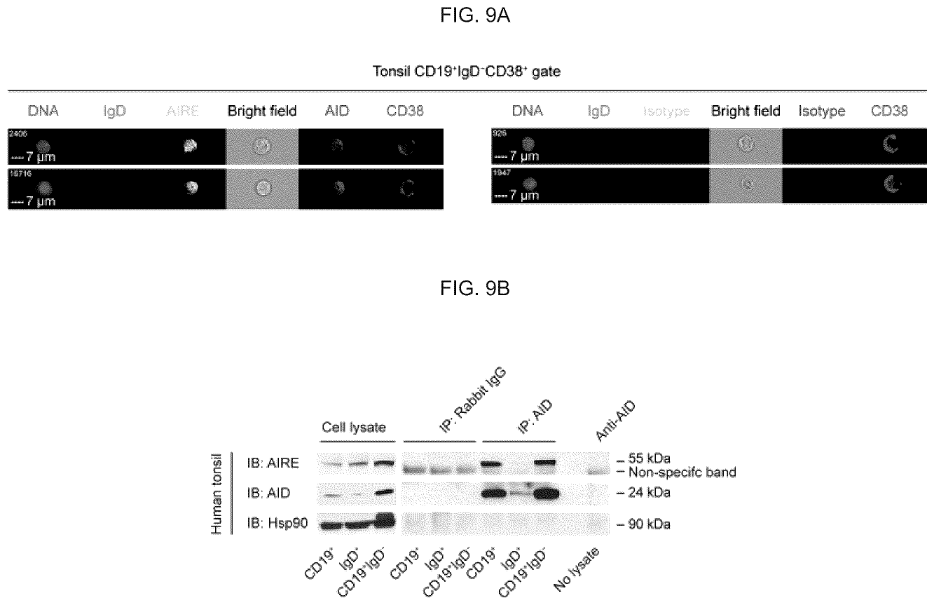

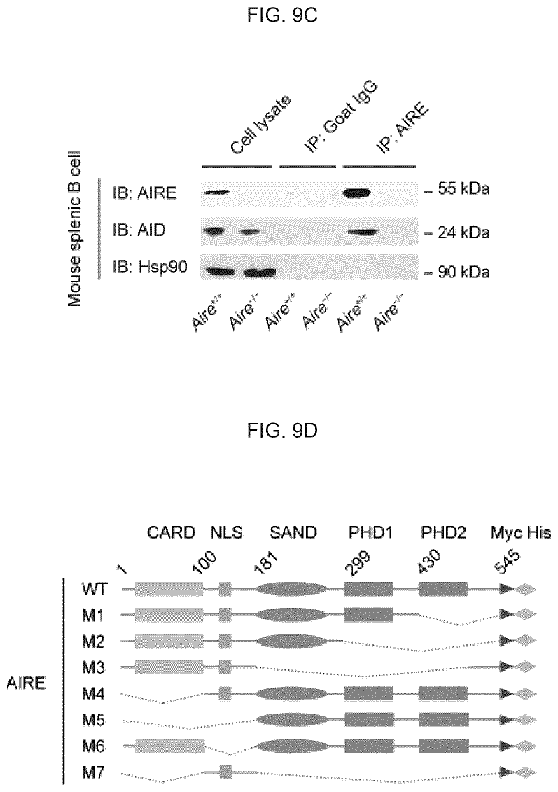

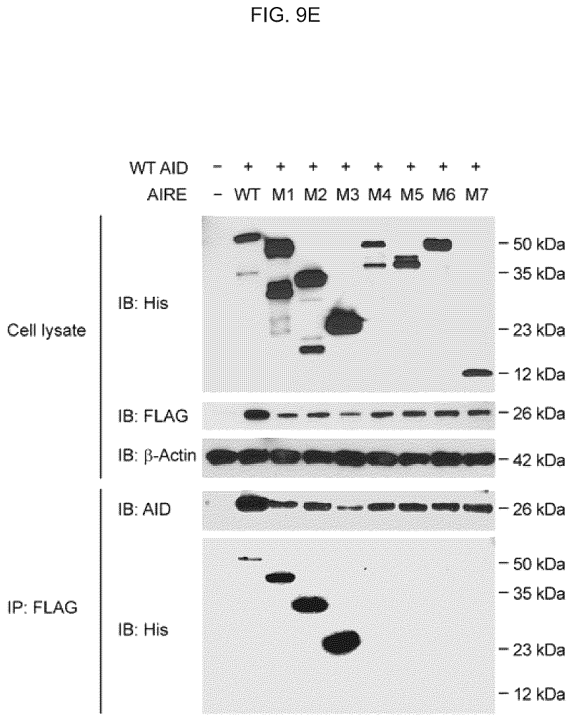

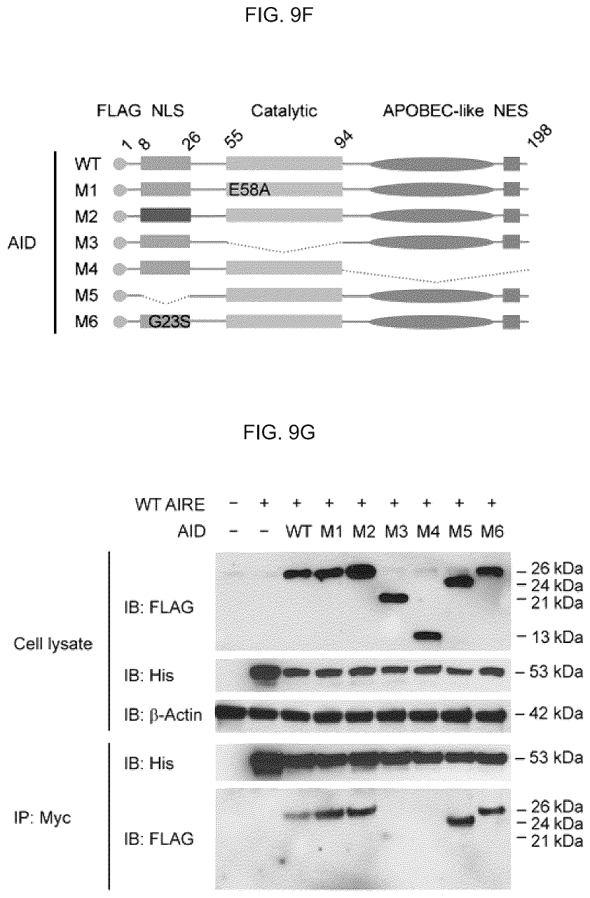

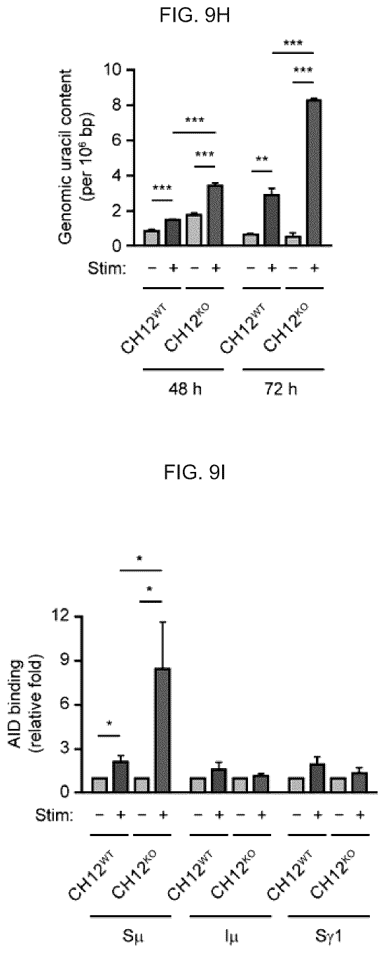

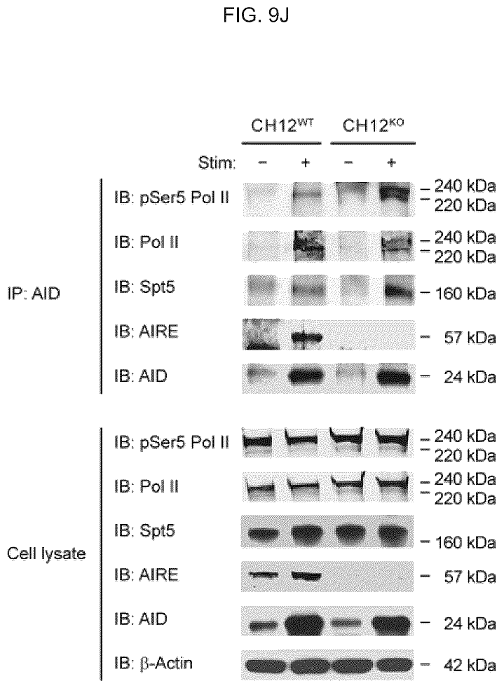

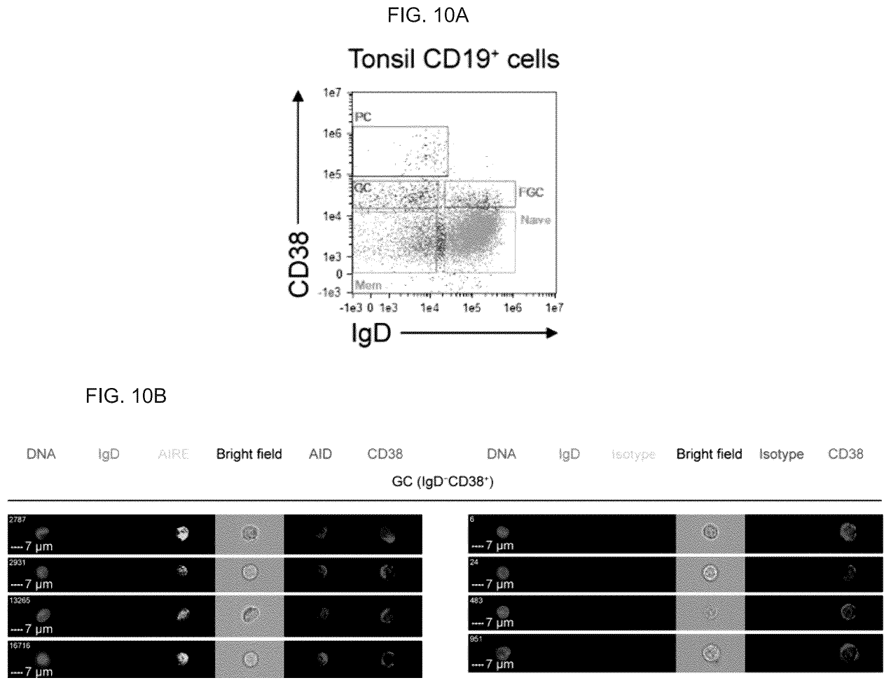

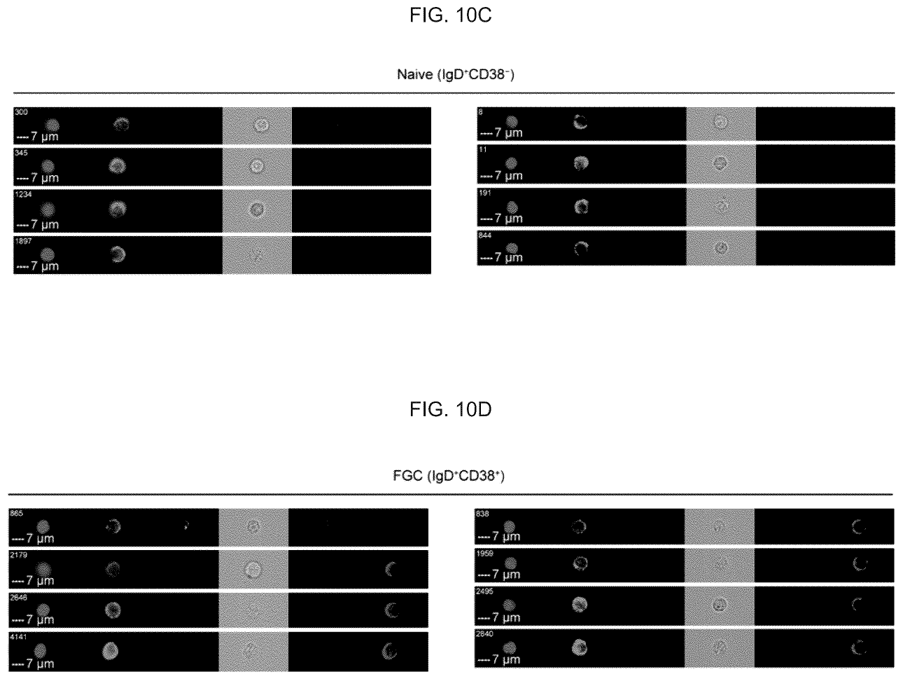

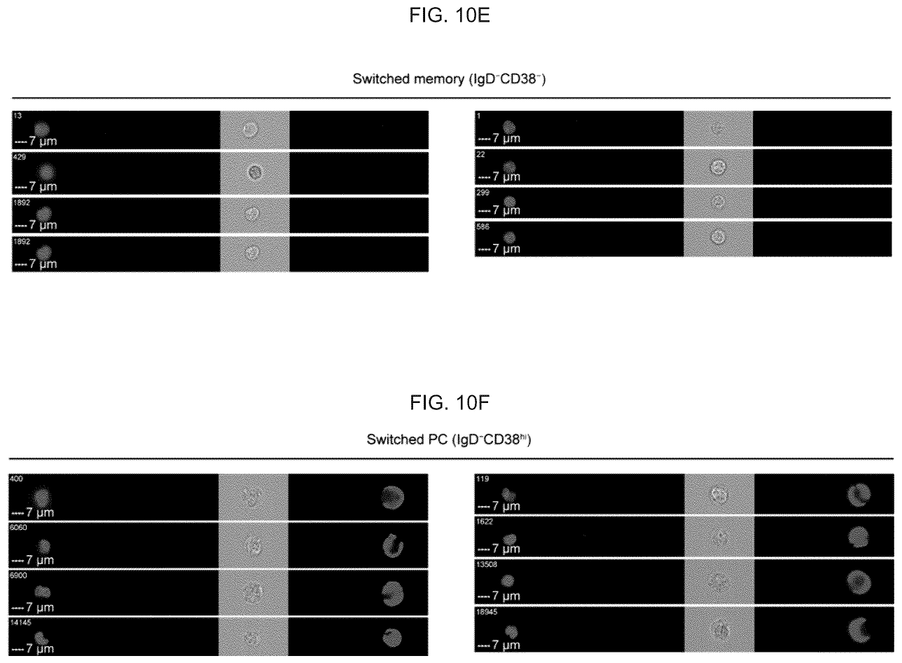

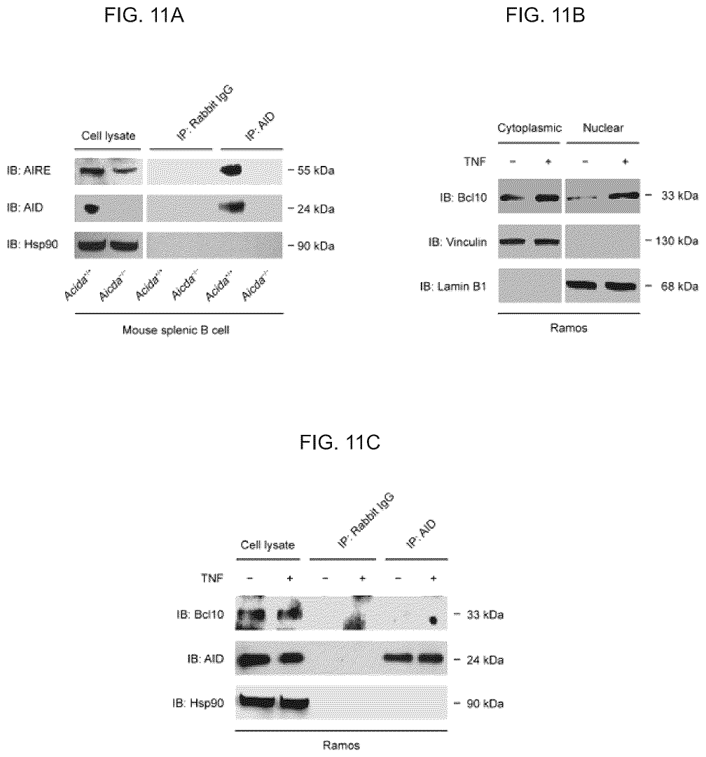

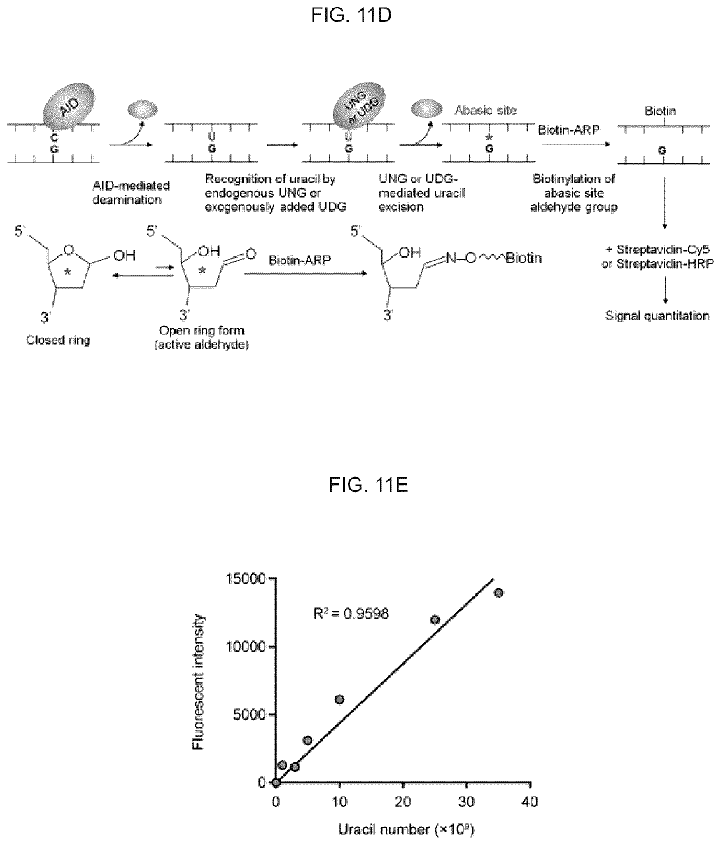

As AID is the obligatory enzyme that mediates CSR and SHM (Muramatsu et al., Cell 102, 553-563, (2000)), whether AIRE inhibits AID activity in B cells was examined. AIRE and AID co-localized in the nuclei of tonsillar IgD⁻CD38⁺ GC B cells (FIG. 9A, FIGS. 10A, 10B) but not in IgD⁺CD38⁻ naive B cells, IgD⁻CD38⁻ switched memory B cells or IgD⁻CD38^{hi} switched plasma cells (PCs), albeit a low level of nuclear AIRE and AID were detected in a small fraction of IgD⁺CD38⁺ founder GC (FGC) B cells (FIGS. 10C-10F). Using an AID antibody validated for immunoprecipitation (IP) and Chromatin IP (ChIP) (Vuong et al., Nature immunology 10, 420-426, (2009)), it was found that AIRE interacted with AID in human tonsillar CD19⁺ and CD19⁺IgD⁻ cell fractions (FIG. 9B). AIRE also co-immunoprecipitated with AID in splenic B cells of immunized WT but not Aire^{-/-} or Aicda^{-/-} mice (FIG. 9C, FIG. 11A). These data collectively demonstrate that AIRE interacts with AID in GC B cells in vivo and in B cell lines undergoing Ig diversification in vitro.

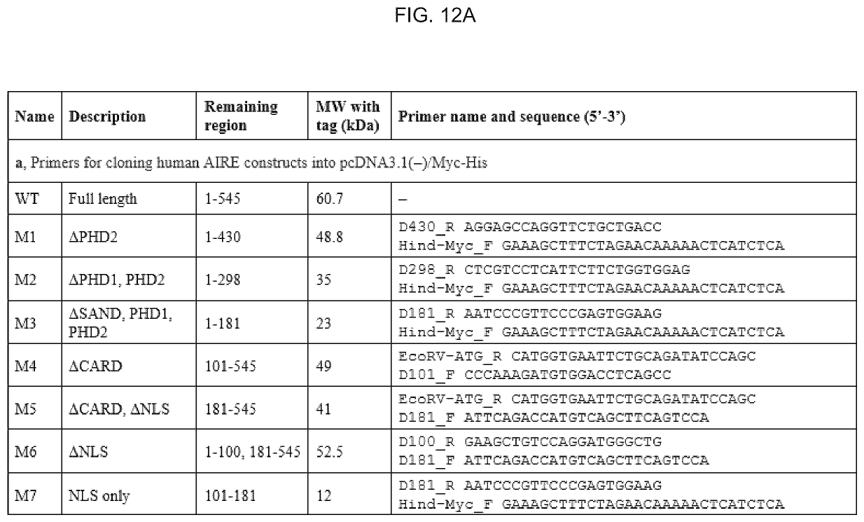

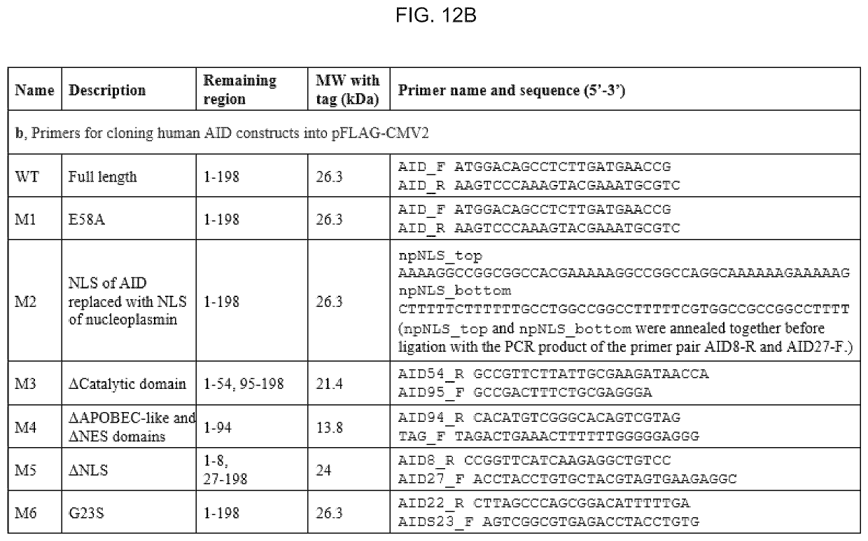

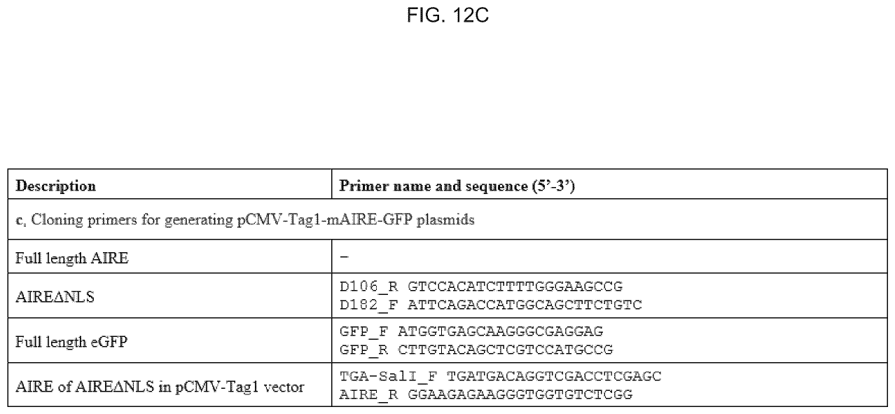

A series of deletion mutants of AIRE with C-terminal Myc and His tags were subsequently generated to characterize its interaction with AID (FIG. 9D, FIG. 12A). AIRE mutants missing the N-terminal caspase activation and recruitment domain (CARD) and/or nuclear localization signal (NLS) lost the ability to interact with AID (FIG. 9E), which demonstrates a requirement for the CARD and NLS of AIRE for interaction with AID and echoes the earlier result showing the dependence for NLS of AIRE in inhibiting CSR in CH12 cells (FIG. 6H). The CARD-dependent interaction with AID was specific to AIRE, as another CARD-containing protein, Bcl10, which undergoes TNF-induced nuclear translocation (Yeh et al., J Biol Chem 281, 167-175, (2006)) (FIG. 11B), did not interact with AID in the human Ramos B cell line which undergoes constitutive Ig diversification in culture (Sale & Neuberger, Immunity, 9, 859-869 (1998)) (FIG. 11C). Furthermore, using a series of deletion, domain replacement or point mutants of AID with an N-terminal FLAG tag (FIG. 9F, FIG. 12B), it was found that the interaction between AID and AIRE required both the catalytic and APOBEC-like domains of AID, although the catalytic activity of AID was not necessary, as the catalytically inactive AID^{E58A} mutant (Patenaude et al.,Nat Struct Mol Biol 16, 517-527, (2009)) still interacted with AIRE (FIG. 9G). The AID mutation G23S, which substantially abrogates SHM but not much CSR activity (Wei et al., Nature immunology 12, 264-270, (2011)), did not affect the interaction with AIRE (FIG. 9G).

A genomic uracil dot blot assay was then employed to directly test the effect of AIRE on the activity of AID (FIGS. 11D, 11E). Upon stimulation to undergo CSR, Aire^{-/-} CH12 cells harbored higher numbers of genomic uracil than Aire^{+/+} CH12 cells (FIG. 9H), reflecting an inhibitory role of AIRE in AID's deaminase activity. Maul et al., Nature immunology 12, 70-76, (2011). Considering that the function of AID requires its proper targeting to the Ig heavy chain (IgH) switch (S) regions at sites of Pol II stalling (Pavri et al., Cell 143, 122-133, (2010)), increased AID binding to the Sμbut not Iμor Sy1 region (FIG. 9I) and increased AID interaction with transcriptionally stalled (Ser5) Pol II and its associated factor Spt5 (Peterlin & Price,Molecular cell 23, 297-305, (2006)) in stimulated Aire^{-/-} CH12 cells compared to stimulated Aire^{+/+} CH12 cells (FIG. 9J) were found. These data are consistent with a function of AIRE in unleashing stalled Pol II by recruiting the positive transcription elongation factor b (P-TEFb) complex (Oven et al., Mol Cell Biol 27, 8815-8823, (2007)), and suggest that AIRE inhibits AID function by promoting Pol II escape from stalling and reducing AID targeting to its DNA substrate.

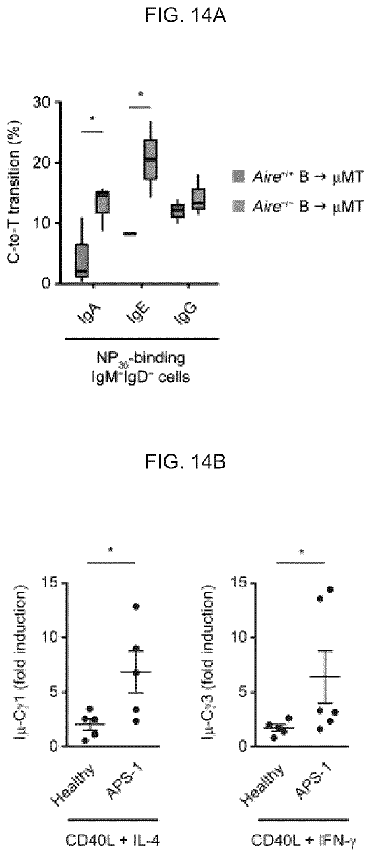

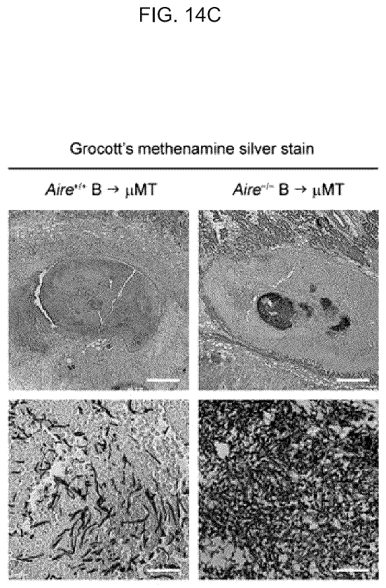

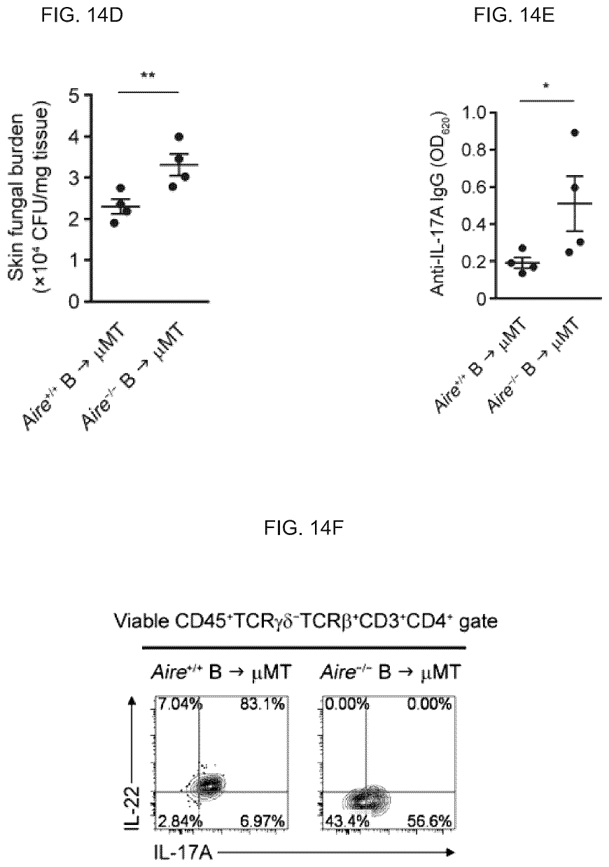

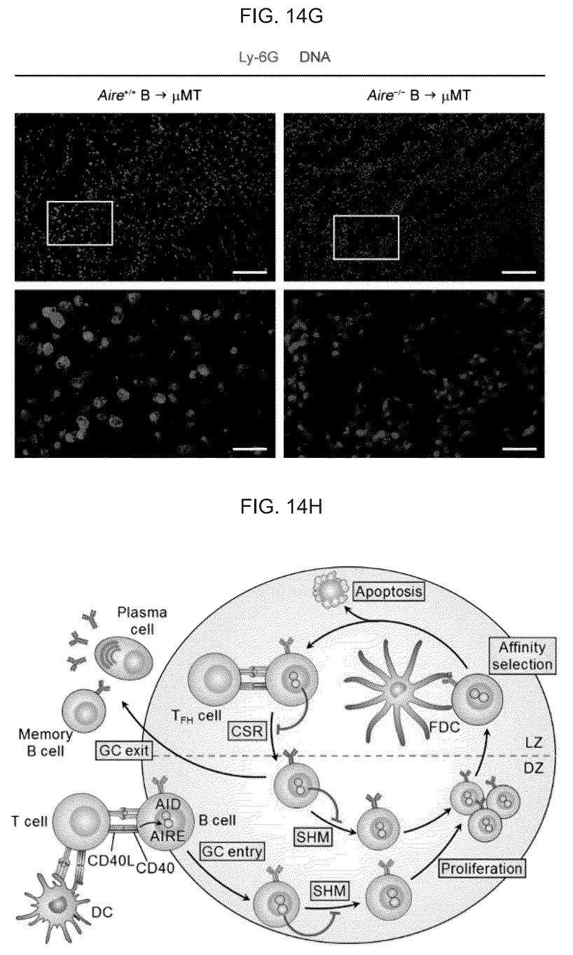

Given that the vast majority of APS-1 patients mysteriously develop chronic mucocutaneous candidiasis (CMC) as an early clinical symptom, which may result from the aberrant production of class-switched neutralizing autoantibodies against T_{H}17 cytokines that can impair anti-C. albicans immunity (Puel et al., The Journal of experimental medicine 207, 291-297, (2010); Kisand et al., The Journal of experimental medicine 207, 299-308, (2010); Meyer et al., Cell 166, 582-595, (2016)), the molecular and functional impact of B cell-intrinsic AIRE in humoral immunity and anti-Candida defense was sought. The IgH variable region (IgHV) SHM landscape of antigen-specific Aire^{+/+} and Aire^{-/-} donor B cells after repeated immunization of recipient μMT mice with NP₃₂-KLH (FIG. 13A) were first compared. NP-specific Aire^{-/-} B cells in the recipients' spleen that class-switched to IgG or IgE exhibited higher rates of IgHV SHMs in complementarity-determining region 2 (CDR2) and framework region 3 (FR3) than Aire^{+/+} donor B cells (FIG. 13B). There was also an increased frequency of C-to-T transitions in the SHMs in the IgHV coding sequences in NP-specific Aire^{-/-} donor B cells compared to Aire^{+/+} donor B cells (FIG. 14A), a signature associated with the action of AID in IgHV. Maul et al., Nature immunology 12, 70-76, (2011).

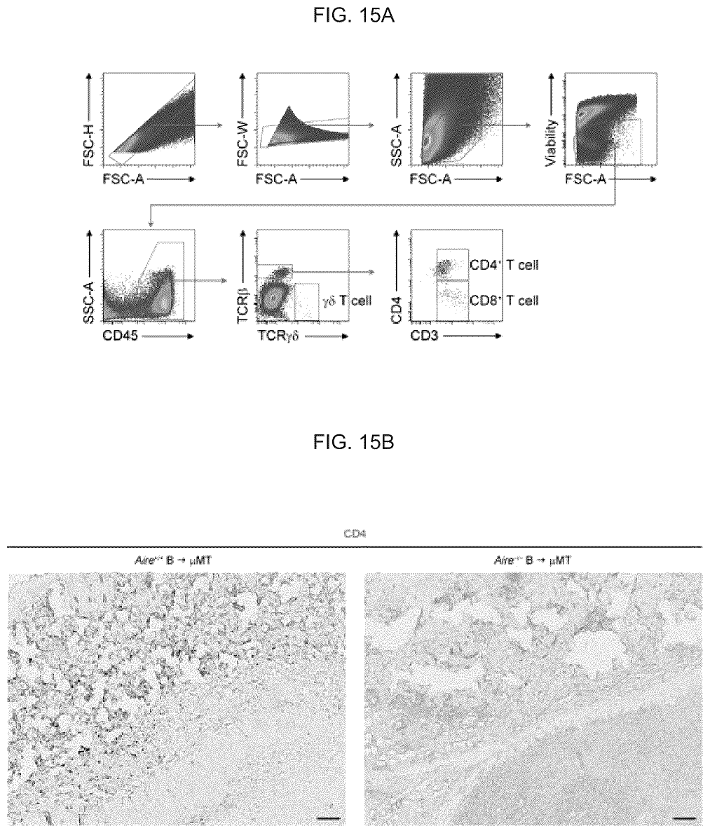

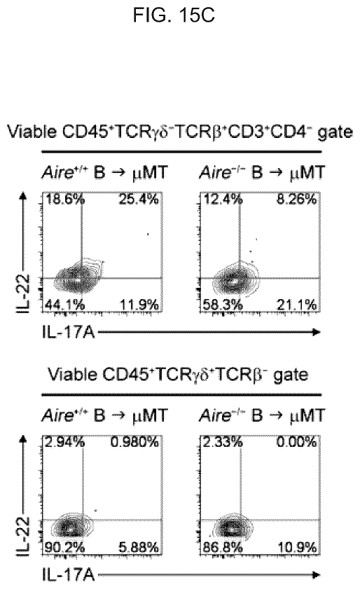

Furthermore, naïve B cells of APS-1 patients underwent elevated CSR than those of healthy donors upon stimulation ex vivo (FIG. 14B). It was then asked whether AIRE deficiency in peripheral B cells could promote APS-1-like CMC. After exposure to heat-killed C. albicans and subsequent cutaneous infection with live C. albicans pseudohyphae, μMT recipient mice of Aire^{-/-} B cells had heightened fungal burden in the skin 4 d after infection (FIGS. 14C, 14D) and concomitant elevation of autoantibodies to IL-17A, IL-17F and IL-22 in the sera as compared to μMT recipients of Aire^{+/+} B cells (FIG. 14E). μMT recipients of Aire^{-/-} B cells also had reduced IL-17A- and IL-22-producing CD4⁺ T cells (FIGS. 15A, 15B, FIG. 14F) but not yδ T cells (FIG. 15C), and diminished neutrophils infiltration (FIG. 14G) at the dermal infection site. Therefore, AIRE deficiency in peripheral B cells impairs cutaneous anti-Candida defense and promotes APS-1-like CMC by engendering humoral autoimmunity.

Collectively, this Example defines a crucial B cell-intrinsic AIRE-dependent GC checkpoint of peripheral antibody diversification which suppresses humoral autoimmunity that can arise from the GC reaction (Vinuesa et al., Nature Review. Immunology, 9, 845-857 (2009)) (FIG. 14H), and offers mechanistic insights into the production of high-affinity autoantibodies in many APS-1 patients. Puel et al., The Journal of experimental medicine 207, 291-297, (2010); Kisand et al., The Journal of experimental medicine 207, 299-308, (2010); Meyer et al., Cell 166, 582-595, (2016). These findings also highlight the emerging idea that peripheral tolerance mechanisms can be barriers to the generation of effective immunity, and controlled breaching of peripheral tolerance can permit neutralizing antibody responses that can be therapeutically beneficial. Meyer et al., Cell 166, 582-595, (2016); Schroeder et al., The Journal of experimental medicine, (2017); Rosenspire & Chen, Frontiers in immunology 6, 580, (2015). This has broad implications for new approaches of generating high-affinity neutralizing antibodies for therapeutic, diagnostic and research applications against, for example, infectious diseases and cancer.

Detailed Methods. Human subjects. Autoimmune polyglandular syndrome type 1 (APS-1) patients with loss-of-function mutations in the AIRE gene were enrolled in the study with an approved protocol of the Ethics Committee of Medicine of the Hospital District of Helsinki and Uusimaa (HUS), Finland. Hyper-IgM syndrome type 3 (HIGM3) patients with loss-of-function mutations in the CD40 gene were enrolled in the study with an approved Institutional Review Board (IRB) protocol of the Icahn School of Medicine at Mount Sinai. Peripheral blood leukocytes of anonymous healthy donors were obtained from the Southeast Michigan branch of American Red Cross with a protocol approved by the IRB of Wayne State University (WSU) and the Detroit Medical Centre (DMC). Tonsil, thymus and spleen tissues were obtained after pediatric tonsillectomy, cardiac surgery and splenectomy, respectively, from the Children's Hospital of Michigan with an IRB protocol approved by WSU and DMC.

Human blood and tissue sample processing and cell isolation. Peripheral blood mononuclear cells (PBMCs) of APS-1 patients and healthy controls were purified using Ficoll-Paque Plus (GE Healthcare 17-1440-03). Live (7AAD⁻ or Ghost Violet 510⁻) naive B cells (CD19⁺IgD⁺CD27⁻) and class-switched memory B cells (CD19⁺IgD⁻CD27⁺) were sorted from the PBMCs to a purity of ≥99% on a FACSAria II sorter (BD). PBMCs of anonymous healthy donors were isolated using a Histopaque-1077 gradient (Sigma-Aldrich 10771) following the manufacturer's instruction. Red blood cells (RBCs) were lysed using an ammonium-chloride-potassium (ACK) lysing buffer (Thermo Fisher Scientific A1049201). IgD⁺ or CD19⁺ B cells were purified from PBMCs by magnetic-activated cell sorting (MACS) with a biotinylated goat F(ab′)₂ anti-human IgD antibody and anti-biotin magnetic microbeads (Miltenyi Biotec 130-090-485) as previously reported. Chen et al., Nature immunology 10, 889-898, (2009).

The purity of the IgD⁺ B cells ranged from 92% to 99% as determined by flow cytometry with CD19 staining. CD19⁺ B cells were similarly separated from PBMCs using a biotinylated mouse anti-human CD19 (clone HIB19) antibody, with purity ranging from 94% to 98% as determined by flow cytometry using a different clone (SJ25C1) of CD19 antibody. Human tonsil and spleen tissues were minced into small pieces, meshed through 100 μm cell strainers, and pelleted at 600g for 5 min at 4° C. Spleen cells were treated with an ACK buffer to remove erythrocytes and filtered through 40 μm cell strainers. Tonsil and spleen cells were then washed with phosphate-buffered saline (PBS). Thymic cell suspensions were obtained by mincing human thymus tissues into small pieces and mechanically removing thymocytes followed by 2 rounds of digestion with 0.2% (w/v) Collagenase II (Worthington Biochemical LS004177) and 0.1 mg/ml DNase I (Roche 11284932001) in Hank's Balanced Salt Solution (HBSS) for 45 min at 3720 C. with shaking. The digested samples were filtered through 70 μm cell strainers and washed with PBS.

Mice. C57BL/6J mice (Jackson stock number 000664), Aire^{+/-} (B6.129S2-Aire^{tm1.1Doi}/J, Jackson stock number 004743) and μMT (B6.129S2-Ighm^{tm1Cgn}/J, Jackson stock number 002288) were purchased from the Jackson Laboratory. Aire^{Adig} mice in C57BL/6 background were previously reported. Gardner et al., Science, 321, 843-847 (2008). Aicda^{-/-} mice¹ were generously provided by Dr. Tasuku Honjo (Kyoto University, Japan). These mice were maintained in the same room at the specific pathogen-free (SPF) facility of the Division of Laboratory Animal Resources (DLAR) at Wayne State University. Aire^{-/-} mice were generated by mating Aire^{+/-} mice, and age- and sex-matched Aire^{+/+} littermates were used as controls for ex vivo and in vivo experiments. All breeding and experimental protocols were approved by Wayne State University Institutional Animal Care and Use Committee (IACUC).

Mouse blood and tissue cell isolation. Blood, spleen, inguinal lymph nodes, mesenteric lymph node and Peyer's patches were collected from euthanized mice. Adjacent fat and other tissues were removed before single cells suspensions were prepared, filtered through 100 μm cell strainer. RBCs from blood were removed by centrifugation on Histopaque 1077, and those in spleens were lysed using an ACK buffer. The cells were washed in PBS and counted before cell sorting, flow cytometry or purification by MACS. Resting B cells were isolated from the spleens of age- and sex-matched Aire^{+/+} or Aire^{-/-} littermates by MACS using a B Cell Isolate Kit (Miltenyi Biotec 130-090-862). The purity of the isolated B cells ranged from 97-99.6% as determined by flow cytometry based on CD19 and B220 staining.

Mouse immunization. 2.5×10⁷ purified Aire^{+/+} or Aire^{-/-} B cells were introduced via the tail vein into each recipient μMT littermate mouse. One day after the adoptive transfer, each recipient was intraperitoneally (i.p.) immunized with 1 dose of 100 μg NP₃₂-KLH (Biosearch Technologies N-5060) in Complete Freund's Adjuvant (Thermo Fisher Scientific 77140) and 4 doses of 100 μg NP₃₂-KLH in Incomplete Freund's Adjuvant (Thermo Fisher Scientific 77145) once every week. Four days after the last immunization, mice were sacrificed and blood and spleens were collected for ELISA, flow cytometry or cell sorting. In some experiments, mice were immunized with 200 μl of 2% sheep red blood cells in sterile PBS for 3 times, with each dose being 1 week apart.

Candida albicans culture. A single colony of C. albicans (ATCC MYA-2876) was cultured in YPD broth (BD 242820) at 30° C. for 16 h with shaking at 220 rpm. C. albicans existed in blastospore form after the 16 h culture. The concentration of the culture was quantitated using a haemocytometer. The culture was diluted 1:10 with fresh YPD broth containing 10% (v/v) heat-inactivated FBS (Thermo Fischer Scientific 26140079) and grown at 37° C. for 3 h with shaking at 220 rpm. An aliquot of the culture was removed and examined under the microscope to ensure that 95% of blastospores switched to the virulent pseudohyphal form. The culture was pelleted by centrifugation at 4,000 rpm for 10 minutes, washed with PBS twice and resuspended in PBS at the concentration of 5×10⁶ CFU per 50 μl based on the quantitation of the culture 3 h ago. The pseudohyphae samples were used for either intradermal infection of mice or the preparation of heat-killed samples by treatment at 95° C. for 2 h followed by 3 rounds of sonication on ice at 30% maximum power for 5 seconds per round using a sonifier (Thermo Fisher Scientific Q500).

Cutaneous C. albicans infection of mice. 5×10⁷ purified Aire^{+/+} or Aire^{-/-} B cells were introduced via the tail vein into each recipient μMT mouse littermate. Starting from the day of adoptive transfer, 5 doses each of 10⁶ CFU heat-killed C. albicans pseudohyphae were given intraperitoneally to each recipient mouse every 4 d. Four days after the last injection, mice were infected with 5×10⁶ CFU live C. albicans pseudohyphae in 50 μl PBS per spot at the deep dermis of the shaved dorsal region. Conti et al., Curr Protoc Immunol 105, 19 16 11-19 16 17, (2014). The actual dose of infection was determined by immediately plating serial dilutions of the inoculum on YPD agar in triplicate, incubating the plates at 28° C. for 24 h and colony enumeration. The inoculum size per spot ranged between 3.8-12.3×10⁶ CFU in various experiments. Four days after the infection, blood was obtained after sacrificing the mice. The entire dermal injection site was excised for histological evaluation of fungal burden by Grocott's methenamine silver (GMS) stain or by plating, or for determination of effector T cell response by flow cytometry. For GMS stain, the tissues were immediately fixed in 10% formalin overnight and embedded in paraffin before sectioning. For plating, each tissue was weighed, minced, grounded thoroughly and resuspended in sterile PBS. Serial dilutions of the suspensions were plated on YPD agar in triplicate and incubated at 28° C. for 24 h before colony enumeration. The fungal load was calculated as CFU per mg of tissue. For flow cytometry, the tissues were washed in FBS-free RPMI-1640 twice, minced and digested in FBS-free RPMI-1640 containing 0.7 mg/ml collagenase II (Worthington LS004177), 2 mM EDTA and 25 mM HEPES at 37° C. for 1 h. The digested samples were passed through a 70 μm cell strainer, washed twice with RPMI-1640 containing 10% FBS, 2 mg/ml glutamine, 100 U/ml penicillin, 100 μg/ml streptomycin and 25 μg/ml amphotericin B. The samples were then cultured in this medium further supplemented with 500 ng/ml PMA, 500 ng/ml ionomycin and 1 μg/ml GolgiPlug (BD 555029) at 37° C. for 5 h before being harvested for flow cytometric analysis.

Culture and stimulation or primary B cells. Peripheral blood IgD⁺ or CD19⁺ B cells of healthy donors, CD19⁺IgD⁺CD27⁻ B cells of APS-1 patients or mouse splenic B cells were cultured in RPMI-1640 medium (Sigma-Aldrich R8578) supplemented with 2 mM L-glutamine, 2 mg/ml NaHCO₃, 100U/ml penicillin, 100 μg/ml streptomycin, 0.25 μg/ml amphotericin B and 10% FBS (Thermo Fisher Scientific 26140-079 or Sigma-Aldrich F4135). Peripheral blood IgD⁺ or CD19⁺ B cells of healthy donors were stimulated with 500 ng/ml soluble CD40L (sCD40L) (Peprotech 310-02) and 100 ng/ml IL-4 (Peprotech 200-21) or 100 ng/ml IL-21 (Peprotech 200-04). Peripheral blood IgD⁺CD27⁻ naive B cells of healthy subjects or APS-1 patients were stimulated with 500 ng/ml sCD40L and 100 ng/ml IL-4 or 100 ng/ml IFN-y (Peprotech 300-02). Purified mouse splenic B cells were stimulated with 500 ng/ml sCD40L (Peprotech 315-15) with or without 100 ng/ml IL-4 (Peprotech 214-14), 100 ng/ml IL-21 (Peprotech 210-21) or 25 μM CAPE (Cayman Chemical 70750). In some experiments, sCD40L was replaced with 5 μg/ml anti-CD40. To determine cell proliferation, the cells were labelled with carboxyfluorescein succinimidyl ester (CFSE) (Biolegend 422701) according to the manufacture's protocol prior to culture. Alternatively, 10 μM 5-ethynyl-2′-deoxyuridine (EdU) was added to the culture medium for 6 hours before EdU incorporation was determined by flow cytometry using a Click-iT EdU Flow Cytometry Assay Kit (Thermo Fisher Scientific C10418) according to the manufacturer's protocol.

Culture and stimulation of B cell lines. The human IgM⁺IgD⁺ 2E2 B cell line (He et al., Journal of immunology 173, 4479-4491, (2004)) and Ramos B cell line (ATCC CRL-1596) were cultured in the above RPMI-1640 medium. 2E2 cells were stimulated with 500 ng/ml sCD40L with 100 ng/ml IL-21. WT CH12 cells (from Dr. Tasuku Honjo, Kyoto University, Japan) and Aire^{-/-} CH12 cells were cultured in the above RPMI-1640 medium further supplemented with 5% (v/v) NCTC-109 (Sigma-Aldrich N1140) and 50 μM β-mercaptoethanol (Sigma-Aldrich M3148). To induce IgA switching, the cells were stimulated with 1 μg/ml anti-mouse CD40 (eBioscience 16-0402), 12.5 ng/ml IL-4 (R&D 404-ML) and 1 ng/ml TGF-β1 (R&D 7666-MB/CF) for 3 d. The human embryonic kidney cell/Burkitt's lymphoma fusion cell line HKB-11 (ATCC 12568) was cultured in DMEM/F12 (Sigma-Aldrich D8437) supplemented with 2 mM L-glutamine, 100 U/ml penicillin, 100 μg/ml streptomycin, 0.25 μg/ml amphotericin B and 10% FBS.

Generation and validation of Aire^{-/-} CH12 cells. Several clones of Aire^{-/-} CH12 cells were generated by targeting the Aire gene using the CRISPR/Cas9 system as described in Ran et al., Nat Protoc 8, 2281-2308, (2013). Single guide RNAs (sgRNA) targeting exon 1 or exon 3 of mouse Aire gene (GenBank NC_000076.6) were designed using the online tool at crispr.mit.edu. Sequences with the highest score for the respective region were selected to express sgRNAs, pairs of oligonucleotides were synthesized and cloned into pSpCas9(BB)-2A-Puro plasmid (Addgene 48139) as reported in Ran et al., Nat Protoc 8, 2281-2308, (2013). The sgRNA expression plasmid was then transfected into CH12 cells using electroporation (square wave pulse at 200 V for 30 ms) in serum-free RPMI-1640 with 5 mM glutathione in a 4-mm cuvette. 24 hours after transfection, cells were resuspended in 125 ng/ml puromycin for 48 hours. After a brief expansion in puromycin-free media, single cell clones from transfected cells were screened for loss of the sgRNA targeting site using PCR. Clones with deletions in both alleles were identified by PCR. To determine the exact genomic modifications in each clone, the sgRNA-targeting sites were amplified with primer pairs spanning the targeting sites, and PCR products were sequenced directly using the respective forward primer. In addition, PCR products from clones 43 and 53 were cloned into the pGEM-T Easy vector and sequenced with T7 primer. All three mutant clones used were confirmed to harbor frameshift mutations on both alleles, resulting in termination shortly after the frameshift site. The potential off-target sites in the mouse genome for each guide were identified by the same online tool (crispr.mit.edu). Cas9 generally does not tolerate more than 3 mismatches. Hsu et al., Nat Biotechnol 31, 827-832, (2013).

All off-target sites in a potential gene-coding region with non-zero scores (up to 4 mismatches) were verified by sequencing to be intact. The lack of AIRE protein expression in these clones was finally confirmed by Western Blot.

Plasmids. Full-length human AIRE cDNA sequence was cloned into pcDNA3.1(-) with tandem C-terminal Myc and 6-Histidine tag (Thermo Fisher Scientific V38520). Sequences coding various domains of AIRE were deleted using a Phusion Site-Directed Mutagenesis Kit (Thermo Fisher Scientific F541) using appropriate primers (FIG. 12A). Briefly, to delete a specific section of AIRE in the vector, a pair of outward primers was designed to amplify the remaining region together with the plasmid backbone. PCR product was then phosphorylated at 5′ end and ligated with Quick T4 ligase (New England Biolabs M2200L) to recircularize it. Human AID was obtained by cloning full-length AICDA into pFLAG-CMV2 vector with an N-terminal FLAG tag (Sigma-Aldrich E7033). Domain-specific deletion mutants and G23S and E58A point mutants of AID were generated using the Phusion Site-Directed Mutagenesis kit using appropriate primers (FIG. 12B). The full-length Egfp sequence from pcDNA3-eGFP (from Dr. Thilo Hagen, National University of Singapore) was then cloned in frame to the C-terminus of AIRE or AIREΔNLS using blunt end ligation of PCR-amplified fragments (FIG. 12C).

Transfection. 10⁶ seeded HKB-11 cells were cultured to 70-90% confluence and transfected with 4 μg plasmid DNA using Lipofectamine 3000 (Thermo Fisher Scientific L3000015) in Opti-MEM (Thermo Fisher Scientific 31985070) by following the manufacturer's instruction. 10⁶ CH12 cells were suspended in a 4-mm electroporation cuvette containing 600 μl sterile PBS. Electroporation was performed using the Bio-Rad Gene Pulser Xcell system (voltage=550 V, capacitance=50 μF, resistance=∞, time constant=1.2 ms). The electroporated cells were transferred to 10 cm culture dishes, subsequently divided equally into 2 dishes containing the CH12 cell culture media supplemented with 250 μg/ml Geneticin, with one dish left unstimulated and the other stimulated with 1 μg/ml anti-CD40, 1 ng/ml TGF-β1 and 12.5 ng/ml IL-4 for 4 d.

Immunoprecipitation. Cultured cells were harvested, washed with cold PBS twice and lysed with a CelLytic™ M buffer (Sigma-Aldrich C2978) containing 1× protein inhibitor cocktail (Sigma-Aldrich P8340) and 1× Halt phosphatase Inhibitor (Thermo Fisher Scientific 78426) for 60 minutes on ice. The lysates were centrifuged at 12,000 g for 15 minutes at 4° C. Protein concentration in the supernatants was determined by a BCA Protein Assay Kit (Thermo Fisher Scientific 23225). Equal amounts of lysate supernatants were used for immunoprecipitation with specific or isotype control antibodies using protein G magnetic beads (Cell Signaling 8740 or Thermo Fisher Scientific 88847) according to the manufacturers' instructions.

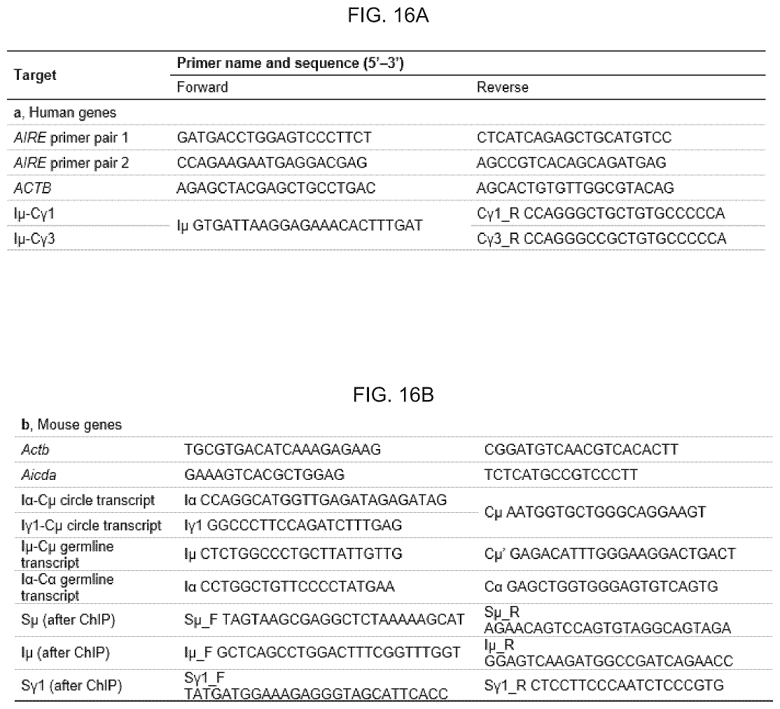

RNA extraction and quantitative real-time polymerase chain reaction. RNA was extracted from cells or tissues other than those from the APS-1 patients using TRIzol (Thermo Fisher Scientific 15596026). cDNA synthesis was performed using the Superscript III first strand synthesis system (Thermo Fisher Scientific 188080051) in a thermocycler (Bio-Rad T100). qRT-PCR was performed with PowerSYBR® Green Master Mix (Thermo Fisher Scientific 4367660) on a StepOnePlus instrument (Applied Biosystems) using pairs of sense and anti-sense primers targeting the genes of interest (FIGS. 16A, 16B). For APS-1 patients' peripheral blood IgD⁺CD27⁻ B cells, following stimulation, the cells were washed and stored in RNAlater® storage reagent (Thermo Fisher Scientific AM7020). Prior to RNA isolation, cells were pelleted and the RNAlater® was removed. RNA was isolated using the lysis and stop solutions in a Cells-to-C_{T} 1-step SYBR Green kit (Thermo Fisher Scientific A25601) and amplified using an iTaq Universal SYBR Green One-Step kit (Bio-Rad 172-5150) on a StepOnePlus instrument using pairs of sense and anti-sense primers targeting the genes of interest (FIG. 16A). The ACTB (Actb) gene was used as an internal control for normalization.

Chromatin immunoprecipitation and quantitative real-time PCR. ChIP was performed using a ChIP assay kit (EMD Millipore 17-295) based on the manufacturer's instructions with slight modifications. Following 3 d of stimulation of 10⁶ CH12 cells as described above, formaldehyde was added to the culture to the final concentration of 1% and incubated for 10 minutes at 37° C. to crosslink chromatin. The cells were pelleted, washed twice in PBS, resuspended in an SDS lysis buffer (1% SDS, 10 mM EDTA, 50 mM Tris, pH 8.1) for 10 minutes on ice. DNA was sheared by 3 rounds of sonication on ice at 30% maximum power for 3 seconds per round using a sonifier (Thermo Fisher Scientific Q500). After centrifugation at 13,000 rpm for 10 minutes, the supernatants were harvested, diluted 10-fold in a ChIP dilution buffer (0.01% SDS, 1.1% Triton X-100, 1.2 mM EDTA, 16.7 mM Tris-HCl, 167 mM NaCl, pH 8.1) containing protease inhibitors, and precleared with 50% protein A agarose/salmon sperm DNA slurry for 30 minutes at 4° C. with rotation. After setting aside an aliquot as input, an AID or control antibody was then added and incubated overnight at 4° C. with rotation, followed by the addition of 50% protein A agarose/salmon sperm DNA slurry for 1 h at 4° C. with rotation. The agarose was then pelleted and sequentially washed once with the low salt wash buffer, once with the high salt wash buffer, once with the LiCl wash buffer and twice with TE buffer, all of which were components of the kit. DNA in the bound chromatin was eluted from the beads using an elution buffer (1% SDS, 0.1 M NaHCO₃, pH 8.0), reverse-crosslinked from proteins by incubation at 65° C. for 4 h in the presence of 200 mM NaCl, cleaned by 20 μg/ml RNase A treatment for 30 minutes at 37° C. followed by 40 μg/ml proteinase K treatment for 1 h at 45° C., purified using phenol/chloroform extraction followed by ethanol precipitation with carrier glycogen according to the kit's manual and resuspended in TE buffer for quantitative real-time PCR analysis using PowerSYBR® Green Master Mix (Thermo Fisher Scientific 4367660) with the primers in FIG. 16B on a StepOnePlus instrument (Applied Biosystems). The fold enrichment of DNA was calculated using the ΔΔC_{T} method with control antibody-precipitated samples as an internal reference, and further compared among different CH12 cells and treatments.

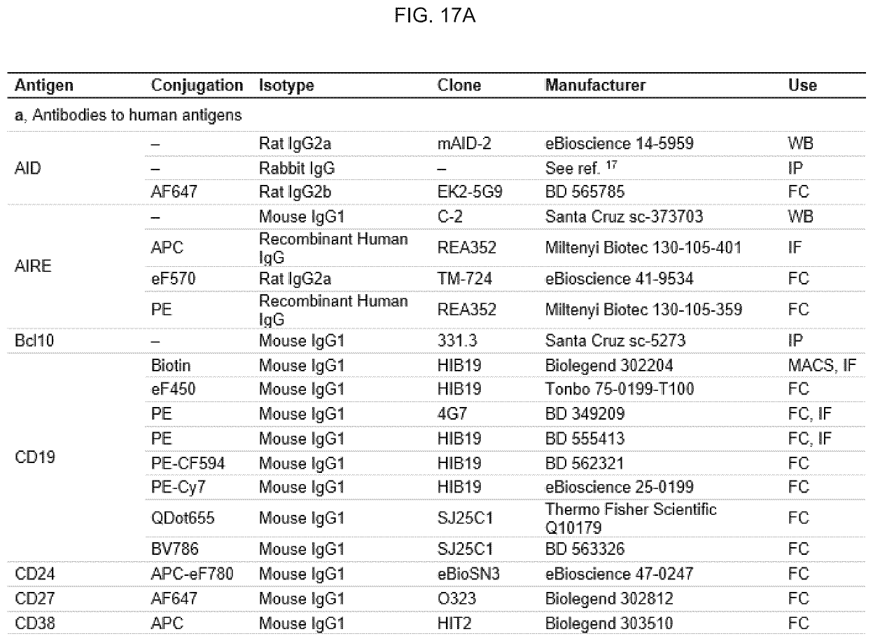

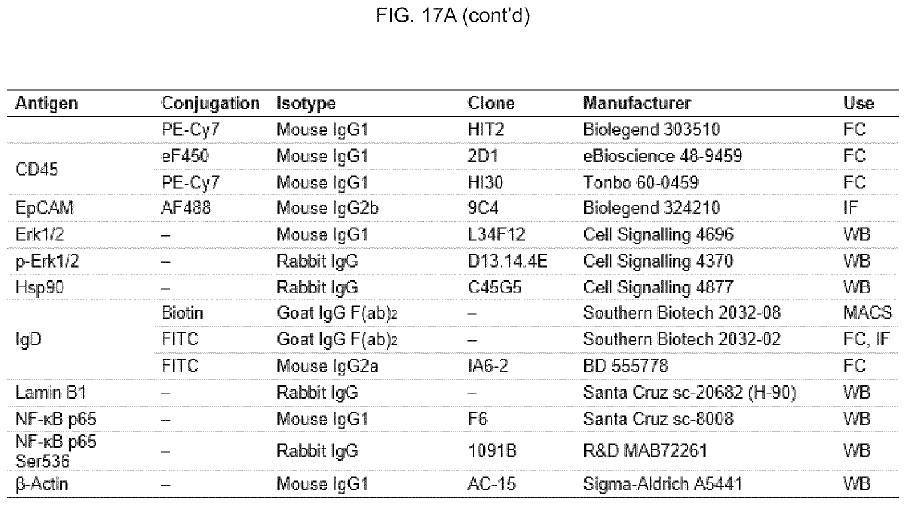

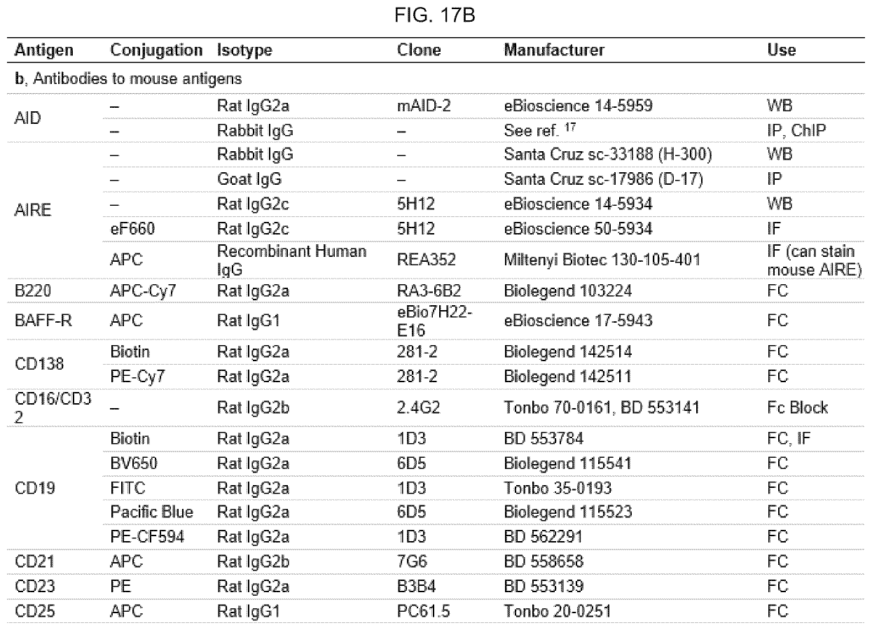

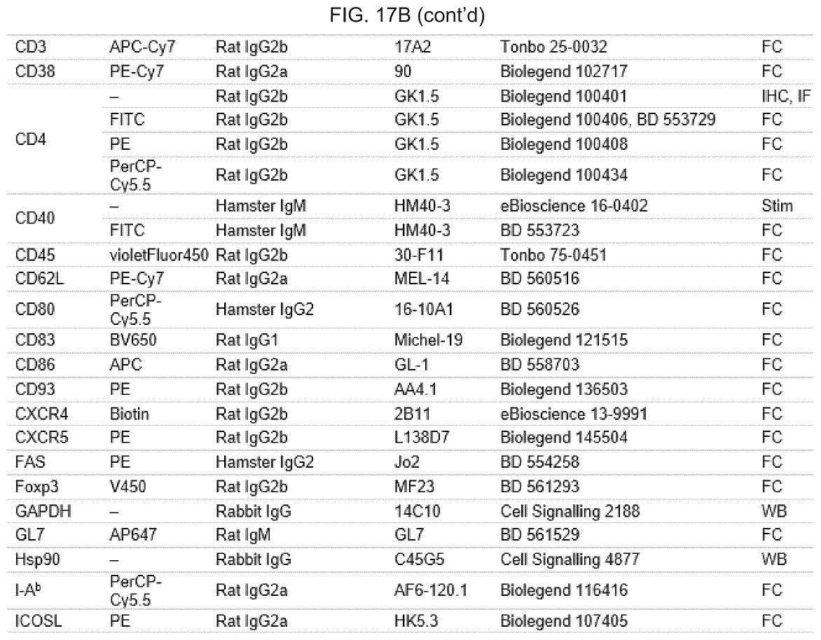

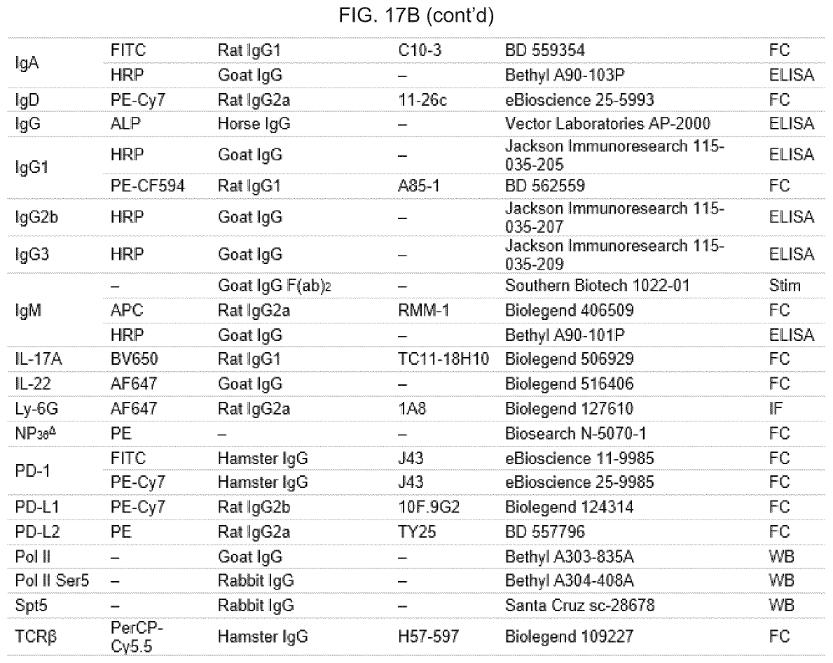

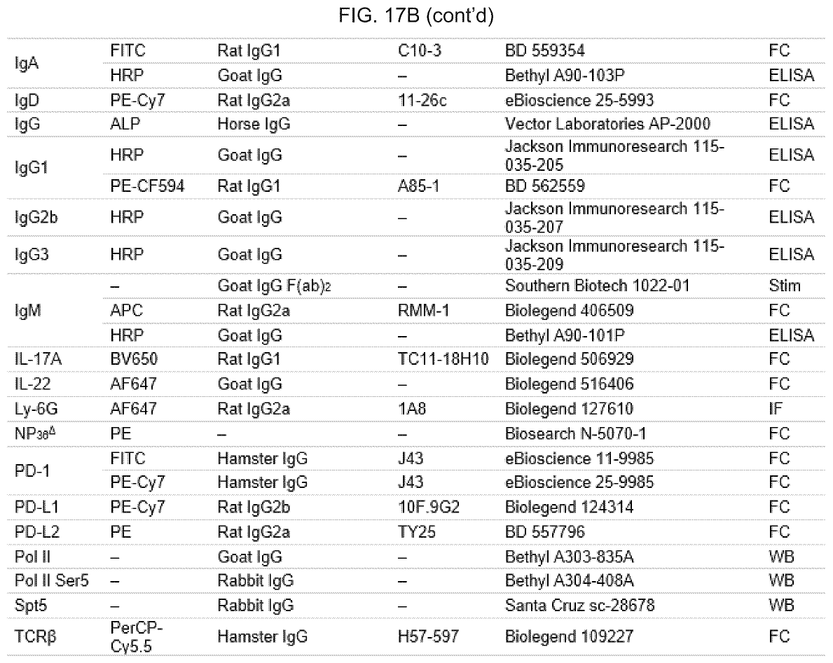

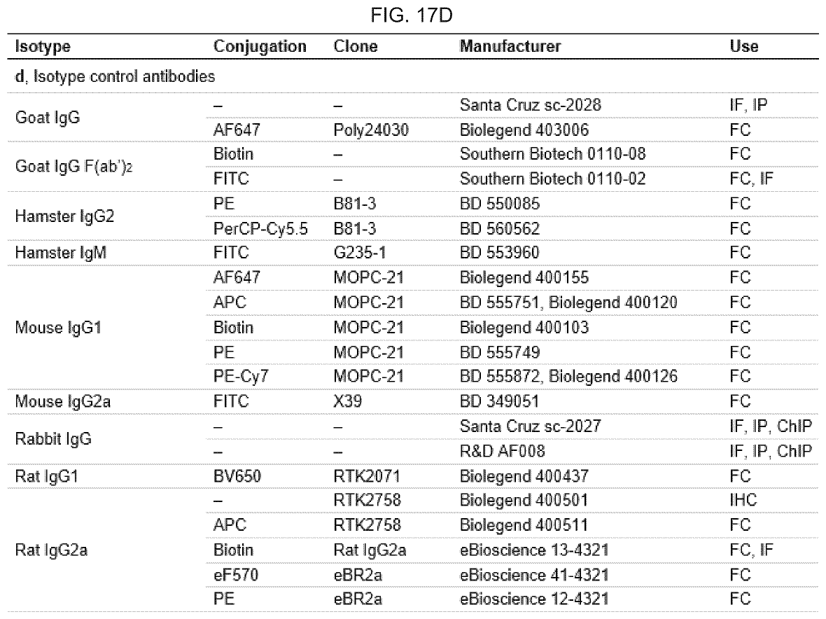

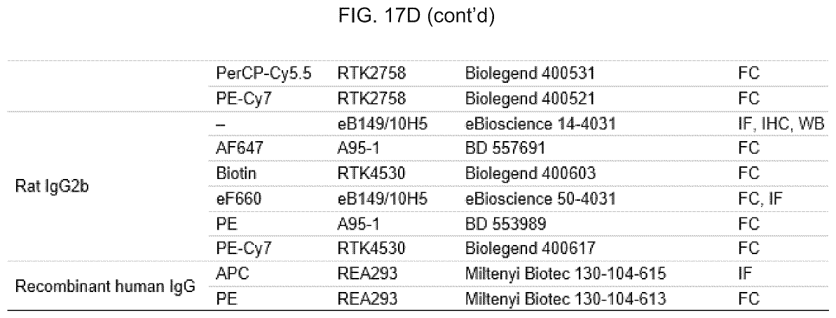

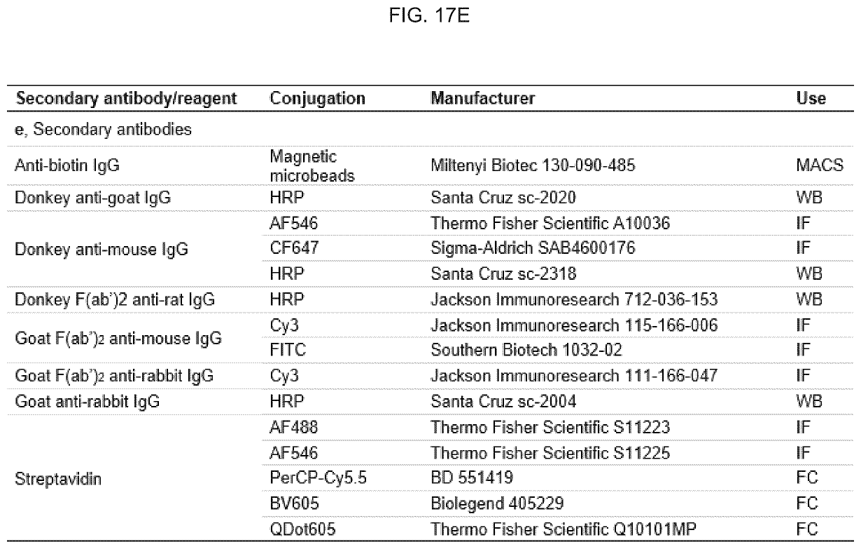

Protein extraction and Western Blot. Cells were pelleted and washed twice with ice-cold PBS, lysed with a pH 8.0 protein extraction buffer containing 20 mM Tris-HCl, 150 mM NaCl, 1% IGEPAL CA-630 (NP-40, Sigma-Aldrich 18896), 0.1% sodium dodecyl sulphate (SDS), 1 mM EDTA and protease and phosphatase inhibitor cocktail for 30 minutes on ice. Supernatants were collected after centrifugation, heated at 98° C. in SDS sample buffer with 4% β-mercaptoethanol for 5 minutes to denature proteins. Proteins were resolved in 4-20% Bis-Tris gels (GeneScript M42012) or 10% Tris-Glycine gels (Bio-Rad 4561034) and transferred to 0.2 μm polyvinylidene fluoride (PVDF) membranes (Bio-Rad 1620177). The membranes were blocked with 5% (w/v) non-fat milk in Tris-buffered saline with Tween-20 for 30 minutes to 1 h, incubated with primary antibodies (FIGS. 17A, 17B, 17D) overnight at 4° C. and subsequently with secondary antibodies conjugated to HRP (FIG. 17E). Signals were visualized with clarity western-blot ECL substrate (Bio-Rad 170-5061) and exposed on autoradiograph films.

Conventional flow cytometry. Cells were incubated with an Fc blocking reagent (Miltenyi Biotec 130-059-901 or Tonbo Biosciences 70-0161) and stained in PBS at 4° C. with antibodies to various cell surface antigens (FIGS. 17A, 17B, 17D). For staining of intracellular molecules, cells were subsequently fixed and permeabilized using a CytoFix/CytoPerm™ kit (BD 554722) or a Transcription Factor Buffer set (BD 562725). Isotype-matched control antibodies were used to define the baseline staining for the molecules of interest. Cells or beads stained with each fluorochrome were used to establish fluorescent compensation. 7-aminoactinomycin D (7-AAD, Tonbo Biosciences 13-6993-T500 or BD 559925) or Ghost Dye Violet 510 (Tonbo Biosciences 13-0870-T500) was used to identify and exclude non-viable cells from the analysis. Events were acquired on an LSR II or LSR Fortessa™ flow cytometer (BD) and analysed using FlowJo 7.6 (Tree Star).

Imaging flow cytometry. CD19⁺ B cells were purified from tonsillar cell suspensions by MACS with a biotinylated anti-CD19 antibody and anti-biotin microbeads (FIG. 17A). The cells were then incubated with an Fc blocking reagent and stained at 4° C. with antibodies to surface IgD and CD38, fixed and permeabilized, and stained for AID and AIRE or with isotype control antibodies, (FIGS. 17A, 17D). Nuclei were counter stained with 4′,6-diamidine-2′-phenylindole dihydrochloride (DAPI, Sigma-Aldrich D9542). Tonsillar cells stained with each fluorochrome were used to establish fluorescent compensation. Cells were imaged on an ImageStream® X Mark II imaging flow cytometer (Amnis®) and data were analysed using IDEAS 6.1 (Amnis).

Immunofluorescence analysis. Frozen human tissues were stored at −80° C. before 6-7 μm tissue sections were made using a cryostat (Leica CM1950). Sections were fixed with 4% paraformaldehyde, permeabilized in PBS containing 0.2% Triton X-100, blocked with PBS containing 1% BSA, 100 μg/ml human IgG and 10% serum from the source of the fluorochrome-conjugated antibodies, and stained with various combinations of primary antibodies against the molecules of interest (FIGS. 17A, 17B, 17D), followed by appropriate fluorochrome-conjugated secondary antibodies (FIG. 17E). Nuclei were visualized with DAPI. Following washing, slides were mounted using a FluoroSave™ reagent (EMD Millipore 345789) and imaged on a confocal microscope (Zeiss LSM 780 or Leica TCS SP5). Pseudocolor images were processed using Photoshop CS6 (Adobe).

ELISA. ELISA to determine NP-specific antibody affinity maturation was performed as previously described (Ballon et al., The Journal of clinical investigation 121, 1141-1153, (2011)) with minor modifications in the reagents. Briefly, each serum sample was titrated on both NP₂₉-BSA- and NP₄-BSA-coated microtiter plates. The ratio of binding to NP₄-BSA and NP₂₉-BSA is an indicator of relative Ig affinity maturation. Bound antibodies were detected using horseradish peroxidase (HRP)-conjugated goat-anti-mouse IgG1, IgG2b or IgG3 (FIG. 17B). The colorimetric reaction was terminated with the addition of an equal volume of 1 M H₂SO₄ and quantitated on a microplate reader (BioTek Epoch) at 450 nm. ELISA to determine IgG1 and IgA secretion by ex vivo stimulated mouse B cells was performed using a mouse IgG1 or IgA quantitation set (Bethyl E90-105 or E90-103). Anti-IL-17A, IL-17F and IL-22 autoantibodies in mouse sera were measured using microtiters plates coated with 1 μg/ml recombinant murine IL-17A (Rockland 010-001-B32), IL-17F (Rockland 010-001-B32) or IL-22 (GoldBio 1310-22). The plates were blocked with 10% BSA in PBS, washed, incubated with mouse serum samples, washed, and then incubated with an alkaline phosphatase (ALP)-conjugated horse-anti-mouse IgG antibody (1:500, Vector Laboratories AP-2000). Following washing, the colorimetric reaction was developed using the BluePhos® phosphatase substrate system (KPL 50-88-02) and quantitated on a microplate reader (BioTek Epoch) at 620 nm.

IgHV repertoire and mutation analysis. Live (7-AAD⁻) unswitched (IgM⁺IgD⁺) or switched (IgM⁻IgD⁻) NP-specific B cells (CD19⁺B220⁺NP₃₆⁺) in the spleens of immunized μMT recipients were sorted using a SONY SH800 cell sorter (SONY Biotechnology) and resuspended in RNAProtect solution (QIAGEN 76526). High-throughput IgHV repertoire profiling by RNA-Seq was performed iRepertoire, Inc. The raw sequences were processed and analysed using the IMonitor 1.1.0 pipeline. Zhang et al., Genetics 201, 459-472, (2015). With this pipeline tool, each sequence was mapped to the Mus musculus germline V-D-J sequences (IMGT, available online at imgt.org/vquest/refseqh.html) to identify the V, D and J gene segments, and the CDRs, such as CDR3, were also determined for clonal clustering. The sequences observed only once in a sample were filtered off to reduce the sequencing error. Subsequently, the sequences were normalized according to the number of cells in each sample. By comparing the sequence of each clone with the germline sequence, the mismatches of nucleotides were regarded as potential mutations. To eliminate PCR noise and sequencing errors, the first 25 bp of the sequences corresponding to the primer-binding site were excluded from the analysis, and the sequences were filtered if 3 successive mismatches were observed in them. Finally, the mutation rate for each IMGT position in the IgHV was calculated if the sequencing depth for that position was ≥10, and the frequency of each type of nucleotide substitution at these mutated positions were computed for each Ig isotype.

Statistical analysis. Results are expressed as mean±S.E.M. Statistical difference was assessed by t-test or Mann-Whitney U test as stated in the figure legends, unless otherwise indicated.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. A material effect would cause a statistically-significant reduction in the production of antibodies with increased SHM and CSR following AIRE downregulation, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1.-17. (canceled)

18. A method comprising:
modifying B cells to down-regulate a function of molecule autoimmune regulator (AIRE), wherein the modifying comprises one or more of:
AIRE gene editing and/or CD40 gene editing;
AIRE gene editing and/or CD40 gene editing, wherein the AIRE gene editing and/or CD40 gene editing comprises CRISPR-Cas gene editing, transcription activator like effector nuclease (TALEN) gene editing, MegaTAl gene editing, or zinc finger nuclease (ZFN) gene editing; or
AIRE gene editing comprising contacting the modified B cells with SEQ ID NO: 9 and SEQ ID NO: 10.

19. The method of claim 18 further comprising:
contacting a population of B cells with down-regulated autoimmune regulator (AIRE) function with an antigen, thereby increasing SHM and CSR during antibody production, wherein the increase is in relation to antibodies produced by a population of B cells with normal AIRE function contacted with the same antigen under comparable conditions.

20. The method of claim 19, further comprising contacting the B cell population with down-regulated AIRE with an adjuvant.

21. The method of claim 20, wherein the adjuvant is a Toll-like receptor ligand, a squalene-based adjuvant, alum, a STING agonist, and/or a cytokine.

22. The method of claim 18, wherein the modifying results in AIRE protein that does not interact with AID.

23. The method of claim 18, wherein the modifying results in an AIRE protein lacks amino acids 110-114 and 131-133 or lacks amino acids 101-180.

24. The method of claim 18, wherein the modifying results in AIRE protein that lacks its caspase activation and recruitment domain (CARD) and/or its nuclear localization signal (NLS).

25. The method of claim 19, further comprising stimulating the B cell population within in vitro culture conditions.

26. The method of claim 25, wherein the stimulating comprises adding CD40L, IL-4, IFN-y or TGF-β.

27. The method of claim 19, wherein the produced antibodies are human antibodies.

28. The method of claim 27, wherein the human antibodies have a dissociation constant of 10⁻⁸ M to 1 M⁻¹.

29. The method of claim 19, wherein the produced antibodies are non-human antibodies.

30. The method of claim 19, wherein the antigen is a viral antigen, a bacterial antigen, a fungal antigen, or a cancer antigen.

31. The method of claim 18, wherein the B cells are in vivo within a human, mouse, llama, chicken, rat, hamster, or rabbit.


SEQUENCE LISTING (13 sequences)

SEQ ID NO: 1 - protein, 515 aa, Homo sapiens [sequence omitted]

SEQ ID NO: 2 - DNA, 12382 bp, Homo sapiens
  source 1..12382: mol_type = other DNA
cgcgggggtataacagcggcgcgcgtggctcgcagaccggggagacgggcgggcgcacagccggcgcggaggccccacagccccgccgggacccgaggccaagcgaggggctgccagtgtcccgggacccaccgcgtccgccccagccccgggtccccgcgcccaccccatggcgacggacgcggcgctacgccggcttctgaggctgcaccgcacggagatcgcggtggccgtggacagcgccttcccactgctgcacgcgctggctgaccacgacgtggtccccgaggacaagtttcaggtgggctccccgcccgccccccgctgcccccaggccctgtgagccagggatagtccccggggaagttccaggaggaccccgcccctccagatccccaagcccctccagccttccccaactccctccccacaaggagccaggggcgtccctgatgacaagttagaagttggtccccttcccccagccgtccccacacctcacccccaagccaagggaatggcctccaggttcccccagccccaccctcaacacccctacaccaccacctgactccaccacaagccgaggagatgggcgtggagctgtccaggtcgccagcgcctctgcctgggagctccaccctctagtcatgatggagatgggcaggccgcagggtgtgggggaccatggcagggaccctcatgccaccccactgcaggagacgcttcatctgaaggaaaaggagggctgcccccaggccttccacgccctcctgtcctggctgctgacccaggactccacagccatcctggacttctggagggtgctgttcaaggactacaacctggagcgctatggccggctgcagcccatcctggacagcttccccaaaggtgggtcctggtggactcagccatgctgggggcctggggcagctgctgtcacctgctcagcccagctggactggaaccggagtggtgtttgaggagcccgtgggtgatgttccaggaccgtcttggatcctaagaggcaaaggggccaggcctcacctgtctggccaaggtgtccagttctggggcccaccctacccctggagaaaaccctgaggttgggaccctgctcctgcccctgagctgcagatgtggacctcagccagccccggaaggggaggaagcccccggccgtccccaaggctttggtaccgccacccagactccccaccaagaggaaggcctcagaagaggctcgagctgccgcgccagcagccctgactccaaggggcaccgccagcccaggtaccctccctgcaggggaagccagccagggtctccagtcttcccgggcttccccgggagcccacgccccctccccacccgggctcccacccactgggtgtggggccagcctgcctggggctgtgggggtctcctctgggtactagacccacacactggaccagcctctcagctccctcctgcctgaaggctgagctccccggagctggtgaagtaggcgggcgggtctcatttcccttttactgatgagaaaccagagcccggcaaagggactacccagcactggaccgccccctccacgccctcccaccgcgggcccctgcccaccggcactcacccccactgagaggggaggccaggctgcccccagctcccccattcaggctctcaactgaaggccaagccccccaagaagccggagagcagcgcagagcagcagcgccttccactcgggaacggtgagcggggcccagtgggagcgcctcccttctccctggccaggggcaaggggtcaggggtcagagcagggcctgccctctgagaccctgtcctaggggctggggacgtgctggcctggtgtgtcattccaagggcctaagctgcaccaccagacccaggaaggggacaccttgggtctaagcatgatcttgccagtcgcccctgcccccactgcaccctggttctgggacccccttctcaggcaccttctctgcccgtccactccctatccttcaggaccagcctagacatagcttcctccagaaaatcatccctggcccccagctgcatgcaggctgaacccttcctgtccccttctccttccttcccagggcactggactccagagaccccctatctccctgagggcagagcctaggaactctgtgtccctcccggcacaatacagggcccatgtcatgggggggtgggtctggtcattggtcatgccttcctatccattgtgccagctctgctgacactgccaccccccagcacacgcacacttgggtgcacacacgaacacacacattctcatgtctctgcacttacctgtgggctgtctgcacatggcagggctgggtcccctccttggcctgccctggctggaaggaaagggctctgcagcccagtgctgcctgcttctggcatagagtatgtgcttgggaacagtcttccccacgggtgaccccaatgggtgttccctttcccagggattcagaccatgtcagcttcagtccagagagctgtggccatgtcctccggggacgtcccgggagcccgaggggccgtggaggggatcctcatccagcaggtgtttgagtcaggtagacgctgtggcggggagatggggctgatggggagacccaggctccaagatggaaggaggaccacgcccctttgcatcctggtggtcccacagcagaccggactgttgctcaggtagccagagtttctgcctgtggttctgctgactttggaggaggagggtgagcactgaagtctccctgtcgggggaccttctgcaaggccagcggtccaggcccacatccccacccgggatgtacagcactccccagtcacctccatccatgtgcatgggccctcctgggccatggggttgcatccttagaaagttctgcctgtgctgctgagaccctccagggtatcggcattcttcaaccaggacagcctgtagcatagcgtccttgccccccataccctggccagcctgcagcatcctcgcccgccattccctggccagccgctgaccccatgcaatcaccagtgccatctgaccagggcacagcagggccgctggtggcagacccaccgtgccatcggggcattccatctcaagtccctgacacggtgtctcctcggtgctggacatgggctgggaacaccaagcacagccagggccctggtcttgcacctctggatggtcccaaggcccactgtgttacttcctaaggctgttggttaaattggcacaaactgggaggcttgaaatgacagaaatgccaacatcgaggtgtctcggggccacactccctctggaggctccagggaagaatccttccttgtgtctcccagctgctggtcattatgggggtacccctgtgctccttgttcctgggctcaggacccaccgctccagcctctgcttctgtggtctcacagctgtctcccacgtgtcctctttataaggacaccagtcattgaacttatggtccagtgtgacctcatcttaactaatcacatctacaaagaccctgatttcaagtaaggtcacactctgaggttctgggtggacgtgaacttcgggggacgctgttgaacaccctggtgtagatccaggacaatccccgggccccagactcgactggggtgggggcgggctggaggaatgcaggctgtgggaactccacctgtctctgctagaccccaccctggggcctacacgactgccaaggcaggtcctgctgggcgggtgagccaggaccagccggcatctcctcccaggcggctccaagaagtgcatccaggttggcggggagttctacactcccagcaagttcgaagactccggcagtgggaagaacaaggcccgcagcagcagtggcccgaagcctctggttcgagccaagggagcccagggcgctgcccccgtaagcacctgaccttccctggggagcctggctcttgatgccccccgccccaggaacagcgttgcctctgggggagtggctctgctgggggctgggggctgctgccgagagacgcctggtgccacagccatgtgcaccctcgctgctgaggctgcccccattgctgacgcccctcttccttgcagggtggaggtgaggctaggctgggccagcagggcagcgttcccgcccctctggccctccccagtgacccccagctccaccaggtaatgccctagaccacaggagaggcccctgtctgcccttgctcccctcgggtgggtcctgctgcctctgcctttacctgggcactcagggatgagcaccggggcctgagcccctacccacagggtacagctctttttctttaatagacagtatttttttcctgataatacgcaatggtaatagtttaaatgagtcagagaaagtgaggtcttctcaggctcttaagagcatggcgtttggtccaggctgtacccgctgctctcagctgggcccgtgggtgggccgggcgcccctgctatagccaggaggtcaaggatccactgggaatgccatgctcatctttcgtccccagcatggtttcttaatggggtagaagcagtgtggggggtgcctgccgtggtgggttacagatcttgaccacttggcaccaggggctctgtggggccctggcacttagcagtgacaggagccagtcctgccctgcaggagcacccgggctggtgggcgtctgggggattgttagaatgagtgaggtcattgccgtgcaggaccagcctagcctggctgtctggggggattctggaggaagtggtacctgggagacccctgaaggcacagcaggcaccatccaggcagggcacaaggacggtgggggctgcaggtggaggattcagcaggcgctgaggtcgggagagacctccctgggcctggccccactgccctgtgaggaagggttcatgtggttggtgtacagttccggggcccctggaacgcagcagcctgcaagaaaccgggttttcttcccaatagggatggccccggggggtgtctgttggagaccagatggatggggaacaggtggtcagggcagaatttcaggccctggcagcatgggagcagggcagagactggggagttcaggtacccagagatgctgctgggggagctgttttgggaaggaggtggctctcaggagggtgctgcaccccagcccagtctgcatgggcgtctcttgcctgtgccagaagaatgaggacgagtgtgccgtgtgtcgggacggcggggagctcatctgctgtgacggctgccctcgggccttccacctggcctgcctgtcccctccgctccgggagatccccaggtgagcctgcacctctgccagcgcaaccaggccaccccggttcacggccgcctccacccactgaccctgaagggaagccaccccaagcctctcccatccaagatggaaaggggttctgagtcaggtcactgggccgtggggccggggcctggggttttcccaccctgccacctgcctcccggtctggccacacctgctgcccagcctggacagctgggcccctgagggcagcaaagcagaacagaggcccagggcgaagatgccaccctgtccaagctcatcccaggctgcagcccacgcccccatgggtagccggcccccacccccaagccccaccccagagtcccactccagacagggctggggagcacagaggccacagagctgtgccccccagggcaggtgggagtttgtccaccaatgcacaggacgccgggcttagtgggggcgggaggcctcctctgcgttcacatcccggtgctccttccccacggcccaccgagccctgcccccattccaaccccacaggacgtggcagtctgtgggaggaagagctctgggtgcagtggggacccacgttcaggcgaggctctgccccagcccctgagtggccgtcatcaggccccctctcagccttgtgcctcatcactagaataaggggcacagtgggggtcattgctcggctcctgaagccgttcctccttgccgtctctttctgcccttgatcacctccccattctgctgggtgccattccccttaacaggtgggtcagtttagggaggcccccggcagggcccagccctgagaggcaggcaaagccaccagggctcgcaggtgttggggattcctggggttcatcagagagcacgccaaggggaccctgatcacgctggccagggccaccccacgaagggtaaatgtccccctgctgggctctcccttcctgtgtctctgcccatctctctgctgtgcctcggttccccctctgtgaaaagacatggtcggagccctggagctccacccgtgggtttggggatctgtcacccgctgtcttgttctgcatgtctctgactggtggacacacgagcagtgggacctggaggtgctccagctgcctgcaggcaacagtccaggaggtgcagccccgggcagaggagccccggccccaggagccacccgtggagaccccggtatggccacgccccctcctagccgggccacccctcctgtccacatggccacgccccctcctaggctgggccaccccctcctgtccgtctgtcccctggagtcctgtgggacaggactgccccagccatagcactatgtcccccatgcccaagcccggtccttgtggtctcctgcagtggagtccccatcatggttcctgtgggcctaaacccagctctcctggctgcgggtccaccccggggggcactatgagcattgataacggccccggaagatgtgttccttgttctgctgctgtgagggtagtaggtctactgtgcacagacccagtgttccctctgacagccctgagggccagggggccccccgtgtgtagacgggggaggagggaggaccacagagccaggaagtgccacagcctttcccactcagtgtggacgccttccaccatgccagccctccgcccccaccatgccaggcctctgcccccaccctgctgccctgggtttcagggtcccagcagtcactgactcctgggtggtgccgggcaggcgcccgctgcccctctgatgctgacccttgggttccagctccccccggggcttaggtcggcgggagaggaggtaagaggtccacctggggaacccctagccggcatggacacgactcttgtctacaagcacctgccggctccgccttctgcagccccgctgccagggctggactcctcggccctgcaccccctactgtgtgtgggtcctgagggtcagcaggtgagcggggagtgggggtcagggtgggctcttcaaggagcccaggacctacggggcggatgaattcacctgaaacaggaggagagggaggccaggcgagaaaggctccgggaggcacagggcctggggctgtggggggagcgtggggggctgcggggggaaggggacgctcctagacctccactccagctcctggccctgggcattactgctccccccacaaggcaggacaatgaagggggggatgtcccagcacacgtgggagccctcccctccctgcctcaattcccttccctgcacccctgtgggcaccgcctttcaggagactcccgcactcagccccaaaggaggccaggcccgccaagcaggagagaggtgcgggcgccaggcttgcaggcagcagcctgagggtgcttgggtcgcccctgcctcctggggatgggactggtcccgctgtcctgcagcctgcgtggcaccgtgaggctcctcacttgcgcctagacccgccgtccagccctgggtggtcccaggggagagcgcacagggctcgggttcgggttcagctacatttcccccggccccccgcgtcaccccgcgctgttgcctcccacagaacctggctcctggtgcgcgttgcggggtgtgcggagatggtacggacgtgctgcggtgtactcactgcgccgctgccttccactggcgctgccacttcccagccggcacctcccggcccgggtgagtgagcgtggtcggcggggaggcctgaacccacacccacaccctacaccccaccccacactccccacccacatcatacagcccacaaccacaccccacccacaccccacactcccacccacaccttgcaccccaccccacacccatgccctgcacccacaccctacactccacagccacactccaccacacccccacccacaccctactccccacctcataccctgcacctcaccacactccacagccacaccccaccccacaccccacactcccacccacaccctacacccaccccacaccctacacccaacccaaacccacccaaacccaccactcccactctccacccacacccacaccccttcctcacaccccacacccccatcccccactcaccacccacgcccacaccccacaccccataccccggaggtggcactcctgctcccccccagggctggcagcccctcatcctctgctgcaggacgggcctgcgctgcagatcctgctcaggagacgtgaccccagcccctgtggagggggtgctggcccccagccccgcccgcctggcccctgggcctgccaaggtcagtgccgcaggggccctccatgcatgccggtgctgggggtggggaaccccttgggttggtgttgggggagcacatctcagggcagaccctgggtgccagcttcgagggcttgcaccagacgcactgaccatgtgctcattatctgtagaaaatatttcccctttaaaccaattctttttggcaacttaaatatagttaaaaaggaagctccccccgagggttggtggctgacgtcacggttggctgtgtggccgcctcacagcatgagcctgagagtcctgccagggctccctggtggggtgaagggagagcgggagcgcccggcctgcaggagcaaacccccaccctgtctgacccctccaggttgtctcacccccagccctccctggggccaggatccaccccactgtgtggccagagccctctcagagaggcaaagtgaccccgggtccagccagtagctcttcctgtcctcctgctccggggtcagagaggacctgggtggcgcggagacccctgactgctggggcggctgggcttgccctggagctgggtgtgggggaggcccgagtcgctgctgcaggagcctccgggggggtggcctcttgccctgaccgtccccagcagaggcctcctgagcacatcctggccaccgaggagcctttagggatcctggggtgatgacacgtcccacctgctccactggcccatgctctttcccagctgtgcctccgccccgtatacaccgtgtgggtgacaggccaccccggcgtggtactccccaggagggtgacagcctaccccagcgtggtactccccgggcaggtgacaggcttccccggcatgcaggctctggcctggcatggcacaagcctcagacccagccctgcccttggggcttttgtggaacagtggcgtggcccacagctgtcactgtccccttccttctagaagcctccctcctcacaccacccatctggagtcaggagcccagccgggcatatacgcagatgcccctccctaaccccaggcagctttcctgcaactgctcccgcagcgggtacctcgtcattaacctcctgggttctgtctctgaacagcagagacctctttcttgtcatcgtgatgtgaaatgtaacgccatgtcagaggaaaagttctggctggccttggcctcccccctcagcctgcccccttcctccagggtggttggacgtggccccagaccccatcctgagcagctctcccaccccctgggagcatccttaggaccggggagcatccaggggctttcccctccagaccgggcagcccctccctcagccatgcagggctgccgggcctcgcagcgccagtgttcacccgagtggaggagctgggatgtggctgtttggggccacaaatggggaattccacagggttcaatgtaatatggtctcctctctgctgggggtgcctgcctggggaccttctcccactctggtcgctcacctatagtgtgggctggccctggtggtgcttgtcgggggcgggggtggcatggaccaggcactttcctctctgggcctcagacttcccctctcagagtgggactccttgctggttccctgagctccctcgttttccccaggaggccacacagtgtggaggctgtctgggggccgtgggcagctggccgtgggcaggaccctggggaggcagccccagccccatcatgcccacgcagccctgtgcccccacccccagtggagctgggtgtaagaattcccatctcagtgtgggggaaacacccccgcggcccctaggccctgcggcctctgtacccccaccagggctgtgggagttgggctgacctcttctctttactgggttccaggatgacactgccagtcacgagcccgctctgcacagggatgacctggagtcccttctgagcgaggtaacgcctcccctggcctcctggtgctcctccactccccctcccctgcctcagccggcacccaggctccccactctgggggaggactgccggcccccactgctcttgagccgtggaaactcaggctgtccctgctccacccaccaggagccccagtgctgctgagcacctggcaccccccacaggagcccccctagcccccttgcaggagccccccccggcccctccccctgcgggagcccagtgctgctgagcgcccccagcccctccccaacaagagcccccacacggcccctcccctgagggcctgcaccctggcaggcagaggctcgagcaccaggctcaagatccactttcccagggagggtggggcgtgggagtggggggggggtcccagaccccgtccctctaagatttgcttgcccctcccaactcaggcctctctacgctaagatgggcaggtagaatctgtggggaaaatgtgacttttaagggctctgtctgtttttgccaagaggataagctccttcagcctccacgggttctcctcagtgtctgatgtggcacccgggggtcccagctgaccatggggcaggggttctgccctgtgcagtggccgtgccccacacaccctgaccgtgcaggtgtctgcagagccccagggcctgagagtgggccagggggcccagcgctgggtaatggagctgcccctctggatggggtccccgggtatagctggagaaatgagcgacgggctcacagcctctcccgggtggcggtcttattctgctggcatcgtggggcccgtggccccatcctgtgggagcatcaggctcctgagcagaataagtagctggccccgacccccccaccctgaaggagccacccgaggaggcagaactgccatgaactgccatggggatgtgccctgggcttataggatgtggtgaagtacacaggacagggtcctcggtctggcctgtgccatggggaccttgggcctcagtttccccacctttgatggaatacggtgaagtgcacaggacagggtcctccccagactggcctgtgccatggggcctcgggcctcagtttccccacctttgacttagagggaaggttggatggtgacttcttgtaacgatggccatgattctgtggctgcggcgggggcgcacctggaggttctcaccgtcactctgtcccgcagcacaccttcgatggcatcctgcagtgggccatccagagcatggcccgtccggcggcccccttcccctcctgaccccagatggccgggacatgcagctctgatgagagagtgctgagaaggacacctccttcctcagtcctggaagccggccggctgggatcaagaaggggacagcgccacctcttgtcagtgctcggctgtaaacagctctgtgtttctggggacaccagccatcatgtgcctggaaattaaaccctgccccacttctctactctggaagtccccgggagcctctccttgcctggtgacctactaaaaatataaaaattagctgggtgtggtggtgggtgcctgtaatcccagctacatgggagcctgaggcatgagaatcacttgaactcgggaggtggaggttgcagtgagctgagattgcgccactgcactccagtctggtcggcaagagtgagactccgtctcaaaaacaaaacaaaacaaaaaaaccacataacataaatttatcatctcgaccacttttcagttcagtggcattcacatctcatgtaa

SEQ ID NO: 3 - protein, 552 aa, Mus musculus
MAGGDGMLRRLLRLHRTEIAVAIDSAFPLLHALADHDVVPEDKFQETLRLKEKEGCPQAFHALLSWLLTRDSGAILDFWRILFKDYNLERYSRLHSILDGFPKDVDLNQSRKGRKPLAGPKAAVLPPRPPTKRKALEEPRATPPATLASKSVSSPGSHLKTKPPKKPDGNLESQHLPLGNGIQTMAASVQRAVTVASGDVPGTRGAVEGILIQQVFESGRSKKCIQVGGEFYTPNKFEDPSGNLKNKARSGSSLKPVVRAKGAQVTIPGRDEQKVGQQCGVPPLPSLPSEPQVNQKNEDECAVCHDGGELICCDGCPRAFHLACLSPPLQEIPSGLWRCSCCLQGRVQQNLSQPEVSRPPELPAETPILVGLRSASEKTRGPSRELKASSDAAVTYVNLLAPHPAAPLLEPSALCPLLSAGNEGRPGPAPSARCSVCGDGTEVLRCAHCAAAFHWRCHFPTAAARPGTNLRCKSCSADSTPTPGTPGEAVPTSGPRPAPGLAKVGDDSASHDPVLHRDDLESLLNEHSFDGILQWAIQSMSRPLAETPPFSS

SEQ ID NO: 4 - DNA, 13589 bp, Mus musculus
  source 1..13589: mol_type = other DNA
agcaccacgacacccaaggaagggagaagggaacgcaagcgcgcgtgggccagcagggggcgccgaggcgcagcccctgtgaggaagatggcaggtggggatggaatgctacgccgtctgctgaggctgcaccgcaccgagatcgcggtggccatagacagtgcctttccgctgctgcatgctctagccgaccacgacgtggtccctgaggacaagttccaggtgggctccagtcccgcccccggtgcctctcattctccccactcctccacccgcagactaggtgttccctcccaacctcagccaaaacccatactatacccataccctccccctaccagccaaggagtggtcccaagccctcctcaggagacctctccagatcaagtcccaggtgagttccctaacctcacaccctatgccccctaactgctccagggcccagggatagacaggaataggcaagtctcccttatcccaaagaggcaggagttggagaatgatatgcccaggtgcccaatgctgtcactgcaggagacgctccgtctgaaggagaaggaaggctgcccccaggccttccacgccctgctgtcctggctcctgacccgggacagtggggccatcctggatttctggaggattctctttaaggactacaatctggagcggtacagccgcctgcatagcatcctggacggcttcccaaaaggtgggcgtgtgctgattgatgctggagctgatgctcagccaatgggtagcatcggggatatggatacaagtcggcccatgttttcagggagccactagaacttgggcagatcctaagaagcaaagggcagaggtctgctctttctcgtcctcaagagtgccccattctagagctcaccctgaagataaggctttaagacaggaccattgttcctgcccctgagctgcagatgtggacctaaaccagtcccggaaagggagaaagccccttgctggtcccaaggccgcggtactgccacccagaccccccaccaagagaaaagcactggaggagcctcgagccaccccaccagcaactctggcctcaaagagcgtctccagcccaggtacactcaagaggagctagccagggttgctgggccctccccaaccggctcttaggagcttctgtcttactgacaccaccccagggccagcctgccagggtcacagagtcacctctgagccctcagacctgagcattggaggaggcccacagcctctcagcgtcttactgtcccaaaggctgagtttctgggcggtgaggcaggcaggtggttttgatttcctttctgttgaagaaggaaacagcccatcacagcttaagaaccgtcgatctgacccttaccagctgctctctctcccatcctcactttctaccctggatccgtcaacatgaccccagcccagaaaagtgggcccaggctgcctctacctccccttcgcaggctcccacctgaagactaagccccctaagaagccagatggcaacttggagtcacagcaccttcctcttggaaacggtgagttaggccaagagtggaggttggaggaggtctgatcccattgacctcagctggatggcaaagccagagaaagatagggactccttagatccaactgtcttgccattctcctacccacaatgccctgggtgtctcctcccagacctctgcccattttaatgctcccaatcttctagccagcccagaaaaagaaccacaaggaaactatccctgttcctcagctgcgcccaaccttgaccacacccacccaccatccaccatccacctgtgcttcctggtcctcaccccctgatggcctaggaactctgtgccccagaataacgcaggtcccacgtcaccatgagattcttgtcaatctgccattgggctcaacatgaccaacactgctgtccccacggccgtgtgctcatgcacatacgtctacttgtgtcaaaccctctccaggaattcagaccatggcagcttctgtccagagagctgtgaccgtggcctctggggatgttccaggaacccgaggggccgtggaagggatccttatccagcaggtgtttgagtcaggtaaatgcatggaagcaggctgccagggagacccagatttcaaaatggaagggagtgcttctagagcatccatggccgtggctgagggcaaggcagccagtgtgcttcattcaggtctgctggctttggagcccagtgctgatgtggaagactccctacatgggtggatcttttgtcaggccagtggttcatagtcacattcagccatggaatgccacctcttccacgtcaaggggtgctgctagtcacaggggacatcctaagtttccctgtgtgctctagttctgtcaagagaccatagtgcccactaagacagcccaccacatcctactgccctgtgtcaggtatctatgtcctgtccagtcccctcactctttgtccaggttcctcccacaattgcctacctacaaggctggctagtggggtcaccttatacagccacccagatcatctgaacaagtcagagctggggccagacacactcaccattgcagaatctgctccacagcacctcctctgtccgggacactggacttggatgccatggacagccagaatggcctgggagctcatgccagccctagtcagagcagccccaagaatcttgtctgactcttctggggttattagaacaaaaagccccaggcttgtaacaacaggttccaaagtcaaggtgtgggaagggacacgtcccttcgtgggctttggagggtccctctgttgcctctagcatgaagtgctgttggaatgcttacttctgagaatagcccctctccaatctgactattcctctctgacttcacctttctttgtaagaacattggatttaagggctagtatgacatttttaaaaaaagatttatttaatttatatgagtacactgtagttgtcttcagacacaccagaagagggcatcagatccccattacagatggttgtgagccaccatgtggttgctgggatttgaactcgggacctctggaatagcagtcagtgctcttaaccactgaaccatttctccagcccgtatgacatctttttttgttgttggtttttctgtttggttggttgcttcttttgttcgtttttgtttgtttgtatttgagacagttcctctgtgtagctttggctgtcctggaactcactcactctgtagtagagcaggctggattcgaactcgagtagatctgcctgcctctgcctcccaaggattcaagatgtgtaccaccatgccctgctatgacatcatcttaactaactctctcgccaaagaccctgtttccagtaaggtcccgttctgagcttgtgaggaagggcactgtttaagggtacagtcaccagcaaagaatccctagctgtacccagccccggttctgccagacccccagggtgagctcattcagtctatctctctcccaggaagatccaagaagtgcattcaggttgggggagagttttatacacccaacaagttcgaagaccccagtggcaatttgaagaacaaggcccggagtggtagcagcctaaagccagtggtccgagccaagggagcccaggtcactatacctgtaagccttatccagcatgtccatttagggggagctgggccttccttccatagcctcccctcccctcccctcccaaagaaagcctggagttcttcccgagggtgggagttgcttcccagtggtacttggtggccacatagatcttccctgaccctggctacttcgttaagaccctgtgtctctcatagggtagagatgagcagaaagtgggccagcagtgtggggttcctccccttccatccctccccagtgagccccaggttaaccaggtaagtcccaagaagggggtgggggtgggggaaccaggatatggagggcagctcccttcctcttctctcccttctctttcctccacctcctcccactcagctctttcttggaagttttcaaggatgcatattaggagatttccaattagtcacagcaggtgagctgcttttaaaaaaatcacacgttcagctggttggtatatgcctttaatcccagcccttgggagacaggggcaggcagatctctgtgaattggaggctagcctggtctacagagttccgggacatccagggctacaccaagaaaccctgtcttgacaacaaacaaacaaaacaaaacaaaacaaaacaaaacaaaatcacacattctgtccagatgacaaaaagcacattagcttcgggtcgggtagggctgcagataaagcctgagtatggggattctctgagggtatcaagttatcctctcctccccagcaccactgatgtggttgtgcctgcaatcattgattattgagtccaacacctcagagctaggtactctgctggccccggacaccaaaggttgaaggtccagttctgccctgctccataaaagtgccctggttattgggggcctatggacgtttcccatgtgctcactgtgtgggcccccacaacctggcatctgggagcttcttgaggaggttcagactcagaactccttccatcctgataaggtaggggatggggaatgacaggaggtacctagggctaacgggaaaccccgtgggcccacccacctgctgtcctattaggtgagtggtagtcaatccagggaactgtgggcctccccactctgtgggttgtcagcctgggctacaccgggactagctatcaggaggcagatcaactttacttagggtgattcaaggcttttaaaaaaacaaaagattaatttattatttattataagtacactgtagctgtcttcagtcgcacggatggtagtaagccaccatatggttgctgggatttgaactcaggaccttcagaagagcagtcagtgctcttacctgctgagcccaattcaaggcttataacgttttgggggggggggtctgagggtaggaatatctaggatgggcaaaggtcattggctgggggcttggggtacctaaaatgtctccttagcaagggaaagcatttcggaaatctcaggtgctgaatgaatgggcgctttgtcaggagaaaggaagttggtcctgtaatttaactgaggctacgtgacattcttcatgcatctctgagggcattcctaagcctggggggggggggggcgggggcggggcttagaattccccagacaaggtcaaagaaggagaaatcctgttaatgaagggagtcccccatattgcgctgagctcagaagctatccagtcatggtgggattttgatcttaattagcagagttttcccactaggaactgcctggcacccagcactccttccagagagggacacccccctaggtctgtgtggaacctagtacttcagagaccttgcagagctcaggcaggggccaacgagagaacccagggggaacctgggagtcagtccttagtggtgagcattgtcccaagtgggtctctcaggtggatgctgtgttccattctgggtccctcttgcctggtcagaagaacgaggatgagtgtgccgtgtgccacgacggaggtgagctcatctgttgtgacggctgtccccgggccttccacctggcttgcctgtccccacctctgcaggagatccccaggtaagcagacctctccatctctgatccatcaccgtccttatccgctgacattgaggaagcctagaagccttcacagaagacagtaagggcccttcagtaagatctgtggaatgaatagggtactagctatggaaatggtaggattctctggccataccgaaacctatctgtgttccctgcctcatggcccactcagggccaacaggccaaaaggcagccataacatgatgtccccaggcaaaggtggccatatttgggcacaggccttttcctaagcacactgtacccccaccccctcaaatctcactcacacaatgatgtactgttacccctatcactgactgcacaaccctctgttctgaggagaggtaagcagcagtctgtgcgggctagcctgggctctgacccaggtctctctgatcctcttccaccagggtcctttccctcatctcccctttcccactggtccatccctacaggactgtgggtgtgccaaggatggaaacagtgtagctgagagtcatgcttatctcccctcccccactggcccatccctataggacggtttgtgcacctaggatggaaacattgtagctgagagagcccacagtaatgccaggcctatgacaagcaagcacaggtgctgaagggtactgggcatagagtattcttaagaattagcagagacccgcagggcatggtagtgcacacctttaatcccagaactcgggaggcagaagcaggtggatttctgagttcgaggccagtctggtctacagagtgagttccaggacagccagggctacatagaaaaacactgtctccaaaaaccaaaaaccaaaaaaaaaaaggattctagggaccctgactctgtccagagccatgctcccgaggtaaatagcccacattctattgaactgcccttatggtacctaactcttttagtcccgggtttggcttcccacctatgaatgagcatggtgacacagagggtcctcaagccatccttgccatacatttggggaagggggggtgtctatggtctctactttgtctggcaagcctgtgactagtgacctcctacacagtggcctctggagatgctcctgctgcctccagggcagagtccaacagaacctgtcccagcctgaggtgtccaggcccccggagctacctgcagagaccccggtatgcccatattgggtcaccccctttcttctctctgactctctacagtcctatcttctggcttcacctgtgagtcctgcatgcccgccatgctctgttctggtgaattccctgtggggttgaggggccaagggattaaaaacagcttcccaagctggctctgtccctccagactcaccactgccaatattctccaaaagccttggtagctctcctgttagaaacctagtttgctctggcttggccctggcttttgctgagaaccatggaaccagccacacactcagcctttccttctcctcctcctcctcctcctcctcctcctcctcctcctcctcctcctcctcttcttctcctagttttattttttaaagatttatttatttattatatgtaagtacactgtcactgtcttcagaaactccagaagagagcttcagacctccttacagatggttgtgagccaccatgtggttgctgggatttgaactcagaacctttggaagagcagtcagtgctcttaaccactgagccagttctccatcccttttttttttttttaattttgttttgttttatgtgtattgatgttttgcctgcatgtatgtctgtgtgagggtgtcaagatcccttagaactggagttgcagacagttgtgagctgtcatgtaggtgctgggacttgaacccgagtcctttagaagagcagccagtgctcttaactactgagccatctcttaagcttgtaccaagccttcccaacctcagacccaccacagcagcccagtcctgactcctaggtgttgctgagcagtaccctgtggcccctgaatgctaacccttgaattccagatcctcgtgggactgaggtcagcttcagagaaaaccaggggcccatccagggagctcaaagccagctctgatgctgctgtcacatatgtgaacctgctggccccgcaccctgcagctcctctgctggagccttcagcactgtgccctctactgagtgctgggaatgaggggcggccagtgagtgaggagacccctagggcctgggtgctatctttggggaagaggggctctggacctacgggatagttgtatgtccaaaacgggacctctggtggactcccggggctaggaattagacggacattcctggggtcaggggagggctctgccaagagacacttgttcatataatagtaacacagggactgtaggggagagcaccaagggaactctccagctgggcctttgagatggctcagtgggtaaaagggtttgcagccaagatggaagacctgagttggattcccaaaactcacatggtggaaagagtgactcggtctgcaagtggtcctctgatctccatgtgcatgaccaccaataaatagtgaattagctagaaagaagggaaggagggaagtaagacaggcaagcaggcaggcaggcaggcaggcaggcaggcaggcagacagacagacagacagacagacagacaggcttagcttggttatccagatgatacaccctcccccatcatgaagcaggacaacaaacatctcgaatccacagaccctgtccacttctgttggttacctttctttttttctttttcagagtccccacttttaaatttaattaattaatttatttattttacttattcactttacatcccgctcactgccccttccccatcatcctctcgcagtctttccccaccctctccctcctccaaagctcttctggagtcccacatttggtaggatcagagctggactcccaggatggactggtccgtggaaagcagcttggaaaggccgctttctgacccctgctcccctcgggacatccagaactcagaatttacagtcccactcacgcgacttgaaaccctgaggtttcccagttaacccgggctggtggggtgagcaggacacgggctgggttgccgcccatgttgcccctgcagggtccagcaccaagcgcgcgatgcagtgtgtgtggcgatggcaccgaggtgttgcggtgtgcacactgtgccgctgccttccactggcgctgccacttcccgacggccgccgcccggccggggtgagtaagggggcaccgggtggcagagtagccagcgatctcacccaccccgaaggttctccgagccagtgagcttttcccacttctctcggacaggaccaatctccgctgcaaatcctgctctgcagactcgactcccacgccaggcacaccgggcgaagctgtacccacctctgggccccgtccagcacctgggcttgccaaggtcagtgtctgctcagtccaggtgagaccctgtgggagtggaggagaatttaaacccatatccaataaccgtgtgtcccattactttttgtttggttgggtttgggggatttgttttttgtttgttgttttggatttttgttggtttgttttgttttgttgaggtactacagatctctggctgtctggaacttactcgtagaccaagctgaccttaaactcagagatccacctacctctgcctcccaactgctggaattaaaagcacatgacccttattcctggactaacttttttgtttgtttttaaaaattaatcatttttaatcttttttttaaagatttatttattattatatgtaagtacactgtagctgtcttcagacacttcagaaaagggagtcagatcttgttacagatggttgtgagccaccatgtggttgctgggatttgaactctggaccttcggaagagcagtcgggtgctcttacccactgagccatctcaccagccccccttttttatcttttaagatttcttttttatttatatgagtacatcgtagctgccttcagacacaccagaagagggcattggatccccattatagatggttgtgagcgagccactaagtagttgctgggaattggactctggaagatctctccagtccttgtttgtttgtttggttggttggttggtttttcaagacagggtttctctgtgttcccctggctgtcctggaactcactctgtagactaggctggccttgaactaagaaatcagcctgcctctgcctcccaagtgctgggattaaaggtgtgtgccaccacttcccagcccttgtttgtttttttgtttgtttgctttaagactattgctgggatcaaaggtgtatgtcactaattctggctcctcaattacttttataaagtgtttttcaataaacagacaacttattttgataagttgaatctatttaaaagggaaaggctggaagtggtagggcaggacattaaccctagcccccagcagtaaagataggttggtctctgtaagttagaatccagtctggtctgtatcgtgacttccagtttagttagggctacgcagtcgaaccctttctagacataaccaagtattttaaagggaaaccttccaaatagctatgaactcactgttggtactgggttcctgggagtccgtgggcctcagagctcagagctctgatcagggagctctggacctaagagcagttggcttaatttgaaatgagacgactgggaggcacccaggggccagatcgtctgcagaacaggtatcagaatggctgggacctccagggcggccaactattggcctggagagagagagagagagagagaaagagagagagagagagagagagagagagagttggggagcagagccaaacaaagctgctggcttttgatgaccccgagagccacctacccaagtcctctacccaggtggatctctatgtcacctccatttttactaaccaggcagggaccgagctttgtctggaggcccaaggaggaaagggcagtatccacctctatccacttgccattgcataatactgctaagacagacatggaccccaggcttgggcccctagctgtctctcaggttcatgggtaaaatggagtgatcttgtaaagatgtgttatgcaaggtggcagagtgtatgcgcagctcactgctagattccagctttccctgataaattgcccccctagttgcaggttgaccttatctgattgagaacatctgggaggctgcatcagaatctattgctcctaggattctactggaggacccagtaaagcatggggcaaggtttgctgagcctttaaaacagtggtcctcagtgatcctttggtgccaaatgatcttttcacaggatggcccaagactgttggaaatatctgagagttgggattcgtaacagtagcacaattacagttacaaagtagcaacaaaaataatgttatggtgggggggggtgtcatcacatcatgaggaactgtattaaagggttgaagcattgggaagattgagaaccactgtcctagagtctagccacaaacagtgaaagtcacttctagaatgaagacgaggattgagcaatggggcctccactggcttcactgtgaactcctgacagagcctgccactccctgactggagctctgtcactatggccagggcaggtccaactaagttcagctcatgtgactagtgaaagcagagagacactggctgggtgctagccaccgggtctctttaggggctacagggaatatataggacagattgttgccctcagcctgcctgtgtccccagcctgaggtactaggtgtgagtctgctctgtggtggagtacagtctactgccccaagtcctgcgtctggtccctagcctgctgctaggttgggttgactattgattctctgctttggttccaggtaggggacgactctgctagtcacgaccctgttctacatagggacgacctggagtccctcctcaatgaggtaatctgtcacctagtctgggctgtgctcacagattctttgctgcccctagtaggcagcaccagcagaccagtgttaccctgcccccatagctgctcttcctagaagagcttgtttccatgtagggtgctggcctcctccaggaacctcacatctgggatcctcacatcaagacctcagagccagggggtttaagcccttgacttttcagggaaagctgggagggtcttccaggggcctctctcttacccctcccaactcagttttctgtttcttgaggtggataggtggcccaggtctttgattcccagtgcttgaggtagatgtaggaggactgtgaggtggatactaatctggactacattataaaaccctatctcaaaaacctgaggggctacagaaatgcaagagcattgttctttcagaggacccaagtttggggggggggggtcccagcagccatgtcaggtgacttttaactgtttatcattccactccaggggatctgaaacctctggcctcctcgggcatctgtacacacatcacgcacacacacatacttatacacacagacacacatatacacataggtgcatacacacatgtacatacccactttttttttttttttttttttggtttttcgagacagggtttctctgtgtagccctggctgtcctagaactcactctgtagaccaggctggcctcaaactcagaaatccacctgcctctgcctcccaagtactgggattaaaggtgtgtgccaccactgcccagcatacatacatacttaaaagtagtaaattcccagaaaccacatggtggctcacaatcatctgtaatagaatctgatgccctcttctggtgtgtctgaagacagctacagtgtgtgtgtgtgtgtgtgtgtgtgtgtgtgtgtgtgtgtgtgtgaatattatgtgtatatgcatatatataaattaaaaaatagtaagaatgaatttcttctagaaaatgaaatagggccccatctattgcaagggcacaaactcctttggtcattcccataccccagggagcccacatgccttcttcagtgtccactgacatttagggagactcaactggctgagggacagagacctgccctgggcagagggccatgcacaggcagatctgcagaagcacgagagagagagagagagagagagagagagagagagagagagagaggatccagcatgggtggacactggaaaaactgaggaccttctccaggacttcccagtcctgttggcagagaagaaggacccagcagaggaagccacacccagggcccacaggctgctctagaggaggactggatcaagagacctcccttcactggcatgtcccatgggccctagctctcagtccccctccttgaactagcgggtcaggttgggtgataccttcatgtcaagatggctctatggaggtgcgactgctctaacctcccgtgtcactctctctcagcactcatttgacggcatcctgcagtgggccatccagagcatgtcacgcccgctggccgagacaccacccttctcttcctgatgacaggtggcccaggaaggggtgggcagcacagcattggctccctccccacccagccccatcggatgaggcactctgttctgagaggcctgggctgattaggaccaagagctggcaggttctggcctgctggactcagcttgcagatggccctgatctttgtagagatgcaaggccaccccatatcctggaattaaagtcacttctatgtactttgaggta

SEQ ID NO: 5 - protein, 203 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTRSPGSAESPGGDPHHLRDPVCHPLGAGLYQKGGQEANQ

SEQ ID NO: 6 - DNA, 11504 bp, Homo sapiens
  source 1..11504: mol_type = other DNA
agaccccgcccctttcctgggcggggccaaggctggggcaggggagtcagcagaggcctcgctcgggcgcccagtggtcctgccgcctggtctcacctcgctatggttcgtctgcctctgcagtgcgtcctctggggctgcttgctgaccgctgtgagttgtttttgccccgaccagacgggagttgggagtggggaatgagaaggaaagggaaggaagacttcggggaagaggccttcctggctgatttttgtgggggcaggagggtgggtgggagctgggcaaggtgcccccgctcctggctgaatggggtgggctgcctctctcttctcccgggctggggtcccgggagcggcctacaggggccgctcagggaaggcactggctgcccaagcgtgcctagacggcctggacgggtttagggagcctcagaggctggccacacagagactggtagggggttcagagggcgggaagtgaggcggaccaagggaaggggcgggtctggcccgtttcctgtccccttcttattgtggacagatgccagcctctgtaagtagttatcatctccttgccagctggggctgccttcttccagggcatcttgtgggaacaagagatgggtgcagaggcccaggtacttttgtgagaaggcaaggagcttttaacatcgccttccaccccgaaccgtatcttgggtgttccaacctaggaggaatccccagggctttgcctttttctcctgaatttaagatgacataggagacccctggggagatgaacagtttatgggacacaataaagggttaggagaccagagttctggttggctctgacagggctggtgatcagagggctggagaaaccaggggtttctccaggcaccagaggggctcagagccaaccaagcatatctccgggattttcagaagcctacacttgactcactttttgtttaaatgtatttttgtagttcctcattctggaggctgggaatcccccaagtacctggctccttcatcccagcccctctggcctccccctactttagagggctgtagattcctgcctgaagcctgggcaggaatgacccatggtatcaaggaaagcaagggaagcagcaagggaagagagggagtggggaggctgctttggtcccacagctttcactttcacctgaagcaatggctcttagggaacagggaggcagggggagggcggagctggaaagaggtaaaggggggcccttgtggtaggagtggagaaagagccagaggaggtggggtgaagggtgtgatccaggcttctcaagagcagagtttgccctcataactcccaactttggctccaggtagaggctgggctgtgacaacaatgtcagaagctatctattgagggcttcttgtgtgtcaggctctgagccaaacactgcctgttttctttgtctgatttctcacaactcccccattatacagatgggcaaattgaggctcagaaagggggattgtcttgccaaaggtctcatagctagctaatggaagaacctggttgtgaatctacatctgcatgattcccgagcctgcctctcagatagtgagagtctccaagctctggtcctgagctgttttgtggcagaaggaccagaactatggggagtgagaactggagattgacagacttttaggggagcgttttatttctcatgtgtttgaagatggtatcaaggactttcctatctttgggagtgtgggagctccacgttcacaggatggtgtcttgcaatgagctggtggggggcagtagccttttctacttcctttcccattttgggtaagacacatttctgtaagtaatttgctgagatacccaggttgaatgagagccaccagttaggtaggattctggacagccagccaggtagccgggctgcttgccatatatcatgcaagcagaaacaaatgaatgatgattaaaattgccatttaatgagcacctactatgttcctgacactgtgctaggccatatacatgtattctttcttatcttcgtaatccaacctgcagggcaggcattattactcccattttagagatagagaaactgaggctaagagaagcaaaataactagtaagtgttacaaagtcaggactggagtctaaagctgtctgactctcaaacttgtgttcttttcactggctgttcccaaactgtgggacagttttaaggagcacatggacatagaattaaacatacacttactttacagttcttttaaaaatccttctcattttttcaaagaggaagtctctggagctagaatagagttaatgcctctcaaaggcttgctaatccttcttttaaaacaaaaatcaagagcaggcctgggagggccttcaacaagcaaacaaccagctgggttttaataaccttgttttgtttccccagaatttatttttagggttaccttttatttatgagaagtgatactggttcttgtctcttggcaatgatgtgaggtttacatttaaagtaaatgtaccggccaggcacggtggcttgtgcctgtaatcccagcactttgggaggccaaggcagtcagatcacttgaggtcaggagttttagatcagcctggccaacatggtgaaaccctgtctctactaaaaatacataaattagccgggcatagtggtacacacctgtaatcccagctactcaggaggctgaggctggagaattgcttgaacccaggagatagaggttgcagtgggctgagatgatgccactgcactccagcctgggcgatggagcgagactctgtctcaaaaaataaaataaaagtattgaaattaacaataagtaattaatagcatgggtggtacctggatgtagtaaaatggtgaagatgaaacacaagttgatggagagaggagcattgagacctgagttctcatttggactctgtcactgtgagactctgggcaagtgaccctcctctttggtgctcagtctcaactatctgtaaaatgaaagtgtgagtttacccttccagctttacattctagcattttatgagggaagggctggatgaacagatgatgaggagttggaggaagaaaacatgatgggctttggaaaggagcaggaagggaagcagaagaataggaggaagaggccaagtgctaaacatagccccaaacagcactgggaccagctgaagtcagccagcttcaggactccaggggagctgctggagtccccatatcctatgggatctttgggaagaggaatgactcaggcatcaagccccaaggaattctgttctgttcagagaatattgtgagtttacagtaccattgctttgtaaaaataccagaatgattctctgggtgcgattataatcagctcagttgacaatttacttgaaaacaaacatgccaaatatcatgcaggttccactttctgttttgacttgcacttcagtttgcagcctctgtcctggatgacttttacctttctgctgaagaagttgcaacggagatttcaagatcccttcaaattgcacaattctgtttttaggtccatccagaaccacccactgcatgcagagaaaaacagtacctaataaacagtcagtgctgttctttgtgccagccaggtgagatgccaaccctctagccccatcatggagtccccctttgctttggtggcagacgcagaccccatatgttaactgtaaactcaaatctgaaacgacccatttcccagccctgcttcactgtcagaatgttctggttccctctctaccaggtaaaactctgtctaccctgaactagggatcccagcttctccatcttcctcgcctgattatgaaggatccaagactttcatctttgaatcccctaccctaaagcctggcctgatcattgtgtggttagtgtctgactcatggagttggccagagccctccctcatttcctgatgttttccaggacagaaactggtgagtgactgcacagagttcactgaaacggaatgccttccttgcggtgaaagcgaattcctagacacctggaacagagagacacactgccaccagcacaaatactgcgaccccagtgcgtgcgctgttgggaaagggacgcttgggaaccgggctgatattcccgacaatgcagccattctaattttatgtagccagggtctgctctgattggttggagtccgggctgtactgatcattaaatgatttgattgccatctctacttggaagagggtctgaggaagaaagagcaggcaatgtggggagtgaggctcagagcatggcccagcagggggttcccatccttcctgcccttctcttctcagacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattggtaagtggctcatctgggaatcagttttggagggggacagaggagcttagggcccaaggtgaggggctgggcagtgggcacttagccccagaggcagaggaagcagaggctccaacctatgtcggtatccccactggagtgagctgcagacgggaccttgttcattctgccttctgccatggggatctgcctttgaagggcaatgggagaagtcctcctggggactgcagctgtcgggggcagtaccacatcgggggaagagtgctcaaggcaggagctcttcccgtcctgcctggccactggctgccttgtgagccggacaggtggtccactgtgatggttaatgtccccctccccacccactcccagctacaggggtttctgataccatctgcgagccctgcccagtcggcttcttctccaatgtgtcatctgctttcgaaaaatgtcacccttggacaaggtataagcactcatcccttgtgtttcctgctctaagagtggcatggagctgcctccattctctccagccacctgtcctgtccctgctcccagaggtccacacacactcatgtacttgtgaagcatctgcagagtggcctcatggccaaccagacaggcacatttccacattttttttgcctgctgtctctttgaggtaatagacactgttgatctctcgcttcatgagagcctcctatcttgggggtattgggacacttattttagctttccttctgcccctcctgcttctcctcagttttcctcgtcttgctttcaccttacctggctttctagggctttctgggctctgggtgctcaccctgagggcctccctctcttacctccaactccaaacccacaccaggtcctgccactggctgtctacgtgttttgggaacttactgtctccactgttgtcactttagtttgggcctcatcactgtggtctgggtgatgccttttctgcctcctggcctccctgcctctgtctctcccctcctgctggttctgtctccatcctcttgccaacatgagcgttcgacagtttctttcaaatcatgacactctcctatttgagatgcttcctgtctctctgttggaactaagactccttagcatggcacccaaccttcctgttgcatttcctgctctctttcctgcatcgcatagcttcatgctacttgcaatcctctgaacacactgttcattctcttccatcaaactcatctgcctggaataccttaaacatgggccccaggccaggcgcggtggctcttgcctgtaatctcagcactttggatgccaaggcgggtggatcacttgaggtcaggagttcaagaccagccagcacaacatggtaaaaacccatctctactaaaaataccaaaaaattagctgggtgtggtggtgggcgcctgtaatcccagctcctcgggaggctgaggcaggagaatcacttgaacccggaaggtggagtttgcagtgagccaagatagcgccactgcactccagcctgggcaacagagcgacattctgtctcaaaaaacaaacacctgccccattaactttttgcatttgatttttaaaaatgggcaagataggcacatgggacagaaggcacaaaagagccaaagtgatgtctttctcccatccctgccccttaggctcccagttctttctggagggagccattgttccttgcatatccttccagagattctacatataaacaaaccaacacacacacacacacacacaaacacacacaaaatttccctccttttacttttgcacaaataggagtatacattttatttgttaactgtctgcctttccctaatagattgaaaattccttaaatgtagaaacttggccttttttttttcttccattgatacatcccctatacctggaacagtacctgacgcatggtaggtgcttaaatttttactgataaatgttgactgataactggaggcaccactggtatagtttttttttttttttttttttttttttttttttgagacagagtctcactctgtcgcccaggctggagtgcagtggcgcaatctcggctcactgcaagctctgcctcccaggttcacgccattctcctgcctcagcctcctgagtagctgggactataggcgcccgccaccacacccggctaatttttttgtatttttagtagagacggcgtttcaccgtgttagccaggatggtcttgatctcctgacctcgtgatccgtctgccttggcctcccaaagtgctgggattacaggcgtgagccaccgtgcccggccaccagtggtatagtattaatggaatcagtgcattggcttacgtatctgattacagctcagtaagtgtgtgaccctcactgagcctcagtctcctcatctgaaaaatgggaatgaccttcatttcacaaggcttgagctaaaaacatgtaaagtgtattgtaaattcctgaatgctctactcatgtaagactaaagtaggccgggcgtggtggctcacacctgtaattgcagcactttgggaggccgaggagggcagatcatgaggtcaagagatcgagaccatcctggctaatatggtaaaaccctgtctctactaaaaatacaaaaattagctgggcgtggtggcgcacatctgtagtcccagctactcaggaggcggaggcaggagaattgcttgaacctgggaggtggaggttgcagtgagctgagatcgcgccactgcattccagccagtctggcgaaagagcaagactctgtctcaaaaaaaaaaaaaaaaaaaaaaaaagactaaagtacatggtttcttcaaagcttctctctctttctcccaccttagatgatttttcctttgcaatgtcctgtgtccattccgccccactcctcctggggccacctggaccaggtcttcatcatctcatatctatatgtttgctgtgtctcctggctggccactcttctgtaatttctcctcctctgagctctctgggcagctgaatcttctcactagtgaagtcgcctggttggatgctgatgagactgaccagctgaatccagttgaaaacttcacacttggcagtgatctggttctaaagacacaattttccatagtttcctaacaccatcctgcatgccacctgccttatttccccacatcacatcgtcccacttagcgggactgcactgctgatccaaattttacatcctttagggcccactcaggtcatatgtcctcagggaagtctttctggaagaaccttaaaccagaggttctcaacagggggcagttttgctccctgtggaacgtttgccaatgtctggacacatttcattcgtcacaaacggagagggggatgctacagggatctggcggatagaggccagggatgctgctgaacatctgcaatgcataggacagcccacccccacccccacacccccagtaaataatgatccagcccaagtgtcactggtgctgacgttgagtaaccctatcttaagctgaactcatcatctctccattccagccttggtggattctgtctcctctgaaccattcccatctcactttagcctacctagatcacaaagcttggcactcattatagactcccctatttattactccttcaagatgtgcaagaatcttttctctgcacttttaagttctgtaagaagagtctgtgtcgttcctataataaccagcataggacgttgcacgtgttgtgtgctcagtgaacctggatttgttgattgttgactgactcactctagagttggaaatcttatgcttggggaaacttaatatctctttctttctctgtgtgtgtgcatttgtgcacgtgtctgtgcatagctgtgagaccaaagacctggttgtgcaacaggcaggcacaaacaagactgatgttgtctgtggtgagtcctggacaatgggccctggagaaagcctaggaaggtgggaactgaagggggagatgaggcacacaggaacactggatgggaaaaaggggaggggaggcagtttgggggtgtggtatcacagctctgccacttatcttgggagtctgggcaaatcacttcccctctcttagcctcagtttcttcatctgtaaaatgggatgataacagcacttccttagtaggttttgattttagagtgagaaggttggcctacagtaaagatcagataatgtaaatcagtgaaaaaggtcaggggtaagaaaattacattctctttacctaacgctaaatgaccagttaatgggtgcagcacaccaacatggtacatgtatacatatgtaacaaacctgcacattatgcacatgtaccctaaagcttaaagtataataataataaaatttaaaaaaacgaaaaatacattctctttgctttttctcaaaatgtactttcctctttgtagggctgggactagaatgaggtgagcaaggcacttgccctcgggcgcaatatttaagaaggtgccataaaagtgtagtaatcaaggtaaattcattttgatgcaatatttttaaaaataaaaattaatgcaaagaaatccatgatgagcaagatagcaacattttaaataaagaacaggatccgaccctgtgtttgcatgaccctgcctcactcacctcaccctaatcctggccctggttccagtaaaaggaataggcagccagcctgcaggccgtagtttgctgacttggtgtccgcctgatgattttcaaaatatggcattaaaagaatgtttaccttgatgactgagtgttttggacatccttttcaattttgtcctgaaacaatttcatcccttgcctcacgctagtctccgccctgccttttggtctttcttttattttcccactttgaaaaaaaaattcggcatgagaaatactttacctttcccctccactcttctataccaaaagcaacatgcagacatgaatcatgctagacctcggcattgggcagagagcagggagtggcggggagcatggtgagcaggtggtgacagccactgccaccactcgcttctagatggttcccaggtggggaggctgccaactggaacccagtcttcccagtttgtaagagaaatcagatgtctaggtttgaatatgtgatctcccagtttaaaaatgtcggcaaatatttccaaacgttaagaaaatgttctggctcctttaaagacatctgccagccacatttccccaaggaccgcggtttgaaccttctgatgtagatgagctctgacattggaagattctggagtctgacaagtcacagcaggttgagggtagggagaaactgcaggtgaggggtgcatgctgaagtcctgatttctccaggtccccaggatcggctgagagccctggtggtgatccccatcatcttcgggatcctgtttgccatcctcttggtgctggtctttatcagtgagtcctcaggtggggaggtgttgggggagggaggggagaccacctgtttcttatctggcctctccaactccccatcctttttttttttttttttttttttagaaaaggtggccaagaagccaaccaataaggtaggtcacccctgagaacccgggacagagttttgacaaactgggaagatggcctcacggttgcctatggggcagtaaaactgattcagagtctgtctctgcagccagtggggtggcagcagaattggggactgtcatccccacccaccatgctccttccatccagagctcaatcccccacagaactgcccctggcaccactggcagagcctaacactggctgttcttcactcctttcctggcattcaacgcgtggggagctgcatctttgggccttggggctgggtcaaatgggtgggagcaaatgtggcagccccttaagcccactggctcccactctggaagctcttcgtcgcccttggtgtggccagcagggggcaggaggcacccgaggaatcagcactgacccgccgtctgggaaaggggggagggcttggggaagggatccgcttcccagggaggggctcctcagaggcacagctgcccctgctgctgggggtgacctcacaccttgcctctccaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctggggg

SEQ ID NO: 7 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEMLPPAARRQDPQEMEDYPGHNTAAPVQETLHGCQPVTQEDGKESRISVQERQVTDSIALRPLV

SEQ ID NO: 8 - DNA, 16047 bp, Mus musculus
  source 1..16047: mol_type = other DNA
ccccgccctcttcctgggcgggactcctagcagggactttggagtgacttgtggcttcagcaggagccctgtgatttggctcttctgatctcgccctgcgatggtgtctttgcctcggctgtgcgcgctatggggctgcttgttgacagcggtgagtggcttgtgttctaacctccaagggagttagggcttagagagtgagagatggaaagaggaaagaggagacaagactttggagatgagagatcttcctactggaagcggcggttagtaggatgggcaagatctctcgcgtcttgacacacacacacacacacacaaatgaggtgggctgctcctctttccttccagaaggtcggggttctgttccacgaagcccacaggggaaccttagggagggcattcctccacagcggtgcctggacagctttgtctgacccaagccttggctccggagctgactgcagagactggaaagggttagcagacaggaaagcctggctagggggaagggcgggtctggcctgtttcctgtcactttcccattgtggacagatgtctgccacctgtggttatcttctccttgccagttggggccactttgtctagggaatcttgtgcgaacaagaccccaggtactttttagggaagaggtaatttactaaccaccatacccgtatcatagctgagccaatctagagaaatccccaagtttgtgcctctgcctcatgagcttaaggtggtacaggagacacccagaggatgaggaggaagaagaggaggagggggaggaagaggaggagggatagctttagaacccaagaaaggataagagaccagactaggtttctccaggcaccagcagggctcagacccaatcaagcgcatcttggggatttctagaagccggcacttgattcgcttttggtttaaatgtatttctgcagttcctcattctggaacctgggaatcccctgactacctgagtctcagcccctctcctctggcgaggctcccctagctttcaggcagggtagattctttccggttggtgacagggacttcggtaacttcatgggttctgaattgtccacccaggaaggtgtcgtggttcaggactggctttctgcagctgggacagtcagtgccctaagcacatccctgtccatcagccaatgtcacctgtccatcagccaatgtcacctatcccaggttggtctagttgagaatgacctttcacattcttctttttcttctgttcacgcatggtttgtgtatatgcgtacatctgtgtgggaacacacgtgtgcttgcatgcggtagcctgaagttcatctcatgagtcatccttggacacggtcccttcttagtcatccacacagggtctcaagcaaacccagagctccccatagggcgagtctcacacgtcagctggctctggggaccctctgcttctgccctctgaagctggagttacagggaggccactacaaagacctggtgtttgcacgtgtttctggagatcagaattctggcccacccatttatatggcaaacaccttgagagctcagtcatctctcttgtccttgaatctctgatcctcctgccttcacctccctagtgcatacaccaccatgcccagttttatgtggcacttagaatagaatctagggcttcatgcatgctaggcaagacactatacttactgagacacacccccagccaaaggacatagactctgacatgtggcctgccaagaatcaccatggaggttagccgcaggaaaggagcagaagcgagacagttgctttactagttttcattttttaccccaggccagtggttcctttcgcagaggtggtctaagaccatcagaaaacacagatatttacattaggattcagaacagtaacaaaattacaattatgaggtagcaacaaaaataatgttttggttggggggggggtcagcaccacatgaggaactgtaatgggccccagcgttaggacagttgagaaccactgctctaaataagacttttagggagactggatgcaggaagacggtgaggtgagagagaagtggatgagggcctttgggagggatggaggaagaaacagaggtgtgatgaaggctagtctggaccactcaagaacaaagtctgcccttgtaagccccaatctgggctccagggaggggctgggccctgagagaaatagcaggtgttctttactgacctcttcctgtgcagggggctgtgagggagactgcatgtcaccttcctgtgtgagggagactgcatgtcaccttcatttgatgtctcccaacgtctctgttacacggatgaccaagatgaggctcagaaaggggaggggttttacccaaagcctcttgacatggatatgaagctatgtctgcctgacttctgagcctgccactcacgcggcaaggggtcttcaagctgtggccctgagacctattgtggaggaaggaacaatgctctgggggctggaaactggggctgactggccatcagagggaaagtgtcttttccaaaggtgccattgaggatgtctactgttctagaaggaaggaaggaaggaaggaaggaaggaaggaaggaaggaaggaaggaaaggagggagggagggagggaggtgggaagtctcagggaagataaaactcagggatgatgccctgctgggggcaggtgctttcaggtacatttcattaagtgatttgctcagatacccagtttgagtgagagttctcaatttcctggattctggacagtcagacagctactagggtgcaggacaccagggcagacccccatcatctatgtgtgctgattaaagctgccattttctgaatactaccttgctatactgccaggccatgggcaagtgttacaatctcattttaaaggtgagaacacagggctagagagatggcttagtgggtaagagcgcacgctggctgccatagccacattgggctggtacataaattcaacaagaataataaaaacaaaggaaacacagtcttagagaagcaaaataattaggatggtgtactggctagttttgtgtgtcaacttgacacaggctggagttatcacaaagaaaggggcttcagttggggaaatgcctccatgagatccagctgtaaggcattttctcaaatagtgatcaagtggggaggtccccttgtgggtggtgccatctctgggctggtagtcttggttctataagagagcaggctgagcaagccaggggaggcaagccagtaaagaacatccctccatggcctctgcatcagctcctgattcctgacctgcttgaattccagtcctggcttccttagtgatgaacagcagcatgaaagtgtaagctgaataaactctttcctccccaagtgcttcttgatcgtgatgtttgtgcaggaatagaagccctgactacacagatgggatacagagtggtttgtcccgttctcaaatgtgggctccacgcaatcggtagctgtggttgtttctaaaatgtaacctggcttcaaggagcacacgagcaagccggagggcttagctctgcagttaagagcatgtgctgcccttgcagagagctggaatccgattcccagctcccacattccacagcccccagatatctgatcccctcttccgagggtacttacacccacacatgttcatattcatatatctacctataatcaaacatgaaataaatcttgtggctagagagatgctcagaatattgttgctctgggagaggacccaggttcagttttctagcacccacgtgtggcccacaatcatcctcaacaccagttccagggaacccagtgccctcttctgacctccaagggcaccaggcacgcatgtggagcatattcaggctacacttgtacacataaaataaataaatctaattatttttaaagtcttaaaaaacaaacaaacaaacccccaaacaccgtcgcctaaaacctcaaggatagggttgtgactgaaggcacttgtcatataagcctgaggccctgggttcatttcctggaacccatgtaaggtagtgtgtggtggcagaagtccaagtctgacatcctaaggctcctactgtgagacaggaggtgggaacaggagaaccccggaaactttcagcacatgcaaccaagaacaacaaaatgatggtggcttaagcacagtaggagatagcactgagagcagtgttggccactgacctccgtacacttccagttatggcaagagcacgctccaagctgcagacagggcagtgggcgttgcgtgtgcacacacaccaaaacacgggcccactttatggttcttaaaaaaagtttctacacttaaaaaaatgtagttttcgttatacttatttatttatatattatttgtatgcatgcatgcatgcatgcgtgcttgagtaccaaagcgtgtgtgtggtcagaggacaagttagggaagctggctcttcctaccatgtgattctaaggaactgaactcaggctgtcaggcttggcagcaagagccttcaccagctgagccatctctccggcacctctctctctctctctctctctctctctctctctctctctctctctctctctcctgtttgtatttttcttttttaaaaatattttttcaagttttttttttttttaaagatgtatttattttatgtgagtacactgtcactgtcttcagacacaccagaagagggcatcagattccattacagatggttgtgagctaccctgtgcttgctgggatttgaactcaggacctttggtcagtgttcttaactgctgacccatctctccagccctgtttgtatttttcaacacaaggtttctctgtgtagccctggccgttctagaccaggttggcctcgaactcagagatccacctgcctctgccgcctgagcgctgggattaaaggtgtgcgccaccactgctcagctgactgtttctggaagtgttacagctccgctctgagggaaacttttctctgagtgttgtagatgtgaggagaaatcccgaaaggcttccctgtggaggtgttgcatctctgaaaggaaaagcggagtatcacaaagtcccactgctggacaaacctcagagagtggctgcctccgcccaggggcgaagcagcagagagctgagctgcaggcagcttaggcagttctccagggtggagtccttctgggcagggattggtgagacttcatgctcaaggattggtgggttcgcatagttcttttattttttcccaactaggaagtgggctcaggcctttcccccagctagagtttcactgttctttaaaaaacatcattttgtttttatttcatgtgaattggtgttttgccctgcatgtttctctgtgtgagggtgtcagattccctggatctggagttacaactggtggttgtgagttgccatgtggatgctgggaactgaacttgggtcctctgaaagagtaaccagtgctcttagccattgagccatctctccagctcctccaaatccttttcttattcatttaaaaaattacatgtatgtatgtatgtatgtatgtatgtatgcatgtttgtacgcacacacacacacacacacacacacacggagattagatgctaacttttgagagttggttctctccttccatttctgggccttgaaattctggttatcagtcttggcagcaagcgccttgattggctgagccatctcgctccttggttcttcaaggagtaagtctctggcgctagctagatcatagttaatgccttttttttttcttttttctttttttgagacagggtttctctgtgtagctttggctgtcttggaactcactctgtagaccaggctggactcacagagatctgcctgcctctgcctctggagtgctgggattagaggcttatgccaccacagttggccagttaacacctctgaaagacttgctaccaacccaccccaggcttaaaagtaaaatcaagagcagacagagcgaaggatctcagcaaagaaagctacgcatcgaggcttaataaccctgttatgaatctgttgagtgtatttttagggtttcttttaatttataggaagtgatacttgctgacctcttgatgcagcagtagaagatttacagttaaaagaagtgtgcttaaattagcaagaagcagctcatagcatgggtggtccccggatgttgtagaaacacatgttgagagtcccgcccctgtggactctgttcagtgttgccctctgtggggtgattcttatctctttggtggcagggagctggggacagaaaccgggagaagggctgaggccagcttgagccagcagtctcgggactctggaggaagaactggagttctccctacctgctgcgtctttgggagcactgaagagtcctgtgcatctgttcggattagagggttctgcgttcttgctttggtagatggcagtaagacgatgtgacaacagagtaaaaaaaaaaatagacctcacactctgggggctcacttttctgctttggatttccacatcagctacagcctgcgtcttggctaactttcaacatgccggtggaagatcccttccagctgtccacttctgtttttaggtccatctagggcagtgtgttacgtgcagtgacaaacagtacctccacgatggccagtgctgtgatttgtgccagccaggtgagatgctagccctcctgccccgtaccaagaccctttccctcttggattgctggtggatgcagaccccatatatagactgtgaactcaagtctaaaatgacccatttctccctcttccttgatgccagaataccccaagctgtcccgtctcttccatcttccttactcgtgtagggtctgagatatccattcccaaactccaaccctctcacctccagtcctggctccctgggttgtgacacagtctgtgtcacaggattggcccaacctgcctcatatcctcctgtttttcaggaagccgactgacaagccactgcacagctcttgagaagacccaatgccacccatgtgactcaggcgaattctcagcccagtggaacagggagattcgctgtcaccagcacagacactgtgaacccagtgcgtggggctgcctgggaaggggtacttgagaaccgggttgatgttcctaatgctgaaatccctctgttgtcagtggccagggtctttcctgtgagctagagtctgggttgaaggggctagttgactgacatctgtactgggaagagcgagaaaaccagcagatgacgtgaggagtggggtcctggctgcggcccagcgggttttcccattcttccttctcatctccgctcagatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatgggtgagtggcctgcctggggaaacagctctgtgggtgggagagctggggtgagctttgggtctctggcctccagaagctgagggcagagaaagtcccacctgggctgggatctttcatttggatttggacctgggctctgggcagcttcctgcggggttgtggccttcaggggctgtgttgccttggggtaagaagctgagggcaggtgttctgtcctgccctgcttgtctgctggctcccttgggagtcagacactgtggcccaggtgtctgctcatgcatctttccgcatccttccagccactgagaccactgataccgtctgtcatccctgcccagtcggcttcttctccaatcagtcatcacttttcgaaaagtgttatccctggacaaggtataagggtcacctctccctaaccaatgacagggtgggtcttgtctcagtctctttagccacctgctgttcagtccctgactttccccacccccatggtgggtcacttactggtgaatgtgaccttgtggctggcttaagggacactttgtgcagttcttttagcttgcttctgcttagttaatagaagcctgttggtctccatatcctcttgaagtctcttcttaaagcatcatgacactcgtactccccttttcagttcactttcttggtgggtatgtgtatgtgtgcatgtgtatgcatgtgcatgcatgcatgtgtgtgtgcatgcatgtatgagcatatatatgcatgtgcatgcatggatgcatgtgcacacatgcatgtatgtgcatgtatgtgcatgtgcattcatgcatgtgtgcatgcatgtgtatgcatgcatgtgcattcatgcatgtgcattcatgcatgtgtgtgcatgtgtcagctatcagtttggtgtgttcctcctcaggtactgtccactgttttttgtttttgtttgtttgtttgttttgttttgttttgttttgagagtcccaacactgggactataaccacttgctaccaagcatggattttttttttttttttatcgtgggttccagggcttgaactcgggcccttgggttcacagtgctttcctgagtgagttatcaatgcccccagcccttctggactcttacttgtgtttgttgggctttgtctgggttctctgaccacttctttccacctctccttggctgtcttatctacatctatggctccatctccccctggatctgtccttcagcccccagatggtcaagtcccactgctgagtgttatggatcaccagcacctcagactctgtgcatccaaaatgggcccctagccaatccctgagccccagctgacaggcaggttctggctccaggcagtcacacagtgagctgcccacccttgccttcaactccctgtcagtcaggcccatatgactgtcttgggaatccacctcctcactctctagagtggtcacgtgagttcgcacctcctcactgtggtgtggtgatggctctgtctcctcataggctccctgcctctggtgtctcccttggcagctttgcaccatgctgctgttgggagcttccgtagccccttttggatcatgtcgcttcatgatttaaagcactccacgtctccctgctgcctttgggaggaagcccagactcccgggactggggctgggccttccagctacagttcctgtctctccccccacacctcgcaccctgcacttgcaccccgggcacactgtttattcttttgcttcaaagtctcctgtttgggagaaaacaatatatatgccctattctgttttttatagttcacatttaatgttttttaaatgggcaaacctgtcacctcaggctgtttcctcactactccttcagagagagagagacagagagagacagagacagagagagacagagacagagagagacagagagagacagagacagagactcagagacagtcagagagacaaagatagagacagagaaagagacaatcagtgagagacagagggatagagagagacagagagtcagagagacagagacagatagagagacagaaagacagagacagagaggtggggggaggagatagggggagacagagggacagagagacagaaggacagagaaactgctaataaacaaacacattgacataccactcccctctgccatgatacacatatgcacacacacacatacacacacacacacacacacacacacacacacacacacacacacacacacacacacaccacagtggtttctcttgtggttatagtgcttgcttgcaggtcacactcaccgtcctcactggtgggtttagttgttcatagaagttcccgtcccggagtcaaatgtgcaactgccgcactgccccacttagctggttcatgctgctgtttcaacttttatcccctttggagacccttcggatcttctctggggacccccaaatctgcctcagtttgtgtgagaccctcagggatgcccctaatctcgggaggcttcagccaacttgtaaaggtgctgagggcctttctcacacaaggctagagcacgcacgcattttctctgaagcctctcttccaccacatcccggttttccttcgccatgtcccttgccatgtcccctctgtcccctcctcccaggaccttccatccacaccacacatctccactcctgtcctgcctctggctgcccactctgctgcaacggtcctctcaccagtcaggtcactccctagctgcctgcctgcagctgccctgcctgcagctgctcatcaccgcacttgtcaccatgtgactcccctcctgtcacactgttcctacttagctgggtcccactttcccatccttcaatgccttctgatgccccccttcatggggaagtcttcccagaagaccctgaaagcagagcttcttaacggggaccagtttggctctctgtgggggcgttggccacatctgatcatcttttggatgctgtacttggcggggaggctgtttctggcatttggttagtggaggcaaaggtgctgctaaatagtctgtgaaacaaaggccatttcccagcacaaaatacctgcagattggccatttccatttcaatatgaccgtaacctttcctttctgtggtttctgttcttcacttaatgatcatcttgggatgctgcactctaagtcacgtgctcagtgaacaaggacttgctgcttgctgggggactcccctgggcttggaagtcttatggcggggagccctgtttctgtctgtctgtctgcatgtgtgtgtgtacatgcacatacatgtgtacacatgtgtctttgtgcagctgtgaggataagaacttggaggtcctacagaaaggaacgagtcagactaatgtcatctgtggtgagtccaggggagaatggccttgccaagtctttgggaagcagggaactggggagagactgaggcacgcaggaacactgactgggataggagtgagaccaagaggcagtttggggtacagtaccttagctcccgtcttgggagctgggtaagtcacatcccttgtctgagcctcagtttcttcaattgtgaaataggcccacagcagctccttcctcccttacctgggtcgtgtaagtggcattggaattttgcagtttggaagctgctgccccttgcttgaggttcaggttcactgtgacagtgtcacctggtaaccccagtttggatgctaggatgtaaaacttgaccatcccctaatggatcacaatctcagataacaatagagaccaggccacttttgaatgagtgaagacagagaagggtaagagagctaggtctgatgagcgggcctgtcagcgcagctaattagaggcaagagctttgtaagttcaaggctagcctgggcagcttagaaagatactggttcaacatagaaaagggctgctgagatgggtcagtaagttaagctcttgcctgatggcccagcttccatccccagcatccatggaaaggtagaaggagaggatcagctcctaaaagttgtcctctgaccgctgcatgtacagcacaacacagcacagcacatgtgagtgtgcatacatcatgcacacacatcatacatacatcatgtgcacatatcatgcacacacatcatgtgcacacatcatacacacatcatgcacacacatcatgtgcacacatcatgcacacacatcatacatacatcatgtgcacatattatgcacacacatcatgtgcacacatcatgcatacacatcatacacacacatcatgcacacacatcatgtgcacatatcatacacacatcacgtacacacatcatgcacacacatcatacacacatatcatgtgcacccatcatgtgcacacatcatacacacatcatgtacatgcacacccacaatagtgataaataaaagtttaaatatgtttctagggctggggaggtggcacttgctgttctgacagaggacctgggttcagtttcctgagcccatgtcattgcagtataaaactgtccatgactccagctcctggtgatctgatatctctgctggcgctaggcacatacatgatgcacgtacatacctctagcactttctgatatacataaataaaaatagatacaaattaaaagacattaaaaaaaaaagtaagaagatagctgggggtggggctcagtggtggagcagttgtccagcatgctgaggtcctgggttagacccccacacttgcctagcatatgtgaggtcctgggttagacccccacacttgcctagcatatgtaaggtcctgggttagacccccacacttgtccaggatatatgaggtcctgggttagacccccacacttgcctagtatatgtgaggtcctgggttagacccccacacttgtccaggatatgtgaggtcctgggttagaccagtgccacaaagtaaaaaagaaaataaaagtgcagtctctttgctttttctcaagattgcctttttgtcttggaaaggcgtgagtggatggagtgtgaaaagcacttgagttcatgtgtaacatttaagaagacatcgagaagggacagtaagcaagagaaaacattttgagatgatgctagaaaaaccaaaaacacttttaataaatctgaaaccaaaaaacatgttgatgcacaacatagcaaaattttaaattaagtaagagctgggctggcgagatggctcagcgggtaagagcactgactgctcttccagaggtcctgagttcaaatcccagcaaccacatggtggctcacaaccacccataatgagatctgatgccctcttctggtgtgtctgaagtcagctacagtgtacttacatataataataaataaatctttaaaaagtaagagctgaccttgccagactcgagaccctgcctaccttcttcatcctaaccttggccctggttccaatacaagtttatctaaaagccaggtagccacctatggacctgtgtttgtttcactggtgtctgcctcccgacccatgagctgggtgtggtggtacaggcatgagagccccaagtttaaggccagcctgaaccacttagtgagaccctgtctcaaagaagacttaccttcatgacagtgtgtgtgtgtgtgtgtgtgtgtgtgtgtatacacaccccttgcctctaagagtacctgccttttggtcttttcatttgtcacttagaatagcagtgcagacacagagatactttgtttctctccgccatacttccatagcagatgtaacaatttgggggctagtgtagggaggggtaaagcaggcaggcaggctgtcccttcaagacacagctgacgaacacaccagatgggaagttggctcactgggggcttctcattccaactgcaatctcccagagctagagactgctcaaagggtaggagtatttactatgcaagcatgagctaggtgtggccacaccacatgtgactgtgaccttaataccatgtgtaggggggtggcactagaaggagaattgtagggtgttgcctcccagactagctccaggctcaagagactctgtctcagcagaaggtgcaatatggtggatcaggacaccagcaccctcctttggcctctgcatgtgtgcacagatgcacacatctaaacacataccaggcccacatacaccgtaccccaccaccacacaaagatggactctctcaatttataaacactggcaaataccataaagcgttttaaaagttcccctagtgaccacattacccacaatgctactgcttaaaagctctgacctggattctgggagtgtacagatgctttgggcaaggagtgggagcacctgccagtgagagcacctgccagtgggagcacactccattgggagcacccgccagtgggagcatctgccagtggggtccactcaagtctgtttctccaggtttaaagtcccggatgcgagccctgctggtcattcctgtcgtgatgggcatcctcatcaccattttcggggtgtttctctatatcagtgagtgctcaggagaggaaagggagggagggttcagccctgtcgaaccagcctcctgactcaccctcgcaatgtcccacaccccttcttcttctcactagaaaaggtggtcaagaaaccaaaggataatgaggtaagccatccctgagggagagatgctggaaagagtgactggtgggcagggagggaggctcacggcgtagggagacagactcagtaagcagagagcttgtattggatccttgagtgtggacccatggaaaaggcccattacacccacgctggtgggggcggggagagggggggaggatggacacagggatcttaggagcttgctagccaaccatgggctactccaggttccaagagaaaccctgactcggaaaataagggttaagagtgcaagaagacacaagatgttgacctctagcctctaataatgtgtacatgggtgtgtggaccctctacgccatgagcatacacccaataccacgccacactccgcgcgcgcacatgcgcgcacacacatgcccaaacaggtttagggtccgttccctggaacatataggtgggctactcgcacccccacccagccctgctctcagtctccatcgcttcctcctactcaactacttccccttagggcagagctgggcaccactggcagagaaactctggctgtgctttcctccagccttgaatgctggggatgggagtcggcggcggggggtgggggtggggggtgggggtgggtggatcccgccttcaggggccagtaggtggaaccaaaggggcagtttctcctgctggtctgcagtggctctggaaatttcctgccaaatttcatgtgtccagcagggggcagaaggcatccaagaaatcagttttggtacacccccatcctcccaccccattggaaaggacttgaaggagggattctattcctcagaggcagggtggctctgtggctagaggtgacattggaccttataccttgactccccagatcttaccccctgcggctcgacggcaagatccccaggagatggaagattatcccggtcataacaccgctgctccagtgcaggagacgctgcacgggtgtcagcctgtcacacaggaggatggtaaagagagtcgcatctcagtgcaggagcggcaggtgacagacagcatagccttgaggcccctggtctgaaccctggaactgctttggaggcgatggctcggctcgggagcaggggcctggctctgaggactgcttgctgacctttgaagtttgagatgagccaagacagagcccagtgcagctaactctcatgcctgccccctatcatttctcaacttgctttttaaggatggagggagagctcgggcatcgggggtccacagtgatacctaccaagtgcagcagtgcaggacccagagtcgtcttgctgcggcgttcactgtaaggagtcatggacacaggagtccgtggcccacagcttgtgctgctagagggcacctggttgcccatcagcagggtactggctaaataaatctgtaattatttatacaatgacatctcagaaactctagcaggtggggcagaaaacaggtagtagaatgatgggtagagaaatagcttttaaaacacattccaaggcaggtaagatggcttttgtgagtaaaggagcttgctgcccaaacccggttacctgattttgatccctgggacttcatggtaaaagggagagaaccaaatccagagggttgtcatttgacctccatgtgtgctctgtggtaatgtaccccgtgtgtgcacatgtgcacatatcctaaaatggatgtggtggtgtattgtagaaattatttaatcccgccctggggtttctacctgtgtgttaccatttagttcttgaataaaagacacactcaacctttatatttacaataa

SEQ ID NO: 9 - RNA, 23 nt, synthetic construct
  source 1..23: mol_type = other RNA; note = single guide RNA (sgRNA) for Exon 1 of AIRE
gcaccgcaccgagatcgcggtgg

SEQ ID NO: 10 - RNA, 23 nt, synthetic construct
  source 1..23: mol_type = other RNA; note = single guide RNA (sgRNA) for Exon 3 of AIRE
acctaaaccagtcccggaaaggg

SEQ ID NO: 11 - protein, 15 aa, synthetic construct
  REGION 1..15: note = Peptide tag
GLNDIFEAQKIEWHE

SEQ ID NO: 12 - protein, 6 aa, synthetic construct
  REGION 1..6: note = Peptide tag
HHHHHH

SEQ ID NO: 13 - protein, 198 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLPRQLRRILLPLYEVDDLRDAFRTWGR